(19) United States
(12) Patent Application Publication
(10) Pub. No.: US 2025/0000937 A1
(43) Pub. Date: Jan. 2, 2025

(54) METHODS OF USING CYCLOSPORINE A OPHTHALMIC GEL IN TREATING MODERATE-TO-SEVERE DRY EYE DISEASE

(71) Applicant: ZHAOKE (HONG KONG) OPHTHALMOLOGY PHARMACEUTICAL LTD., Hong Kong (CN)

(72) Inventors: Xiaoyi LI, Guangzhou (CN); Xiangrong DAI, Hefei (CN); Gang LI, Hefei (CN); Guohui ZHANG, Guangzhou (CN); Yixuan JIN, Guangzhou (CN)

(21) Appl. No.: 18/704,457

(22) PCT Filed: Oct. 26, 2022
(86) PCT No.: PCT/CN2022/127767

§ 371 Date: Apr. 25, 2024

(30) Foreign Application Priority Data
Oct. 26, 2021 (WO) PCT/CN2021/126462

(51) Int. Cl. A61K 38/13; A61K 9/00; A61K 9/06; A61P 27/02

(52) U.S. Cl. CPC A61K 38/13; A61K 9/0048; A61K 9/06; A61P 27/02

(57) ABSTRACT

Disclosed herein are methods of using cyclosporine A ophthalmic gel in treating moderate to severe dry eye disease.

### CROSS REFERENCE TO RELATED APPLICATION

This application claims the priority to and benefits of International Application No. PCT/CN2021/126462, filed Oct. 26, 2021, which is incorporated herein by reference in its entirety.

### FIELD OF DISCLOSURE

The present disclosure relates to methods of using cyclosporine A ophthalmic gel in treating moderate-to-severe dry eye disease

### BACKGROUND

Dry eye disease (“DED”), as the 2007 International Dry Eye Workshop (DEWS) defines, is a multifactorial disease of tears and eyeball surface, which can cause eye discomfort, visual disturbance, and tear film instability, and can damage the eyeball surface. Such disease is normally accompanied with increased tear film permeability and ocular surface inflammation. According to the studies on the dry eye disease, in the United States approximately 3.23 millions of women and 1.68 millions of men, i.e., a total of 4.91 millions of Americans over the age of 50, suffer from the dry eye disease. The prevalence of the dry eye disease is as high as over 35% in relatively large epidemiological studies. A survey on the prevalence of dry eye disease in a physical examination center of a Chinese hospital showed that, among the 393 subjects who received the physical examination, 89 patients had dry eye syndrome. The prevalence rate is 22.5%, which is higher in female than that in male. The prevalence of the dry eye disease further increased with age. (See, He et al., J. Trad. Chin. Ophthalmol., 17(6):357-359 (2007).) The dry eye disease can mildly cause foreign body sensation, photophobia, and vision fluctuations in the patient's eyes, and can also severely cause secondary infections, corneal opacity, corneal ulcers, corneal neovascularization, and even corneal perforation and blindness. In short, the prevalence of the dry eye disease is very high around the world, and it seriously affects the vision of patients, eventually causing serious consequences, such as corneal perforation and blindness. It also increases medical expenses and severely reduces the quality of life of the patients with the dry eye disease. Therefore, the treatment of dry eye syndrome is of great clinical value.

At present, there is no effective drug to cure the dry eye disease. The existing drugs are mainly to improve the symptoms and/or signs. According to the severity of dry eye (4 grades), the guidelines of the International Working Group on Dry Eye Disease provide recommendations for treatment. Grade 1: education, environmental/dietary modification, reduction of systemic medication with adverse effects, artificial tear substitutes, eyelid treatment; Grade 2: if the treatment for Grade 1 is insufficient, add anti-inflammatory drugs, lacrimal point plugs, secretagogue, and wet chamber lenses; Grade 3: If the treatment for Grade 2 is insufficient, add serum, contact lenses, permanent lacrimal point blocking; Grade 4: If the treatment for Grade 3 is insufficient, add systemic anti-inflammatory drugs and surgery. Among them, the effects of various artificial tear substitutes, lacrimal point plugs, silicone eye masks, wet chamber lenses, and bandage contact lenses are very limited, because they are not targeted at the various pathogenic factors of the dry eye disease, but only at tears. They are generally able to only improve some symptoms, rather than heal the ocular surface damage and treat the pathogenesis. In patients with the moderate-to-severe dry eye disease, various anti-inflammatory drugs are often required. Corticosteroids, a powerful anti-inflammatory drug, are routinely used by ophthalmologists to control eye inflammation. Short-term application for 2 weeks can significantly improve the symptoms and signs of most patients with the dry eye disease. However, long-term use of corticosteroids has potential side effects, such as corneal epithelial toxicity and increased intraocular pressure. This limits the long-term treatment of the dry eye disease with corticosteroids.

RESTASIS®, a cyclosporine A ophthalmic emulsion, is effective in patients with aqueous tear-deficiency dry eye syndrome. Cyclosporine A can not only prevent T cell activation and the translocation and activation of cytoplasmic transcription factors required for the production of inflammatory factors, but also directly promote the secretion of tears by stimulating nerves. (See, Hiroshi Toshida, Doan H. Nguyen, Roger W. Beuerman, et al. Neurologic evaluation of acute lacrimomimetic effect of cyclosporine in and experimental rabbit drug eye model. Invest Opthalmol Vis Sci, 2009, 50(6):2736-2741, Hiroshi Toshida, Kiyoo Nakayasu, Atsushi Kanai. Effect of cyclosporin A eyedrops on tear secretion in rabbit. Jpn J Opthalmol, 1998, 42:168-173.) RESTASIS® (0.05% cyclosporine A ophthalmic emulsion) is now the only FDA-approved prescription drug for the treatment of the dry eye disease. It is used for the patients with the dry eye disease (tear deficiency dry eye disease) in which tear secretion is suppressed by inflammation of the eye and is associated with chronic inflammation. A recent study showed that increasing the number of administration of RESTASIS® to 3 or 4 times a day can achieve a better treatment effect than 2 times per day. (See, Dastjerdi et al., Cornea. 28(10):1091-1096 (2009).) It was reported that, 6 months of treatment (twice daily) of RESTASIS® could significantly improve corneal fluorescein staining and Schirmer's test results in the treatment group compared with the control group, but 17% of the patients had ocular burning after using RESTASIS®. (See, label of RESTASIS®, https://www.accessdata.fda.gov/drugsatfda_docs/label/2012/050790s020lbl.pdf).

Cyclosporine A is a selective immunomodulator that decreases T-cell activation and T-lymphocyte infiltration of the lacrimal glands, inhibits the apoptosis of ocular surface epithelial cells, and is effective in treating DED. The mechanisms of action of CsA mainly include increasing tear production, reducing the release of inflammatory cytokines, and protecting conjunctival epithelial cells. The Chinese expert consensus on DED (2020) recommends using CsA ophthalmic formulations in the treatment of moderate-to-severe DED with ocular inflammation. The Dry Eye Syndrome Preferred Practice Pattern by the American Academy of Ophthalmology suggests that topical cyclosporine can lead to long-term treatment-free remission of DED symptoms and signs. The CsA ophthalmic emulsion 0.05% (twice-daily, Restasis®; Allergan, Irvine, California, USA) and CsA ophthalmic solution 0.09% (twice-daily, Cequa; Sun Pharmaceutical Industries, Cranbury, NJ) have been approved by the FDA for the treatment of DED. There is only one generic CsA formulation 0.05% (twice-daily, Shenyang Xingqi Pharmaceutical Co. Ltd.) approved for DED treatment in China. The most common side effect of ocular CsA treatment is an ocular burning sensation, and other side effects include blurred vision, ocular itching, conjunctival hyperemia, discharge, foreign body sensation, and stinging. (Peng W., et al. Cyclosporine A (0.05%) Ophthalmic Gel in the Treatment of Dry Eye Disease: A Multicenter, Randomized, Double-Masked, Phase Ill, COSMO Trial. Drug Des. Devel. Ther., 16:3183-3194 (2022)).

Due to the above-mentioned side effects and low bioavailability of the available ophthalmic CsA formulations, there is need in the art to develop methods of treating DED with novel CsA ophthalmic formulation that cause fewer side effects and higher bioavailability.

### SUMMARY

The present disclosure provides novel cyclosporine A ophthalmic formulation in the dosage form of gel, which has higher bioavailability and causes fewer side effects due to increased comfort and reduced frequency of use (only once every night). The present disclosure provides method of treating dry eye disease using cyclosporine A ophthalmic gel. In some embodiments, one dose of the cyclosporine A ophthalmic gel per day can achieve the effect of two doses of RESTASIS®. The present disclosure shows safety and effectiveness advantages over RESTASIS®.

The methods disclosed herein provide improved safety and effectiveness over prior products. The methods disclosed herein solve the eye irritation problem (eye burning sensation), increase the bioavailability of cyclosporin A, and require fewer administration per day.

In an aspect, the present disclosure provides a method of treating moderate to severe dry eye disease in a subject in need thereof, comprising administering to the subject an effective amount of cyclosporine A ophthalmic gel.

In some embodiments, the subject is characterized in a) Eye dryness score (EDS) ≥40 points (visual analogue scale [VAS] of 0-100 points); b) Break-up time (BUT) <10 s; c) Schirmer test result <10 mm/5 min; and d) Corneal fluorescein staining score (ICSS) ≥2 points.

In some embodiments, the subject does not have an alanine aminotransferase (ALT) level ≥2 times the upper limit of normal, an aspartate aminotransferase (AST) level ≥2 times the upper limit of normal, or a serum creatinine (Cr) level ≥1.5 times the upper limit of normal.

In some embodiments, the cyclosporine A ophthalmic gel is administered to the patient once a day.

In some embodiments, the effective amount of the cyclosporine A ophthalmic gel is one drop per day.

In some embodiments, the concentration of cyclosporine A in the cyclosporine A ophthalmic gel is from about 0.05% to about 0.1% by weight.

In some embodiments, the concentration of cyclosporine A in the cyclosporine A ophthalmic gel is about 0.05% by weight.

In some embodiments, the effective amount of cyclosporine A ophthalmic gel administered to the subject per day contains about 0.025 mg cyclosporine A.

In some embodiments, the cyclosporine A ophthalmic gel is administered every night before the subject goes to bed.

In some embodiments, the time window between the administration of the cyclosporine A ophthalmic gel and other drugs is no less than 5 minutes.

In some embodiments, after being treated with the method, at day 84 since the treatment starts, the subject's ICSS drops by at least 1 point from the subject's ICSS before treatment.

In some embodiments, the treatment period is up to about 12 weeks.

In some embodiments, in a treatment period, the onset of efficacy starts no more than 30 days since the first administration of the cyclosporine A ophthalmic gel to the subject.

In some embodiments, in a treatment period, the onset of efficacy starts no more than 14 days since the first administration of the cyclosporine A ophthalmic gel to the subject.

### BRIEF DESCRIPTION OF THE DRAWINGS

FIG. 1 is the visual analogue scale (VAS).

FIG. 2 is a schematic diagram of cornea divided into three parts, wherein staining in the lower ⅓ is counted for determining corneal fluorescein staining score (ICSS).

FIG. 3 shows the Schirmer tear test results at each time point of New Zealand rabbits given a single dose of test preparation (cyclosporine A ophthalmic gel) at 40 ul/animal in the right eye (N=12). “**” indicates that the average amount of tear secretion at each time point after administration of the test preparation was significantly different from that before administration (P<0.01).

FIG. 4 shows the Schirmer tear test results at each time point of New Zealand rabbits given a single dose of reference preparation (cyclosporine ophthalmic emulsion) at 40 ul/animal in the right eye (N=12). “**” indicates that the average amount of tear secretion at each time point after administration of the reference preparation was significantly different from that before administration (P<0.01)

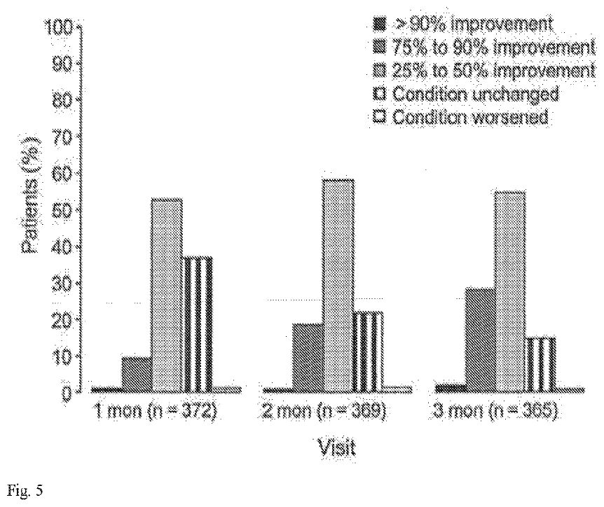

FIG. 5 shows improvement of patients with dry eye disease treated with 0.05% cyclosporine ophthalmic emulsion for 3 months. Note: the data are expressed as percentages: >90% improvement, 75% to 90% improvement, 25% to 50% improvement, condition unchanged or condition worsened.

FIG. 6 shows Schirmer score of patients with dry eye treated with 0.05% cyclosporine A ophthalmic emulsion for 3 months. Note: The data are expressed as mean±standard deviation. *p<0.0001 when compared with baseline (with anesthesia vs. without anesthesia).

FIG. 7 shows the “Outer packaging label sample draft of drug for washout during screening period,” the “Small box label sample draft of drug for washout during screening period,” the “Bottle label sample draft of drug for washout during screening period,” and the “Large package label sample draft of study drug during treatment period.”

FIG. 8 shows the “Packaging label sample draft of study drug for each visit during treatment period.”

FIG. 9 shows the “Small box label sample draft of study drug for each visit during treatment period.”

FIG. 10 presents the participant flowchart of the Phase III clinical trial.

FIG. 11 shows the proportion of subjects with at least a 1-point improvement in ICSS from baseline at day 14, 42, and 84 in the CyclAGel (n=315) and vehicle (n=312) groups. *P<0.025; **P<0.0001. Abbreviations: ICSS, inferior corneal staining score: NR/LOCF, not reached/last observation carried forward.

FIG. 12 shows subgroup analyses for the proportion of subjects with at least a 1-point improvement in ICSS at day 84 from baseline. The squares represent the mean value, and the lines represent the 95% CI. Differences between groups with 95% CI including 0% are considered not statistically significant. Abbreviations: CI, confidence interval, ICSS, inferior corneal staining score.

FIG. 13 shows changes from baseline in DED signs of the study eye at day 14, 42, and 84 in the CyclAGel (n=315) and vehicle (n=312) groups. (A) Inferior corneal staining score (ICSS); (B) Oxford scale scoring of corneal and conjunctival fluorescein staining; (C) break-up time (BUT); (D) Schirmer tear test. *P<0.05. **P<0.01.

FIG. 14 shows the Oxford Grading Scale.

FIG. 15 shows the proportion of patients whose ICSS decreased by ≥2 points from baseline at D14, D42, and D84 in the CyclAGel group and the vehicle group.

FIG. 16 shows the keratoconjunctival staining scores at D14, D42, and D84 in the CyclAGel group and the vehicle group.

### DETAILED DESCRIPTION

### Definitions

As used herein, including the claims, the singular forms of words, such as “a,” “an,” and “the,” include their corresponding plural references unless the context clearly dictates otherwise.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

As used herein, the term “about” means approximately, in the region of, roughly, or around. When the term “about” is used in conjunction with a numerical range, it modifies that range or that numerical value by extending the boundaries above and below the numerical values set forth. In general, the term “about” is used herein to modify a numerical value above and below the stated value by a variance of ±20% or in some instances ±10%, or in some instances ±5%, or in some instances ±1%, or in some instances ±0.1% from the specified value, as such variations are appropriate to perform the disclosed methods.

As used herein, the term “comprising” encompasses “including” as well as “consisting,” e.g., a composition “comprising” X may consist exclusively of X or may include something additional, e.g., X+Y. Throughout the specification and claims of the present disclosure, the terms “comprise,” “include,” “contain” and variations of them mean “including but not limited to”, and they are not intended to (and do not) exclude other moieties, additives, components, integers, or steps.

As used herein, the term “treat,” “treating,” or “treatment”, when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art. In yet another embodiment, “treat,” “treating,” or “treatment” refers to modulating the disease or disorder, either physically, (e.g., stabilization of a discernible symptom), physiologically, (e.g., stabilization of a physical parameter), or both.

As used herein, the terms “administer,” “administering”, or “administration” as used herein refers to providing, giving, dosing and/or prescribing by either a health practitioner or authorized agent and/or putting into, taking or consuming by the patient or person himself or herself.

As used herein, the term “subject” refers to an animal. For example, in some embodiments, the animal is a mammal. In some embodiments, the animal is a warm-blooded animal. In some embodiments, the animals are humans, rodents, simians, felines, canines, equines, bovines, porcines, ovines, caprines, mammalian laboratory animals, mammalian farm animals, mammalian sport animals, or mammalian pets. The animal can be male or female and can be at any suitable age, including infant, juvenile, adolescent, adult, and geriatric. In some examples, a “subject” refers to an animal in need of treatment for a disease or disorder. In some embodiments, the animal to receive the treatment can be a “patient,” designating the fact that the animal has been identified as having a disorder of relevance to the treatment, or being at adequate risk of contracting the disorder. In particular embodiments, the animal is a human, such as a human patient.

As used herein, a subject is “in need of” a treatment if such subject would benefit biologically, medically or in quality of life from such treatment.

As used herein, the terms “effective amount,” “therapeutically effective amount,” or “pharmaceutically effective amount” are used interchangeably herein, and encompass an amount of a compound, formulation, material or composition, sufficient to treat or inhibit a symptom or sign of the medical condition. An effective amount for a particular patient may vary depending on factors, such as the condition being treated, the overall health of the patient, the method route and dose of administration and the severity of side effects. An effective amount can be the maximal dose or dosing protocol that avoids significant side effects or toxic effects. The effect will result in an improvement of a diagnostic measure or parameter by at least 5%, such as by at least 10%, further such as at least 20%, further such as at least 30%, further such as at least 40%, further such as at least 50%, further such as at least 60%, further such as at least 70%, further such as at least 80%, and even further such as at least 90%, wherein 100% is defined as the diagnostic parameter shown by a normal subject.

As used herein, the term “pharmaceutically acceptable” refers to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

As used herein, the term “dose” refers to measured portion of an active agent taken at any one time. There may be specific cases where higher or lower doses are appropriate; such doses do not depart from the context of the disclosure. According to the art, the dose appropriate for each patient is determined by, e.g., the physician according to the method of administration, the weight, the pathology, the body surface, the cardiac output and the response of said patient. Provided herein is a dose of cyclosporine A, i.e., the active agent, administered to the patient in need thereof. In some embodiments, the dose of cyclosporine A is about 0.025 mg administered to the patient once daily (QD).

As used herein, the term “ophthalmic gel” refers to a gel formulation used for ophthalmic applications. Gel is a semi-solid composed of a solid, three-dimensional, cross linked matrix of polymers within a liquid, yielding a jelly-like material unable to flow at steady state.

As used herein, the term “cycle” refers to a specific period of time expressed in days or months. In some embodiments, the cycle is repeated on a regular schedule. The cycle as disclosed herein may be expressed in days. For example, the cycle can be, but is not limited to, 28 days, 30 days, 60 days, and 80 days. Further for example, the “cycle” as referred to in the present disclosure is 84 days long. Such cycle can be repeated several times (e.g., 2 times, 3 times, 4 times, 5 times, etc.), each cycle may be the same or different length and can be repeated for a clinically meaningful result, i.e., the. In further embodiments, the cycle is about 84 days.

### Dry Eye Disease

Dry eye disease, as the 2007 International Dry Eye Workshop (DEWS) defines, is a multifactorial disease of tears and eyeball surface, which can cause eye discomfort, visual disturbance, and tear film instability, and can damage the eyeball surface. Such disease is normally accompanied with increased tear film permeability and ocular surface inflammation.

The cause of disease is not very clear. Some researchers consider that it is the progressive infiltration and occupation of the lacrimal gland tissue by inflammatory cells that cause the glands to gradually lose function, resulting in dry eyes. However, recent studies have found that the functional acinar tissue and nerve distribution involved in tear secretion in patients with sicca syndrome are sufficient to maintain normal tear secretion. The decrease in tear secretion is not due to the inflammatory response destroying too much lacrimal gland tissue and leading to insufficient tear secretion, but is related to neurotransmitter release disorder. Pro-inflammatory factors IL-1β, IL-6 and TNF-α play an important role in the occurrence and development of lacrimal gland inflammation. Some studies have shown that these pro-inflammatory factors can reduce the release of neurotransmitters such as acetylcholine and norepinephrine, thus blocking neuro-regulated tear secretion, resulting in tear secretion deficiency, and promoting the occurrence of aqueous tear-deficiency dry eye syndrome.

Dry eye disease can mildly cause foreign body sensation, photophobia, and vision fluctuations in the patient's eyes, and can also severely cause secondary infections, corneal opacity, corneal ulcers, corneal neovascularization, and even corneal perforation and blindness. The prevalence of dry eye disease is very high at home and abroad, and it seriously affects the vision of patients, and eventually causes serious consequences such as corneal perforation and blindness. It also increases medical expenses and severely reduces the quality of life of patients with dry eye disease. Therefore, the treatment of dry eye syndrome is of great clinical value.

Dry eye disease generally includes two types: aqueous tear deficiency and aqueous tear evaporation, among which Sjogren's syndrome is the primary cause of aqueous tear-deficiency dry eye disease, and there are no effective treatments at present. It is estimated that there are approximately 2 to 4 millions of patients with sicca syndrome in the United States. The secretory function of the lacrimal gland is regulated by the nervous system, including sympathetic nerves, parasympathetic nerves, and higher brain centers. The cause of decreased tear secretion in the sicca syndrome is not very clear. Some researchers consider that it is the progressive infiltration and occupation of the lacrimal gland tissue by inflammatory cells that cause the glands to gradually lose function, resulting in dry eyes. However, recent studies have found that the functional acinar tissue and nerve distribution involved in tear secretion in patients with sicca syndrome are sufficient to maintain normal tear secretion. The decrease in tear secretion is not due to the inflammatory response destroying too much lacrimal gland tissue and leading to insufficient tear secretion, but is related to neurotransmitter release disorder. Pro-inflammatory factors, such as IL-1β, IL-6, and TNF-α, play an important role in the occurrence and development of lacrimal gland inflammation. Some studies have shown that these pro-inflammatory factors can reduce the release of neurotransmitters, such as acetylcholine and norepinephrine, thus blocking neuro-regulated tear secretion, resulting in tear secretion deficiency, and promoting the occurrence of aqueous tear-deficiency dry eye syndrome.

For the purpose of this disclosure, a subject is diagnosed as moderate to severe dry eye when it meets at least one of the following requirements: (1) Eye dryness score (EDS) ≥40 points (visual analogue scale [VAS] of 0-100 points); (2) Break-up time (BUT) <10 s; (3) Schirmer test result <10 mm/5 min; and (4) Corneal fluorescein staining score (ICSS) ≥2 points.

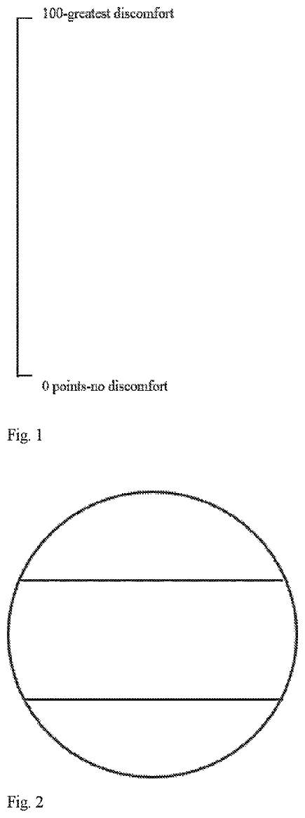

Eye dryness score (EDS) is measured by visual analogue scale (VAS). VAS score for dry eye symptoms is used to assess the severity of the patient's disease. The score includes seven categories of dryness, burning sensation/stinging sensation, itching, foreign body sensation, discomfort, photophobia and pain. Visual analogue scale (VAS) is used for scoring. VAS is a 100 mm long straight line marked with very comfortable and very uncomfortable at both ends. (FIG. 1). Subjects will mark a point on the straight line according to their comfort degree, and the length from the starting point to the mark is the amount of comfort. This method is simple and easy to implement, relatively objective and sensitive. When in use, the graduated side faces away from the subject, and the investigator assigns a score according to the position marked by the subject. At each visit, the subjects are asked in detail about their dry eye symptoms in the last two weeks, and draw their discomfort for each of the seven items. Then the investigator assigns a score according to the position marked by the subject. 0 point represents no discomfort, 100 points represents the greatest discomfort, keep the integer, and the higher the value, the more severe the symptoms.

Break-up time (BUT) determination is used to quantitatively evaluate the stability and quality of the tear film. BUT determination is carried out in the following steps: (1) take out the test paper for corneal and conjunctival staining of the ocular surface, damp the stained part of the test paper with 1-2 drops of sterile intraocular irrigating solution or normal saline, and gently touch the wet part on the subject's cornea and conjunctiva; (2) instruct the subject to blink 3 to 5 times, and record the time from when the eyes were opened naturally after the last blink to the first dark spot on the cornea; (3) measure 3 times with an electronic stopwatch and take the mean value. BUT<10 s means that the break-up time is shortened, which can be diagnose as dry eye; BUT>10 s means normal.

Schirmer test is used to measure basal tear secretion. It is performed in the following steps: (1) the subject is in a dark room, the lower vault of conjunctiva is dripped with anesthetic, the Schirmer test is performed 30 seconds later; (2) measure basal tear secretion by excluding light and filter paper stimulus factor; (3) the examiner folds one end of the standard filter paper 5 mm into a right angle, and places the folded end in the conjunctiva on the lateral ⅓ of the patient's lower eyelid, and the other end hangs outside the eyelid; (4) instruct the patient to lightly close eyes and look up slightly, and blink at will; and (5) gently pull the lower lid and take out the filter paper strip at 5 minutes, and record its length after standing for 2 minutes. Schirmer test result ≥10 mm/5 min is negative. Schirmer test result <10 mm/5 min is positive.

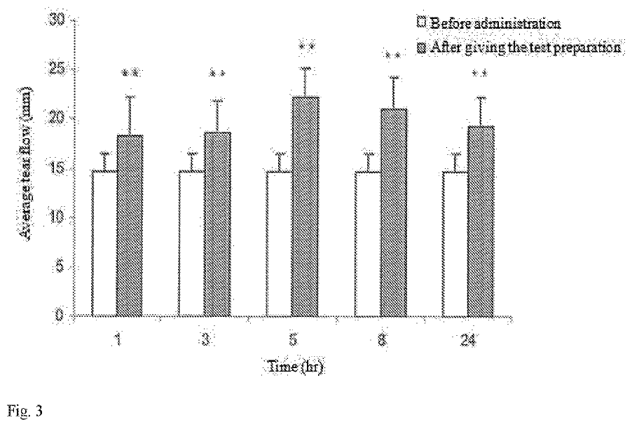

Corneal fluorescein staining score (ICSS) is measured using a 4-point method, wherein only the lower ⅓ of cornea is counted (FIG. 2), and no staining is 0, few/rare punctate lesions is 1 point, discrete and countable lesions (easy to count) is 2 points, lesions too numerous to count, but not coalescent is 3 points and coalescent is 4 points.

Corneal and conjunctival staining and photography is carried out by (1) taking out the test paper for corneal and conjunctival staining of the ocular surface, (2) damping the stained part of the test paper with 1-2 drops of normal saline, (3) gently touching the wet part on the subject's conjunctiva, and then (4) observing and taking pictures of corneal fluorescein staining and conjunctival lissamine green staining.

### Cyclosporine A

Cyclosporines (sometimes referred to as “cyclosporin”) are a class of poly-N-methylated cyclic undecapeptides. There are naturally occurring cyclosporines such as cyclosporine A, and non-natural cyclosporine (“Cs”) derivatives. For example, cyclosporine A (C62 H111 N11 O12), namely Cyclo [[(E)-(2S,3R,4R)-3-hydroxy-4-methyl-2-(methylamino)-6-enoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl], has the following structure:

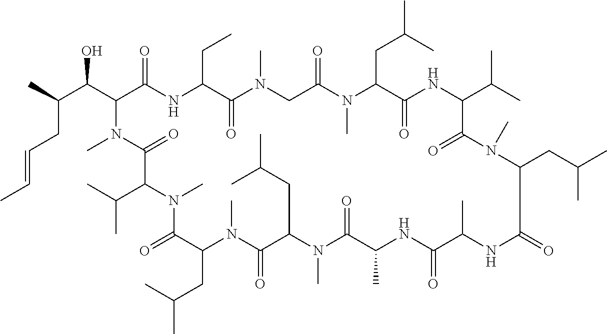 [SMILES: C/C=C/C[C@@H](C)[C@@H](O)C1C(=O)NC(CC)C(=O)N(C)CC(=O)N(C)C(CC(C)C)C(=O)NC(C(C)C)C(=O)N(C)C(CC(C)C)C(=O)NC(C)C(=O)N[C@H](C)C(=O)N(C)C(CC(C)C)C(=O)N(C)C(CC(C)C)C(=O)N(C)C(C(C)C)C(=O)N1C]

Cyclosporine A is an immunosuppressant, which can inhibit the inflammation of the eyes associated with dry eye syndrome, which in turn promotes the secretion of tears. Cyclosporin A can not only prevent T cell activation and the translocation and activation of cytoplasmic transcription factors required for the production of inflammatory factors, but also directly promote the secretion of tears by stimulating nerves. (See, Hiroshi et al., Invest. Ophthalmol. & Vis. Sci. 50(6):2736-2741 (2009); Hiroshi et al., Jpn. J. Ophthalmol. 42:168-173 (1998)) As set forth in U.S. Pat. No. 4,839,342, cyclosporine has been found to be effective in treating immune medicated keratoconjunctivitis sicca (“KCS” or dry eye disease) in a patient suffering therefrom. Some studies have shown that cyclosporine A can promote the secretion of tears through nerve agonism. (See, Hiroshi et al., Invest. Ophthalmol. & Vis. Sci., 50(6):2736-2741 (2009); Hiroshi et al., Jpn. J. Ophthalmol., 42:168-173 (1998).) In addition, cyclosporin A can also inhibit the apoptosis of conjunctival goblet cells. (See, Kunert et al., Arch Ophthalmol., 120(3):330-337 (2002).) Therefore, the mechanism of cyclosporine A in the treatment of the dry eye syndrome may include immunosuppressive action, promotion of tear secretion, and inhibition of conjunctival goblet cell apoptosis.

### Methods

In an aspect, the present disclosure provides a method of treating moderate to severe dry eye disease in a subject in need thereof, comprising administering to the subject an effective amount of cyclosporine A ophthalmic gel.

In some embodiments, the cyclosporine A ophthalmic gel is administered to the subject through conjunctival administration. In some embodiments, the cyclosporine A ophthalmic gel is administered to the subject through ophthalmic administration. In some embodiments, the cyclosporine A ophthalmic gel is administered to the subject through cutaneous administration or percutaneous administration.

In some embodiments, the cyclosporine A ophthalmic gel used in the methods disclosed herein is a cyclosporine A ophthalmic gel as described in the Chinese Patent No. ZL201410033737.3 (published as CN103735495B), which is incorporated by reference in its entirety. In some embodiments, the cyclosporine A ophthalmic gel comprises 0.001-1 wt/o cyclosporine A, 0.1-10 wt/o nonionic surfactant, 0.01-5 wt % aqueous gel substrate, 2-30 wt % wetting agent, water, and pH adjusting agent in an amount that results in a final pH of the cyclosporine A ophthalmic gel being 5.0-8.0, wherein the aqueous gel substrate is sodium carboxymethyl cellulose or carbomer.

Human eyes have a special protective mechanism, so ordinary eye drops will be quickly eliminated resulting in low bioavailability. In consideration of the medication safety and the effective increase of drug exposure, the dosage form developed herein is a gel. The pharmaceutical excipient carbomer can form a uniform, transparent, and stable hydrogel at pH 6-12 with good adhesion, which is suitable for eye administration. Compared with the ophthalmic emulsion, it can prolong the action time of the drug in the eye, and has no greasy nor irritation to the mucous membrane of the eye.

In some embodiments, the subject is characterized in a) Eye dryness score (EDS) ≥40 points (visual analogue scale [VAS] of 0-100 points); b) Break-up time (BUT) <10 s; c) Schirmer test result <10 mm/5 min; and d) Corneal fluorescein staining score (ICSS) ≥2 points.

In some embodiments, the subject does not have an alanine aminotransferase (ALT) level ≥2 times the upper limit of normal, an aspartate aminotransferase (AST) level ≥2 times the upper limit of normal, or a serum creatinine (Cr) level ≥1.5 times the upper limit of normal.

ALT and AST levels are measured in international units per liter (IU/L). In some embodiments, the upper limit of normal of ALT level is about 55 IU/L. In some embodiments, the upper limit of normal of AST level is about 48 IU/L. It is understood that measuring ALT and AST levels are common laboratory tests of blood chemistry, and thus a person skilled in the art is able to select methods and conditions suitable for measuring ALT and AST levels of a subject.

In some embodiments, the upper limit of normal of Cr level is about 1.3 mg/dL for adult men, and 1.1 mg/dL for adult women. It is understood that measuring Cr level is a common laboratory test of blood chemistry, and thus a person skilled in the art is able to select methods and conditions suitable for measuring Cr level of a subject.

In some embodiments, the cyclosporine A ophthalmic gel is administered to the patient once a day. In some embodiments, the cyclosporine A ophthalmic gel is administered to the patient every night before the subject goes to bed. In some embodiments, the cyclosporine A ophthalmic gel is administered to the patient every night within 5 minutes, 10 minutes, 30 minutes, 1 hour, or 2 hours before the subject goes to bed. The high bioavailability of cyclosporine A's gel formulation allows once a day dosing, compared to the conventional twice a day dosing. In some embodiments, one dose of the cyclosporine A ophthalmic gel per day can achieve the effect of two doses of RESTASIS®.

In some embodiments, the effective amount of the cyclosporine A ophthalmic gel is one drop per day. In some embodiments, one drop is 50 μl.

In some embodiments, the concentration of cyclosporine A in the cyclosporine A ophthalmic gel is about 0.05% to about 0.1% by weight. In some embodiments, the concentration of cyclosporine A in the cyclosporine A ophthalmic gel is 0.05% by weight. In some embodiments, the concentration of cyclosporine A in the cyclosporine A ophthalmic gel is 0.05%, 0.06%, 0.07%, 0.08%, 0.09%, or 0.1% by weight.

In some embodiments, the effective amount of cyclosporine A ophthalmic gel administered to the subject per day contains about 0.025 mg cyclosporine A.

In some embodiments, the time window between the administration of the cyclosporine A ophthalmic gel and other drugs is no less than 5 minutes. Time window refers to the interval between the administration of the cyclosporine A ophthalmic gel and other drugs. The administration of the cyclosporine A ophthalmic gel could be either before or after the administration of other drugs.

In some embodiments, after being treated with the method described herein, at day 84 since the treatment starts, the subject's ICSS drops by at least 1 point from the subject's ICSS before treatment.

In some embodiments, the treatment period is up to about 12 weeks. As used herein, the term “treatment period” refers to a session of treatment during which the subject is treated regularly according to a given schedule, for example, once per day. In some embodiments, the subject is treated with the method disclosed herein for about 12 weeks. In some embodiments, the subject is administered the cyclosporine A ophthalmic gel one drop per day for about 12 weeks. As shown in Example 7, the proportion of subjects with at least a 1-point improvement in ICSS from baseline to day 84 was 73.7% (232/315) in the cyclosporine A ophthalmic gel group vs 53.2% (166/312) in the vehicle group (P<0.0001), achieving the study's primary endpoint. The mean changes from baseline in the Schirmer tear test on 84 were significantly higher in the cyclosporine A ophthalmic gel group than in the vehicle group (both P<0.05). The improvement in bioavailability of cyclosporine A in the new gel dosage form makes this short treatment period possible.

In some embodiments, in a treatment period, the onset of efficacy of the cyclosporine A ophthalmic gel starts no more than 30 days since the first administration of the cyclosporine A ophthalmic gel to the subject. In some embodiments, in a treatment period, the onset of efficacy of the cyclosporine A ophthalmic gel starts no more than 14 days since the first administration of the cyclosporine A ophthalmic gel to the subject. In some embodiments, in a treatment period, the onset of efficacy of the cyclosporine A ophthalmic gel starts on day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 since the first administration of the cyclosporine A ophthalmic gel to the subject.

As used herein, “onset of efficacy” is used interchangeably with “onset of action” and “onset of effect.” For the purpose of this application, “onset of efficacy” refers to the first day on which the proportion of subjects with ICSS decreased by ≥1 point was significantly greater in the cyclosporine A ophthalmic gel group than in the vehicle group (p<0.05).

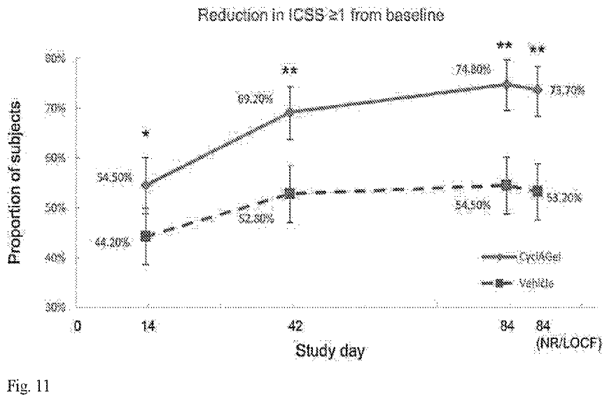

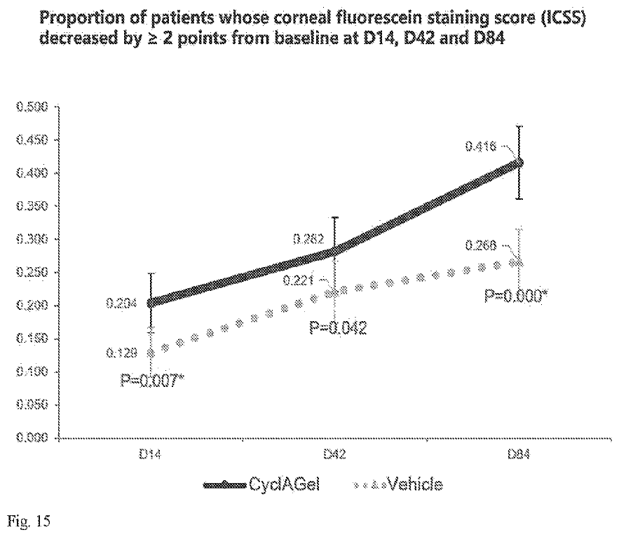

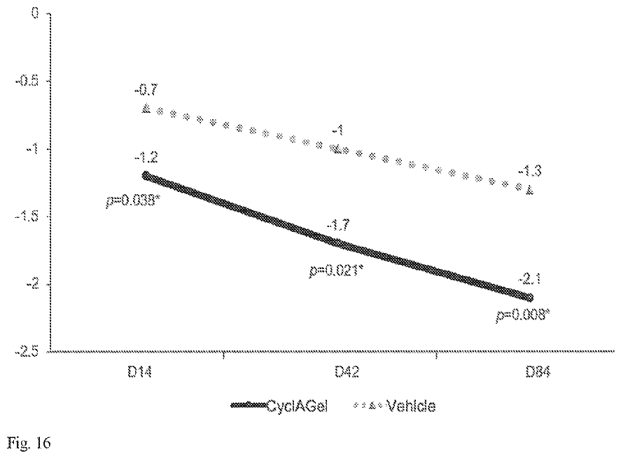

In some embodiments, in a treatment period, the onset of efficacy starts no more than 30 days since the first administration of the cyclosporine A ophthalmic gel to the subject. In some embodiments, in a treatment period, the onset of efficacy starts no more than 14 days since the first administration of the cyclosporine A ophthalmic gel to the subject. As shown in Example 7, the proportion of subjects with at least a 1-point improvement in ICSS from baseline at day 14 was significantly greater in the cyclosporine A ophthalmic gel group than in the vehicle group, with P<0.025. (See FIGS. 11 and 15) Compared with the vehicle group, the decrease of keratoconjunctival staining scores from baseline is also significantly greater in the cyclosporine A ophthalmic gel group than in the vehicle group as early as D14. (FIG. 16) This is a very unexpected result in light of prior cyclosporin A ophthalmic products (such as RESTASIS®) which generally require several months before onset of efficacy. (See Example 8).

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1 Pharmacology of the Cyclosporine A Ophthalmic Gel

Cyclosporine A ophthalmic gel contains 0.05% cyclosporin A.

Generic name: Cyclosporine A Ophthalmic Gel

1.2 Chemical name: Cyclo [[(E)-(2S, 3R, 4R)-3-hydroxy-4-methyl2-(methylamino)-6-enoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl]

English name: Cyclosporine A Ophthalmic Gel

Chinese phonetic alphabet: Huanbaosu A Yanningjiao

### Structural Formula:

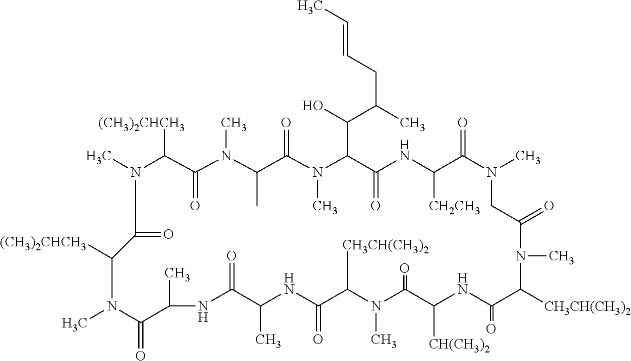

Molecular formula: C62H111N11O12

Molecular weight: 1202.63

Indications: Moderate to severe dry eyes.

Usage and dosage: Once a day, use it every night before going to bed, and put one drop into the affected eye when using.

Adverse reaction: Phase II clinical study results showed that the most common adverse reactions were eye pain (18.18%), foreign body sensation in eyes (3.98%), and blurred vision (2.84%).

### Stability Study

The test samples were tested for high temperature, light, and other influencing factors. The results showed that the related substances increased slightly when the product was at high temperature (60° C. and 40° C.), which was equivalent to that of cyclosporine ophthalmic emulsion (Restasis®), indicating that the product has good thermal stability. After being placed in the light for 10 days, the related substances of the unfilled preparation increased more, indicating that the product is sensitive to light; after being filled with packaging materials, the related substances have no obvious change, indicating that the product is relatively stable to light after being filled.

Three batches of samples (batch numbers: 20130508, 20130509, 20130510) were subjected to accelerated test under simulated marketing packing conditions at a temperature of 30°±2° C. and a relative humidity of 25%±5%. The results showed that after 5 months of accelerated test, the related substances increased slightly, and there was no obvious change in the other indicators. Therefore, it can be preliminarily predicted that the validity period of this product is tentatively set for two years.

Three batches of samples (batch numbers: 20130508, 20130509, 20130510) were subjected to long-term retention sample test under simulated marketing packing conditions at a temperature of 25° C.±2° C. and a relative humidity of 40%±10%. At present, the samples have been retained for 5 months, the increase of related substances was small, and there was no obvious change in the other indicators. The long-term retention sample test is still in progress.

Three batches of samples (batch numbers: 20190501, 20190502, 20190503) were subjected to accelerated test under simulated marketing packing conditions at a temperature of 30°±2° C. and a relative humidity of 35%±5%. The results showed that after 6 months of accelerated test, there was no significant increase in related substances, and there was no obvious change in the other indicators. Therefore, it can be preliminarily predicted that the validity period of this product is tentatively set for two years.

The batch 20190401 and three batches of samples (batch numbers: 20190501, 20190502, 20190503) were subjected to long-term retention sample test under simulated marketing packing conditions at a temperature of 25° C.±2° C. and a relative humidity of 40%±10%. At present, the samples have been retained for 6 months, there was no significant increase in related substances, and there was no obvious change in the other indicators.

### Example 2 Pharmacodynamic Tests

Cyclosporine A is an immunosuppressant. It can inhibit the inflammation of the eyes associated with dry eye disease, which in turn promotes the secretion of tears. However, some studies have shown that, cyclosporine A can promote the secretion of tears through nerve agonism. In addition, cyclosporin A can also inhibit the apoptosis of conjunctival goblet cells. Therefore, the mechanism of cyclosporine A in the treatment of dry eye disease may include immunosuppressive action, promotion of tear secretion, and inhibition of conjunctival goblet cell apoptosis.

In the application materials of cyclosporin A ophthalmic emulsion (Restasis®), a drug efficacy study conducted by Allergan company using KCS (dry eye disease) dogs was provided. In the study, dogs with dry eye disease were given cyclosporine A emulsion (0.05% or higher of 0.2%) twice a day. At both dose levels, the animals showed some significant ophthalmic improvement (although only at 0.2% concentration, the decrease in lymphocytic infiltration of the accessory lacrimal glands and conjunctiva was significant). A small increase in tear production was observed at both dose levels (as measured by the Schirmer Tear Test [STT]), but this increase was not very significant compared to the significant increase observed in studies using other drug products. The vehicle itself showed a delayed effect. There was very little or no difference in STT values between the solvent, 0.05%, and 0.2% cyclosporine A (6 weeks after administration). This study also provided evidence that the lacrimal gland and conjunctival epithelial cell apoptosis was involved in the pathophysiology of dogs with dry eye disease, and treatment with 0.2% cyclosporin A emulsion can reverse some of that (0.05% concentration was not studied). Compared with normal animals, the concentration of cytokine TGF-β1 in the tears of dogs with dry eye disease was significantly increased, and treatment with 0.2% cyclosporin A emulsion can reduce its level. In conclusion, cyclosporin A emulsion exerts a beneficial effect in dogs with dry eye disease, although the histological reduction in lymphocytic infiltration is only apparent at 0.2% concentration treatment.

The results in the above dog model with dry eye disease showed that the 0.05% cyclosporin A emulsion can increase tear secretion, and there was little or no difference in tear secretion increase between 0.2% and 0.05% cyclosporine A emulsion. However, the reduction in lymphocytic infiltration was evident at only 0.2% concentration. Therefore, it is reasonable to believe that the therapeutic effect of cyclosporine A on dry eye disease depends not only on its immunosuppressive action, but also other actions.

Several studies have shown that cyclosporine A can increase tear secretion not only in dogs with dry eye disease, but also in normal dogs/rabbits (this suggests that the effect is independent of immunosuppression and may induce reflex tear secretion through nerve agonism action), and the effect can be seen after only one dose (the effect was dose-dependent, not at low doses, nor in the vehicle group (normal saline, etc.)).

The effect is somewhat similar to hypoglycemic drugs (insulin-stimulating drugs). An important part of the preclinical efficacy experiment of hypoglycemic drugs (insulin-stimulating drugs)(before diabetic animal experiment) is the evaluation of hypoglycemic efficacy in mice with normal blood glucose (C57BL/6 mice are often used).

In view of the difficulties in establishing a dog model with dry eye disease in China, the evaluation ideas of hypoglycemic drugs and the effect of cyclosporin A in promoting tear secretion in normal rabbits, a comparative experiment was conducted on the pharmacodynamics of promoting tear secretion between cyclosporine A ophthalmic gel and cyclosporine A ophthalmic emulsion (Restasis®) in normal rabbits.

A total of 108 common-grade New Zealand white rabbits were used in the pharmacokinetic test. They were divided into two groups, 54 rabbits in each group, half male and half female. The test preparation (cyclosporine A ophthalmic gel, 0.05%) and the reference preparation (Limeda ophthalmic emulsion, 0.05%) were administered to the right eye at a dose of 40 μl/animal. The plasma, tears, conjunctiva, cornea, lacrimal gland and anterior aqueous humor were collected at 20 min, 40 min, 1 hr, 2 hr, 4 hr, 8 hr, 24 hr, 48 hr, and 96 hr after administration, respectively (3 male rabbits and 3 female rabbits at each time point). The tissue was collected from the dosing right eye. Liquid chromatography tandem mass spectrometry (LC-MS/MS) analytical method was used to quantify the concentration of cyclosporine A in plasma and intraocular subdivisions of New Zealand white rabbits. The pharmacokinetic parameters were analyzed according to the non-compartmental model by WinNonlin 6.2 software.

A total of 24 common-grade New Zealand white rabbits were used in the pharmacodynamic test. They were divided into 2 groups, with 12 rabbits in each group, half male and half female. The test preparation (cyclosporine A ophthalmic gel, 0.05%) and the reference preparation (Lianda ophthalmic emulsion, 0.05%) were administered to the right eye in a single dose at a dose of 40 μl/animal. Schirmer tear secretion test was performed on the dosing eye (right eye) before administration and 1 hr, 3 hr, 5 hr, 8 hr, and 24 hr after administration.

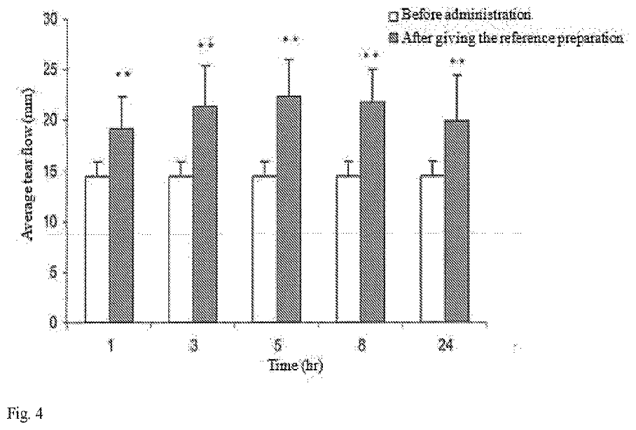

PD (pharmacodynamics) test results (see FIG. 3 and FIG. 2 for details) showed that the tear secretion of New Zealand white rabbits increased significantly at each time point after the two preparations were administered, and reached the highest level 5 hours after the administration. There was a significant difference in tear secretion compared with the baseline level before administration (P<0.01). Compared with the reference preparation, the average amount of tear secretion at each time point induced by the test preparation was slightly lower (24.5%-51% and 34%-54.9/o, respectively), but there was no significant difference (P >0.05). Both cyclosporin A preparations significantly promoted the tear secretion in New Zealand white rabbits, and there was no significant difference between the test preparation and the reference preparation. Therefore, the 0.05% gel preparation and RESTASIS® (cyclosporine A ophthalmic emulsion) from ALLERGAN have basically the same promoting effect in increasing the tear secretion in rabbits.

For the immunosuppressive action of cyclosporine A, the clinical effective concentration of cyclosporine A should be maintained above 150-300 ng/mL when administered systemically. In the PK comparison experiment conducted at the same time, the concentration of cyclosporine A in cornea and conjunctiva of the 0.05% gel preparation was several times higher than that of RESTASIS® (cyclosporine A ophthalmic emulsion) from ALLERGAN. Therefore, it is reasonable to believe that the immunosuppressive action of the 0.05% gel preparation should be stronger than that of ALLERGAN's emulsion preparation.

### Example 3 Pharmacokinetic Tests

### Ophthalmic Pharmacokinetic Data of Cyclosporine A Ophthalmic Emulsion (Restasism®)

ALLERGAN conducted single-dose and multiple-dose pharmacokinetic studies in New Zealand white rabbits and beagle dogs using 31-labeled cyclosporine A at concentrations of 0.05%, 0.2% and 0.4%. The pharmacokinetic study results of a single dose at multiple concentrations in New Zealand white rabbits will be mainly introduced here for comparison with other data (Acheampong et al., Distribution of Cyclosporin A in ocular tissues after administration to albino rabbits and beagle dogs. Current Eye Research, 1999. 18(2): p. 91-103). The details are shown in Table 1:

TABLE 1
Effect of 3 dose concentrations on the penetration of 3H-cyclosporin A
emulsion into eye tissues of New Zealand white rabbits
A. Maximum tissue concentration (Cₘₐₓ)

| | Tissue Cₘₐₓ (ng-eq/g) | | |
|---|---|---|---|
| | Dose concentration a | | |
| Eye tissue | 0.05% b | 0.2% c | 0.4% d |
| Upper conjunctiva | 1320 ± 500 | 2610 ± 290 | 2750 ± 900 |
| Lower conjunctiva | 946 ± 276 | 2180 ± 250 | 2840 ± 1300 |
| Cornea | 873 ± 232 | 2650 ± 910 | 4050 ± 800 |
| Aqueous fluid | 0.838 ± 0.323 | 1.99 ± 0.41 | 4.82 ± 0.85 |
| Iris-ciliary body | 13.7 ± 4.3 | 32.9 ± 16.0 | 85.9 ± 38.8 |
| Lacrimal gland | 6.16 ± 3.69 | 32.7 ± 30.0 | 53.3 ± 43.5 |

a Rabbits are given a single dose of 50 μL 3H-cyclosporin A emulsion;
b, c, d values are shown as the mean ± SD of 5-6 animals.
B. Area under the curve (AUC)

| | Tissue Cₘₐₓ (ng-eq/g) | | |
|---|---|---|---|
| | Dose concentration a | | |
| Eye tissue | 0.05% b | 0.2% c | 0.4% d |
| Upper conjunctiva | 5560 ± 240 | 12400 ± 600 | 15000 ± 1400 |
| Lower conjunctiva | 4690 ± 240 | 9730 ± 670 | 14900 ± 1600 |
| Cornea | 14100 ± 1000 | 43300 ± 3800 | 73300 ± 3300 |
| Aqueous fluid | 12.5 ± 1.3 | 30.4 ± 4.4 | 69.4 ± 8.4 |
| Iris-ciliary body | 246 ± 21 | 675 ± 99 | 1220 ± 80 |
| Lacrimal gland | 64.4 ± 4.3 | 332 ± 39 | 493 ± 41 |

a Rabbits are given a single dose of 50 μL 3H-cyclosporin A emulsion;
b, c, d values are shown as the mean ± SD of 5-6 animals.

The time for 3H-cyclosporine A emulsion to reach peak concentration in the conjunctiva was only 20 minutes; while the time to peak in the cornea was 8 hours, and the concentration decreased slowly thereafter. After 24 hours of administration, the concentrations in the cornea at 0.059, 0.2%, and 0.4% concentrations were 422, 1,410, and 2,810 ng-eq/g, respectively.

Although the drug concentration in eye tissue increased with the increase of dose, the increase of drug concentration in conjunctiva and cornea was smaller than the increase of drug dose.

### Ophthalmic Pharmacokinetic Data of Type A Cyclosporine A Implant

After implanting a type A cyclosporine A implant (investigational product of NIH in the United States) in the episclera 5 mm behind and parallel to the limbus corneae, animals were examined weekly and sacrificed with an overdose of pentobarbital intracardiac injection every 1 to 4 weeks until 6 months of age. The concentration of cyclosporin A in each eye tissue of killed animals was measured. The specific results are shown in Table 2:

TABLE 2
Drug concentration in 6-month eye tissue of cyclosporin A implant

| | Lacrimal | | | | Ciliary | Aqueous | Vitreous |
|---|---|---|---|---|---|---|---|
| | Gland | Conjunctiva | Cornea | Sclera | Body/Iris | Humor | Humor |
| 1 Week | 0.059 ± | 1.872 ± | 1.033 ± | 0.479 ± | 0.227 ± | 0.124 ± | 0.005 ± |
| | 0.0040 | 0.4886 | 0.2826 | 0.2118 | 0.0547 | 0.1156 | 0.0014 |
| 3 Weeks | 0.264 ± | 1.804 ± | 2.792 ± | 0.306 ± | 0.132 ± | 0.116 ± | 0.001 ± |
| | 0.2523 | 0.5263 | 1.0971 | 0.1566 | 0.0497 | 0.1459 | 0.0001 |
| 6 Weeks | 0.077 ± | 1.052 ± | 0.910 ± | 0.570 ± | 0.144 ± | 0.083 ± | 0.004 ± |
| | 0.0401 | 0.3927 | 0.0649 | 0.2962 | 0.0236 | 0.0596 | 0.0004 |
| 3 Months | 0.169 ± | 0.557 ± | 0.418 ± | 0.202 ± | 0.106 ± | 0.117 ± | 0.003 ± |
| | 0.1252 | 0.2820 | 0.1571 | 0.0589 | 0.0766 | 0.1075 | 0.0017 |
| 4 Months | 0.423 ± | 0.591 ± | 0.346 ± | 0.432 ± | 0.198 ± | 0.099 ± | 0.005 ± |
| | 0.5219 | 0.0668 | 0.2070 | 0.0884 | 0.0784 | 0.0639 | 0.0021 |
| 5 Months | 0.123 ± | 0.144 ± | 0.056 ± | 0.163 ± | 0.110 ± | 0.080 ± | 0.003 ± |
| | 0.0057 | 0.0495 | 0.0336 | 0.1065 | 0.133 | 0.0493 | 0.0055 |
| 6 Months | 0.029 ± | 0.098 ± | 0.102 ± | 0.108 ± | 0.049 ± | 0.019 ± | 0.001 ± |
| | 0.0049 | 0.0402 | 0.0364 | 0.0326 | 0.0046 | 0.0225 | 0.0006 |
| Drug | 75.1 ± | 65.7 ± | 65.1 ± | 69.5 ± | 72.3 ± | 63.8 ± | 81.1 ± |
| recovery (%) | 5.78 | 4.19 | 1.96 | 1.75 | 5.64 | 4.41 | 2.98 |

Note:
The drug concentration is expressed in μg/mg (multiplied by 1,000 when converting to μg/g), and the result is expressed as mean ± SD.
Lacrimal gland means lacrimal gland, Conjunctiva means conjunctiva, Cornea means cornea, Sclera means sclera, Ciliary Boday/Iris means ciliary body/iris, Aqueous Humor means aqueous humor, Vitreous Humor means vitreous humor.

The results show that the type A implant can maintain the concentration of cyclosporin A in cornea and conjunctiva tissue at 98 μg/g or more in 6 months, which is more than 100 times higher than that of cyclosporine ophthalmic emulsion Restasis (Restasis®) from Allergan (0.05% concentration).

### Ophthalmic Pharmacokinetic Data of Cyclosporine A Ophthalmic Gel (0.05%)

The pharmacokinetic study of cyclosporine A ophthalmic gel provided herein has been completed in the eyes of New Zealand white rabbits with a single dose, and at the same time it is directly compared with cyclosporine ophthalmic emulsion (Restasis®) from Allergan.

The experimental results showed that the exposure of cyclosporine A in tear, conjunctiva and cornea was larger and the highest in cornea, while the exposure of cyclosporine A in plasma, lacrimal gland and anterior aqueous fluid was below the lower limit of quantitation after the same amount of two preparations were administered to the ocular surface (the 80% lower limit of quantification: 1.00 ng/mL for plasma, 25.0 ng/mL for tear and 11.0 ng/g for other eye tissues), which indicates that cyclosporine A is mainly distributed in the surface tissues of the eyeball after the administration of these two preparations, and basically does not penetrate into the deep tissues and systemic circulation.

The time to peak of the two preparations was consistent in tears, cornea and conjunctiva. In terms of peak concentration, the gel preparation group (2,352 ng/mL, 2,919 ng/g and 3,418 ng/g, respectively) was about 2.99-5.36 times higher than ALLERGAN's emulsion preparation group (786 ng/mL, 545 ng/g and 775 ng/g, respectively). In terms of exposure, the gel preparation group (14,660 hr*ng/mL, 113,654 hr*ng/g and 16,563 hr*ng/g, respectively) was about 1.68-5.25 times that of ALLERGAN's emulsion preparation group (9,672 hr*ng/mL(g), 21,644 hr*ng/mL(g) and 6,321 hr*ng/mL(g), respectively). Therefore, the bioavailability of the gel preparation in cornea, conjunctiva and tears was 1.68 ˜5.25 times higher than ALLERGAN's emulsion preparation (especially in the cornea). At the same time, both preparations could maintain drug concentration above 150-300 ng/mL in the cornea and conjunctival tissues at most time points (within 8 h for conjunctiva and 48 h for cornea). However, the gel preparation maintained the drug concentration for a longer period of time (within 96 h for cornea and 24 h for conjunctiva), and the drug concentration at the corresponding time point was several times higher than ALLERGAN's emulsion preparation. Therefore, the gel preparation should have a better and longer-term immunosuppressive action in the cornea and conjunctiva than ALLERGAN's emulsion preparation. Cₘₐₓ and AUC_{0-96 h}. The results are shown in Table 3.

TABLE 3
Comparison of Cₘₐₓ and AUC₀₋₉₆ₕ results of the two preparations
in various eye tissues, plasma and tears

| | Cmax (ng/ml (g)) | | AUC0-96 h (hr*ng/ml(g)) | |
|---|---|---|---|---|
| | Ophthalmic | Ophthalmic | Ophthalmic | Ophthalmic |
| Eye tissue | gel | emulsion | gel | emulsion |
| Plasma | N/A | N/A | N/A | N/A |
| Lacrimal gland | N/A | N/A | N/A | N/A |
| Aqueous fluid | N/A | N/A | N/A | N/A |
| Tear | 2352 | 786 | 14660 | 9672 |
| Cornea | 2919 | 545 | 113654 | 21644 |
| Conjunctiva | 3418 | 775 | 16563 | 6321 |

Note:
N/A indicates that the value is not available.

### Example 4 Toxicologic Tests

### General Pharmacological Tests and Literatures

In view of the fact that cyclosporine A is widely used for systemic administration in clinic, cyclosporine A should not cause more safety pharmacological risks when used in ophthalmology. In fact, according to the results of pre-clinical PK experiments, the in vivo drug concentration of cyclosporin A ophthalmic gel and cyclosporine ophthalmic emulsion (Restasis®) was much lower than that of cyclosporin A when administered systemically. Therefore, cyclosporin A ophthalmic gel should not cause more safety pharmacological risks.

In addition, the scope of general pharmacological study that can be exempted in accordance with the “Technical Guidelines for General Pharmacological Study of Chemical Drugs”: cyclosporine A ophthalmic gel can be exempted from general pharmacological study for local drugs with low drug concentration in blood or few distribution in other tissues and organs (such as skin and ophthalmic medication).

### Acute Toxicity Tests and Literatures

The acute toxicity studies of cyclosporine A mainly come from reports in the literatures.

A toxicological evaluation literature on cyclosporine A published in 1983 summarized the acute toxicity of cyclosporine A (Ryffel et al., Toxicological evaluation of cyclosporine A. Arch Toxicol, 1983. 53: p. 107-141).

LD50 values 14 days after a single oral and intravenous administration of cyclosporine A are shown in Table 4.

TABLE 4
Acute toxicity in mice, rats and rabbits

| | LD50 (95% confidence interval, | | |
|---|---|---|---|
| | mg/kg) | | |
| Animal species | I.V. | | Oral |
| Mice | 107 | (—) | 2329 (1848-3020) |
| Rats | 25 | (22-29) | 1480 (1105-1997) |

| | | |
|---|---|---|
| Rabbits | >10 | >1000 |

Obvious signs of toxicity appeared immediately after administration, including hyperventilation, drowsiness, muscle spasms and piloerection. In addition, weight loss and diarrhea were observed after oral administration. Autopsies on decedent animals did not reveal any abnormal findings. These results indicate that the acute toxicity of cyclosporine A in mice, rats and rabbits is of low grade intensity.

### Long Term Toxicity Tests and Literatures

The long-term eye toxicity data of two other cyclosporin A ophthalmic preparations (Cyclosporine A ophthalmic emulsion—Restasis®, type A cyclosporine A implant for episclera) is provided herein to illustrate the safety of the 0.05% gel preparation. In addition, a pharmacokinetic comparison test was conducted between the 0.05% gel preparation and cyclosporine ophthalmic emulsion (Restasis®), which served as a bridge to further illustrate the safety of the 0.05% gel preparation.

The eye toxicity tests conducted by Allergan using cyclosporine ophthalmic emulsion (Restasis®) mainly included: Eye toxicity test in New Zealand white rabbits after repeated administration for 3 months and 6 months, and eye toxicity test in beagle dogs after repeated administration for 1 year. Transient, mild eye discomfort and transient, mild conjunctival congestion were observed in New Zealand white rabbits at 3- and 6-month tests (but not in 1-year test of Beagle dogs) in all treatment groups (including the vehicle group). In short, the 0.4% cyclosporin A ophthalmic emulsion can be well tolerated when administered 6 times a day (except for minor local adverse eye reactions).

The eye toxicity tests conducted by NIH using type A cyclosporine A implant included: 6-month eye toxicity test in New Zealand white rabbits and beagle dogs, and 1-year eye toxicity test in beagle dogs (Kim, H., et al., Preclinical Evaluation of a Novel Episcleral Cyclosporine Implant for Ocular Graft-Versus-Host Disease. Investigative Ophthalmology & Visual Science. 46(2); Lee, S. S., et al., A Pharmacokinetic and Safety Evaluation of an Episcleral Cyclosporine Implant for Potential Use in High-Risk Keratoplasty Rejection. Investigative Ophthalmology & Visual Science. 48(5): p. 2023). Only in 2/6 dogs (1-year eye toxicity test in beagle dogs), a mild lymphocytic reaction occurred after 12 months of administration, but no clinically significant inflammation was observed. Therefore, the type A cyclosporin A implant can be well tolerated by rabbits and dogs.

Comprehensive considering the data of the 0.05% gel preparation, cyclosporine ophthalmic emulsion (Restasis®) and type A cyclosporine A implant in the pharmacokinetic data, it can be concluded that type A cyclosporine A implant can be well tolerated by rabbits and dogs when its cyclosporine A exposure in eye tissue is 100 times higher than that of cyclosporine ophthalmic emulsion (Restasis®), and the cyclosporine A exposure in eye tissue of the 0.05% gel preparation is only several times higher than that of cyclosporine ophthalmic emulsion (Restasis®), so the gel preparation described herein and used in the methods described herein should be well tolerated by rabbits and dogs.

### Local Toxicity Tests and Literatures

Eye irritation test of cyclosporine ophthalmic emulsion (Restasis®) was not carried out separately, but integrated into the toxicity test in New Zealand white rabbits with repeated dosing for 3 months (Angelov, O., et al., Preclinical Safety Studies of Cyclosporine Ophthalmic Emulsion. Advances in Experimental Medicine & Biology, 1998. 438: p. 991-995). The results showed that, transient, mild eye discomfort and transient, mild conjunctival congestion were observed in all treatment groups (including the vehicle group). Cyclosporine ophthalmic emulsion (Restasis®) had slight eye irritation.

The cyclosporin A ophthalmic gel (the concentration of cyclosporin A is 0.05%) was used for one-month rabbit eye irritation test. The results showed that the cyclosporine A ophthalmic gel had no irritation to rabbit eyes.

In short, the eye irritation of the cyclosporine ophthalmic emulsion (Restasis®) should be related to its vehicle. The vehicle used in the 0.05% gel preparation is different from that of cyclosporine ophthalmic emulsion (Restasis®), so there is no eye irritation.

### Mutagenicity Tests and Literatures

The mutagenicity studies of cyclosporin A mainly come from reports in the literatures.

A mutagenic evaluation literature on cyclosporine A published in 1982 summarized the mutagenicity of cyclosporine A (Matter, B. E., et al., Genotoxicity evaluation of cyclosporin A, a new immunosuppressive agent. 105(4): p. 257-264). These tests included the Ames test, micronucleus test in mice and Chinese hamsters, chromosome aberration analysis of bone marrow cells in Chinese hamsters (in vivo test), dominant lethal test in mice, and UDS assay (unscheduled DNA synthesis) in spermatocytes and sperm cells of mice.

These experimental results show that cyclosporin A is not mutagenic. Cyclosporine A has no mutagenic effect and has been demonstrated in long-term carcinogenicity studies using mice and rats (these carcinogenicity studies have shown that cyclosporine A has no carcinogenic effect). These findings clearly show that cyclosporin A is not mutagenic.

### Reproductive Toxicity Tests and Literatures

The reproductive toxicity studies of cyclosporine A mainly come from reports in the literatures.

A toxicological evaluation literature on cyclosporine A published in 1983 summarized the reproductive toxicity of cyclosporine A (B. Ryffel, P. Donatsch, and M. Madorin, Toxicological evaluation of cyclosporine A. Arch Toxicol, 1983. 53: p. 107-141). The tests included Phases I, II and Ill of the reproductive toxicity tests.

The results showed that cyclosporine A was not teratogenic, and the harmful effects of increased embryo mortality and developmental retardation were secondary to the toxicity of cyclosporine A to the mother animal.

### Carcinogenicity Tests and Literatures

The carcinogenicity studies of cyclosporin A mainly come from reports in the literatures.

A toxicological evaluation literature on cyclosporine A published in 1983 summarized the carcinogenicity of cyclosporine A (B. Ryffel, P. Donatsch, and M. Madorin, Toxicological evaluation of cyclosporine A. Arch Toxicol, 1983. 53: p. 107-141). The tests included carcinogenicity tests in 78-week mice and 104-week rats.

The results showed that no carcinogenicity of cyclosporine A was found in 78-week mice and 104-week rats.

### Example 5 Comprehensive Analysis and Evaluation of Pharmacology and Toxicology Research Basis for Research Project Selection

Two research projects were conducted: (1) Comparative study on pharmacokinetics and pharmacodynamics of New Zealand rabbits after a single eye administration of two preparations of cyclosporine A; (2) Eye stimulation test of cyclosporine A ophthalmic gel in rabbits. The purpose of these projects is to show that this product is safer compared with Restasis® (cyclosporine A ophthalmic emulsion) from ALLERGAN. In terms of safety, this product does not increase safety concerns, and does not have the eye irritation of Restasis®. In terms of effectiveness, this product does not reduce the effectiveness of the treatment of dry eye syndrome, and may achieve better efficacy. The conduct of pharmacokinetic tests builds a bridge, based on which the safety and effectiveness are compared.

Other toxicity test data are available in the literature or the application materials of RESTASIS®. There is no need to repeat these tests. The pharmacokinetic tests conducted have provided a bridge for safety comparison.

Therefore, the tests that has completed are sufficient and reasonable.

### Comprehensive Assessment

In terms of pharmacokinetics, the 0.05% gel preparation provide a higher cyclosporine A exposure in cornea and conjunctiva (1.68-5.25 times) than that of Restasis® (cyclosporine A ophthalmic emulsion) from ALLERGAN, but it does not increase the cyclosporine A exposure in the whole body and deep eye tissues.

In terms of pharmacodynamic(s), both the 0.05% gel preparation and ALLERGAN's emulsion preparation can significantly promote the tear secretion in normal rabbits, and the promoting effects of the two are similar. Moreover, based on the pharmacokinetic results, the 0.05% gel preparation have greater exposure in the cornea and conjunctiva, and should achieve better anti-inflammatory effects than ALLERGAN's emulsion preparation. In other words, the efficacy of the 0.05% gel preparation in the treatment of dry eye syndrome (2 times/day) may be better than that of ALLERGAN's emulsion preparation (2 times/day), or more likely close to that of ALLERGAN's emulsion preparation (4 times/day) (clinical trials have shown that Restasis®, given four times a day, is more effective than Restasis®, given two times a day, see Ward, J. D., R. L. Kaswan, and C. M. Salisbury, Spontaneous canine keratoconjunctivitis sicca. A useful model for human keratoconjunctivitis sicca: treatment with cyclosporine eye drops. 1989. 107(8): p. 1210).

In terms of safety, although the pharmacokinetic results show that the 0.05% gel preparation provide a higher cyclosporine A exposure in eye tissues than that of ALLERGAN's emulsion preparation, the results of safety and pharmacokinetic evaluation using type A cyclosporine A implant showed that even a 100-fold increase in cyclosporine A exposure would not cause harmful effects on eye tissue. In addition, eye irritation tests further showed that the 0.05% gel preparation did not cause eye irritation as ALLERGAN's emulsion preparation did.

In summary, the 0.05% gel preparation does not raise any new safety concerns compared to ALLERGAN's emulsion preparation, and may not have the eye irritation as ALLERGAN's emulsion preparation. The 0.05% gel preparation may be more effective than ALLERGAN's emulsion preparation at the same dose and frequency. Therefore, the 0.05% gel preparation can not only fill the gap in the absence of cyclosporin A ophthalmic preparations for the treatment of dry eye syndrome in China, but also should be able to have safety and effectiveness advantages compared to ALLERGAN's emulsion preparation.

### Example 6 Previous Clinical Trials

The completed clinical trials of cyclosporine A ophthalmic preparation at home and abroad are introduced below.

### Human Pharmacokinetics

A study on the determination of cyclosporine A concentration in human whole blood by LC-MS/MS and the pharmacokinetics of cyclosporine ophthalmic emulsion in healthy human showed that the linear range of cyclosporine A was 0.2-20.00 μg/L, and the lower limit of quantification was 0.2 μg/L (Wang Shumin, Li Pengfei and Zhao Xiuli, <Determination of Cyclosporin A in Human Whole Blood by LC-MS-MS Method and Pharmacokinetics of Cyclosporin Ophthalmic Emulsion in Healthy Volunteers>. Journal of Chinese Mass Spectrometry Society, 2010. 3(1): p. 53-58). The results of accuracy and precision showed that the inter-day and intra-day variation was less than 15%, and the relative deviation was −12.60%˜12.80%. The extraction recovery of the method was (98.1±4.7)%, showing good stability. The established method was used to determine the blood concentration of 8 Chinese adult volunteers after being given a single dose of 0.025% cyclosporine A ophthalmic emulsion for 1 drop. The concentrations of all whole blood samples were below the detection limit, indicating that the concentration of cyclosporine A was far below the toxic level at the test dose.

### Clinical Trials of Cyclosporine Ophthalmic Emulsion (Restasis®) in the Treatment of Dry Eye Conducted by Allergan

The pivotal phase III studies 002, 003, and 501 were reanalyzed in subgroup at the time of application. (EMA Procedure No. EMEA/H/C/004229/0000, https://www.ema.europa.eu/en/documents/withdrawal-report/withdrawal-assessment-report-restaysis_en.pdf) The following three populations were identified for analysis:

1. ITT population of all patients who have been randomized and have received at least one study drug treatment.

2. Level 2-3 subgroup (moderate to moderately severe) defined by the International Dry Eye Workshop (DEWS). Patients were included in the analysis if they had a corneal staining score of 2-4, a total staining score of 5-9, a Schirmer tear test (anesthesia) score of greater than 2 mm/5 min, and a blurred vision score of less than 3.

3. Level 4 subgroup (patients with severe dry eye) defined by DEWS. Patients were included in the analysis if they had a corneal staining score of 5-6, a total staining score of 10-15, a Schirmer tear test (anesthesia) score of ≤2 mm/5 min, and a blurred vision score of 3-4.

TABLE 5
Number of patients in the reanalyzed subgroup

| | ITT population | | “Level 2-3 subgroup” | |
|---|---|---|---|---|
| Study | CsA 0.05% | Veh | CsA 0.05% | Veh |
| 002 | 135 | 136 | 55 | 61 |
| 003 | 158 | 156 | 53 | 62 |
| 501 | 143 | 150 | 40 | 45 |
| Pooled data | 436 | 442 | 148 | 168 |

Note:
ITT population means the intention-to-treat population; Level 2-3 subgroup” means a Level 2-3 subgroup based on the DEWS grading of dry eyes; pooled data means pooled data.

Table 6 shows the response rates measured in the subgroups (expressed as % responders) and compared with the overall ITT population. All 6-month data were used, even if the visit occurred after the visit window. If a specific indicator was missing after the baseline visit, the patient was not included in the analysis of that indicator.

TABLE 6
Response rate (%) in all patients and subgroups, study 002, 003 and 501

| | All patients | | | Level 2-3 patients | | |
|---|---|---|---|---|---|---|
| | CsA | | | CsA | | |
| Efficacy Variable | 0.05% | Veh | P- value² | 0.05% | Veh | P-value¹ |

Study 002

| | | | | | | |
|---|---|---|---|---|---|---|
| Total corneal and | 3.9 | 2.4 | NS | 7.7 | 1.8 | NS |
| conjunctival staining | | | | | | |
| | n = 129 | n = 126 | | n = 52 | n = 57 | |
| Schirmer tear test with | 17.1 | 10.1 | NS | 25.0 | 13.7 | NS |
| anaesthesia | | | | | | |
| | N = 117 | n = 109 | | n = 48 | n = 51 | |
| Blurred vision | 25.0 | 27.6 | NS | 38.5 | 40.4 | NS |
| | n = 128 | n = 127 | | n = 52 | n = 57 | |

Study 003

| | | | | | | |
|---|---|---|---|---|---|---|
| Total corneal and | 7.9 | 4.8 | NS | 19.2 | 6.9 | NS |
| conjunctival staining | | | | | | |
| | n = 152 | n = 146 | | n = 52 | n = 58 | |
| Schirmer tear test with | 11.7 | 1.5 | <0.001 | 17 | 1.8 | 0.011 |
| anaesthesia | | | | | | |
| | n = 137 | n = 134 | | n = 47 | n = 55 | |
| Blurred vision | 30.9 | 25.5 | NS | 52.9 | 27.6 | 0.007 |
| | n = 152 | n = 145 | | n = 5i | n = 58 | |

Study 501

| | | | | | | |
|---|---|---|---|---|---|---|
| Total corneal and | 3.7 | 1.4 | NS | 7.9 | 0 | NS |
| conjunctival staining | | | | | | |
| | n = 136 | n = 144 | | n = 38 | n = 45 | |
| Schirmer tear test with | 6.3 | 4.7 | NS | 5.9 | 2.5 | NS |
| anaesthesia | | | | | | |
| | n = 127 | n = 129 | | n = 34 | n = 40 | |
| Blurred vision | 40.6 | 42.6 | NS | 60.5 | 47.7 | NS |
| | n = 133 | n = 141 | | n = 38 | n = 44 | |

Pooled data

| | | | | | | |
|---|---|---|---|---|---|---|
| Total corneal and | 5.3 | 2.9 | NS | 12.0 | 3.1 | 0.006 |
| conjunctival staining | | | | | | |
| | n = 417 | n = 416 | | n = 142 | n = 160 | |
| Schirmer tear test with | 11.5 | 5.1 | 0.002 | 17.0 | 6.2 | 0.007 |
| anaesthesia | | | | | | |
| | n = 381 | n = 372 | | n = 129 | n = 146 | |
| Blurred vision | 32.2 | 32.0 | NS | 40.8 | 28.4 | 0.036 |
| | n = 413 | n = 413 | | n = 98 | n = 109 | |

Note:
efficacy variable means effectiveness indicator; CsA means cyclosporine A; Veh means vehicle; total corneal and conjunctival staining means the total corneal and conjunctival staining; schirmer tear test means schirmer tear test; blurred vision means blurred vision.

The results of the subgroup reanalysis showed that the 0.05% cyclosporine A declared by Allergan company significantly improved the signs and symptoms of patients with Level 2-3 (moderate to moderately severe) dry eye.

Of all the completed clinical trials conducted by Allergan, the Phase II and Phase 111 clinical trials in patients with moderate to severe dry eye provided a safety database of 2,310 patients, 1,757 of whom received cyclosporine at least one dose of cyclosporine ophthalmic emulsion (at any concentration). The safety results of these clinical trials showed that cyclosporine ophthalmic emulsion (Restasis®) did not cause systemic toxicity, and the main adverse reactions were eye AEs, such as eye burning, irritation, pain, etc., and there is no evidence that prolonged exposure can cause sensitization.

TABLE 7
Summary of AEs data by body system

| | | | vehicle/0.1% | |
|---|---|---|---|---|
| | | | cyclosporine A | |
| | | | | 6-month |
| | | | | extended |
| | 0.05% | 0.1% | 6-month | period |
| | cyclosporine | cyclosporine | control | (0.1% |
| | A | A | period | cyclosporine |
| Body system | 12 months | 12 months | (vehicle) | A) |
| Preferred term | N = 436 | N = 437 | N = 442 | N = 323 |
| Overall | 127 (29.1) | 150 (34.3) | 93 (21.0) | 44 (13.6) |
| Body as a whole | 7 (1.6) | 8 (1.8) | 8 (1.8) | 2 (0.6) |
| Headache | | | | |
| Cardiovascular | | 2 (0.5) | | |
| system | | | | |
| Digestive system | 4 (0.9) | 1 (0.2) | 1 (0.2) | 1 (0.3) |
| Metabolism and | | | 1 (0.2) | |
| nutrition | | | | |
| Musculoskeletal | 1 (0.2) | | | |
| system | | | | |
| Nervous system | 2 (0.5) | 4 (0.9) | | |
| Respiratory system | 3 (0.7) | 7 (1.6) | 1 (0.2) | 1 (0.3) |
| Skin | 3 (0.7) | 5 (1.1) | 7 (1.6) | |
| Special sense | 118 (27.1) | 145 (33.2) | 85 (19.2) | 41 (12.7) |
| Burning sensation | 74 (17.0) | 74 (16.9) | 29 (6.6) | 21 (6.5) |
| in the eyes | | | | |
| Eye irritation | 13 (3.0) | 10 (2.3) | 7 (1.6) | 5 (1.5) |
| Sensation of | 12 (2.8) | 8 (1.8) | 8 (1.8) | 2 (0.6) |
| foreign body | | | | |
| Conjunctival | 11 (2.5) | 18 (4.1) | 9 (2.0) | 7 (2.2) |
| congestion | | | | |
| Eye pain | 10 (2.3) | 22 (5.0) | 11 (2.5) | 5 (1.5) |
| Eye stinging | 10 (2.3) | 19 (4.3) | 9 (2.0) | 7 (2.2) |
| Eye secretion | 9 (2.1) | 4 (0.9) | 7 (1.6) | 1 (0.3) |
| Photophobia | 9 (2.1) | 7 (1.6) | 3 (0.7) | |
| Eye pruritus | 8 (1.8) | 16 (3.7) | 7 (1.6) | 2 (0.6) |
| Vision disorder | 8 (1.8) | 14 (3.2) | 12 (2.7) | 1 (0.3) |
| Dry eye | 7 (1.6) | 2 (0.5) | 2 (0.5) | |
| Epiphora | 1 (0.2) | 8 (1.8) | 3 (0.7) | 2 (0.6) |
| Urogenital system | | 1 (0.2) | | |

### A Prospective, Multi-Center, Open-Label Clinical Study of 0.05% Cyclosporine Ophthalmic Emulsion (Restasis®8) in the Treatment of Dry Eye in South Korea

The study was conducted at 26 clinical practice sites in South Korea from August 2006 to March 2009 (Byun. Y. S., et al., Cyclosporine 0.05% ophthalmic emulsion for dry eye in Korea: a prospective, multicenter, open-label, surveillance study. Korean J Ophthalmol, 2011. 25(6): p. 369-74). The main inclusion and exclusion criteria were as follows:

Main inclusion criteria: age >18 years; Diagnosis of moderate to severe dry eye disease (based on the standard clinical practice of clinicians); Symptomatic dry eye disease; No improvement after traditional treatments, such as artificial tears, gels, ointments and lacrimal point blocking.

Main exclusion criteria: Systemic or local use of cyclosporine A within the past 90 days; Any lacrimal point blocking expected to be used during the study period; Women who are pregnant, planning to become pregnant or are breastfeeding; Advanced lacrimal gland disorder or dry eye disease caused by goblet cell destruction; Active stage of eye infection; Suspected allergy to any ingredient in the cyclosporine A formula.

In this study, 392 Korean patients with moderate to severe dry eyes were treated with 0.05% cyclosporine ophthalmic emulsion for 3 months. A total of 362 patients completed the study.

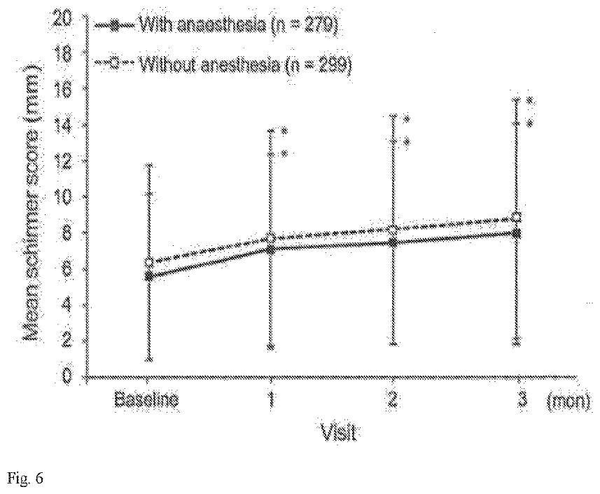

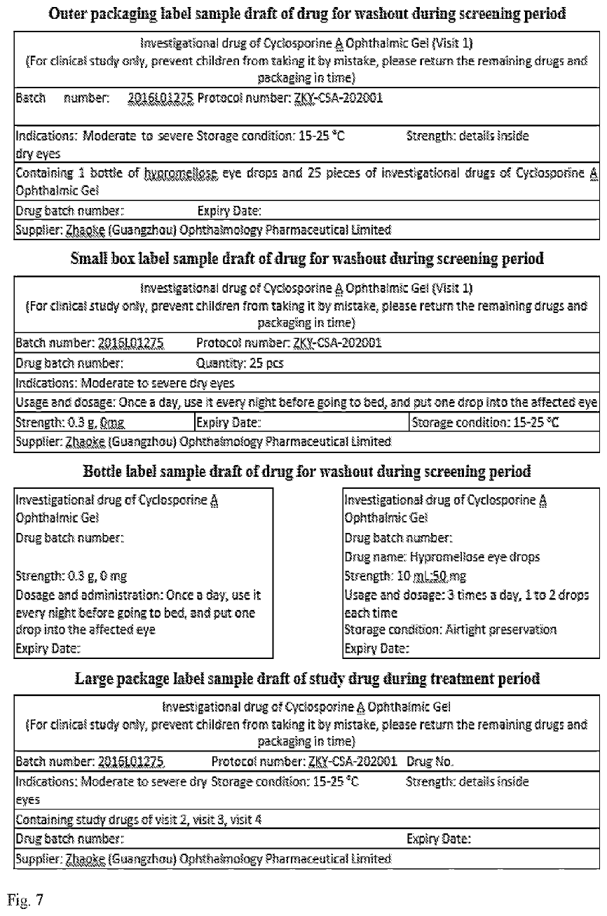

After 3 months of treatment, all eye symptom scores were significantly lower than the baseline value (FIG. 5, FIG. 6); Conjunctiva staining decreased significantly from baseline (P <0.01), as did the use of artificial tears (P<0.0001).

According to the overall evaluation of clinicians, the symptoms of most patients (>50%) were improved by at least 25% to 50%6 from baseline at each visit. The vast majority of patients (72%) were satisfied with the treatment results, and 57.2% of patients reported symptom-free or mild symptoms after treatment. The most common adverse event was eye pain (11.0%). The results of the study proved that in the clinical application of South Korea, 0.05% cyclosporine ophthalmic emulsion has good efficacy and tolerability in the treatment of dry eye.

A Study Comparing Vitamin A (Retinyl Palmitate. Viva®) and 0.05% Cyclosporine Ophthalmic Emulsion (Restasis®) in the Treatment of Dry Eye in South Korea

This study was a prospective, randomized, controlled, and parallel study. A total of 150 patients (300 eyes) diagnosed with dry eye were enrolled (Kim, EC, J. S. Choi, and C. K. Joo, A comparison of vitamin a and cyclosporine a 0.05% eye drops for treatment of dry eye syndrome. Am J Ophthalmol, 2009. 147(2): p. 206-213 e3). 150 patients were randomly divided into 3 groups in a 1:1 ratio, with 50 patients in each group. Group 1 received 0.05% cyclosporine ophthalmic emulsion (Restasis®) for eye droppings, twice daily, group 2 received vitamin A (0.05% retinol palmitate, Viva®) for eye droppings, four times daily, and group 3 (control group) received preservative-free artificial tears (0.5% sodium carboxymethylcellulose, Refresh Plus*) for eye droppings, four times daily.

Inclusion criteria: at least 21 years old; diagnosed as dry eye; difficult to cure with traditional treatments. Schirmer test (without anesthesia) of less than 5 mm/5 min in at least one eye; BUT <5 seconds; mild superficial punctate keratitis, defined as one or more corneal punctate fluorescein staining scores (0 points [none] to 3 points [severe]) per eye. Symptoms of eye irritation were assessed with an ocular surface disease index score of 25 points or more (from 0 to 59 points); the diagnosis of dry eye was based on all the above symptoms and signs.

Exclusion criteria: Within the past six months, patients had a history of any eye disease, including trauma, infection, eye inflammation unrelated to dry eye or surgery; patients receiving treatment that could affect the results of the study; patients suffering from any uncontrollable systemic disease or major disease; patients who are pregnant, breastfeeding, or planning to become pregnant; prior to study completion, patients may be excluded due to adverse events, pregnancy, protocol violations, lack of efficacy or management or personal reasons.

Compared with the control group, vitamin A eye drops and 0.05% cyclosporine ophthalmic emulsion in each group significantly improved the blurred vision, BUT, Schirmer I score and imprint cytology results of patients with dry eye (P<0.05). The results showed that both vitamin A eye drops and 0.05% cyclosporine ophthalmic emulsion were effective in the treatment of dry eyes.

### A Study Evaluating the Efficacy of 0.05% Cyclosporine A Eye Drops in the Treatment of Dry Eye Conducted in China

A total of 60 patients (120 eyes) diagnosed with dry eye disease were enrolled in the study from December 2005 to December 2006 (Feng Qingjun, <Efficacy of Cyclosporine A Eye Drops in the Treatment of Xerophthalmia>. Journal of Practical Medicine, 2007. 23(20): p. 3268-3269). 60 patients were divided into treatment group and control group in a 1:1 ratio, with 30 patients in each group. Patients in the treatment group received 0.05% cyclosporine A eye drops and artificial tears, while patients in the control group only received artificial tears. Efficacy was evaluated after 1 month of continuous medication in both groups, and the changes of Schirmer I test (SIt) and BUT before and after treatment were observed (Table 8). The results showed that compared with the control group, the SIt and BUT of patients in the treatment group were significantly prolonged after treatment, the symptoms of patients were significantly improved, and the clinical efficacy was significantly better than that of the control group (P<0.01). The results showed that 0.05% cyclosporine A eye drops was a safe and effective drug in the treatment of dry eye.

TABLE 8
Comparison of SIt and BUT results between two groups of patients

| | Number of | SIt | BUT |
|---|---|---|---|
| Group | eyes | (mm) | (s) |
| Treatment group | 60 | | |
| Before | | 6.27 ± 1.97 | 6.01 ± 1.69 |
| treatment | | | |
| After treatment | | 9.38 ± 2.56* | 8.85 ± 2.30* |
| Control group | 60 | | |
| Before | | 6.15 ± 1.99 | 6.08 ± 1.77 |
| treatment | | | |
| After treatment | | 6.39 ± 2.02 | 6.14 ± 1.63 |

Note:
*p < 0.01 when compared with the control group.

### A Study Observing the Efficacy of Cyclosporine Eye Drops in the Treatment of Dry Eye Conducted in China

From January to March 2005, a total of 40 patients (80 eyes) diagnosed with dry eye disease were randomly divided into treatment group and control group in a 1:1 ratio, with 20 patients in each group (Hu Jun and Li Guigang, <Treatment of 20 Cases of Xerophthalmia with Cyclosporine Eye Drops>. Herald of Medicine, 2005. 24(12): p. 1116-1117). The treatment group was given 0.05% cyclosporine eye drops twice a day, and Naipuwei eye drops four times a day; the control group was given Naipuwei eye drops four times a day. The efficacy was evaluated after 1 month of continuous treatment. The Schirmer I test, BUT and fluorescein staining results before and after treatment were examined and recorded (Table 9), as well as the degree of improvement in dry eye symptoms after treatment. The results showed that compared with the control group, the Schirmer I test (SIt) and BUT of the treatment group were significantly prolonged, the corneal fluorescein staining grade was reduced, and the clinical symptoms of the patients were significantly improved. The clinical efficacy of the treatment group was significantly better than that of the control group (P<0.01). The results showed that 0.05% cyclosporine eye drops were effective in the treatment of dry eye without obvious adverse reactions.

TABLE 9
Comparison of SIt, BUT and fluorescein staining grading results
between two groups of patients

| | | | | Fluorescein staining | |
|---|---|---|---|---|---|
| | Number | | | grading/eye | |
| | of | | | | Not |
| Item | eyes | SIT/nm | BUT/^{S} | Decreased | decreased |
| Treatment | 40 | | | | |
| group | | | | | |
| Before | | 6.30 ± 2.01 | 5.96 ± 1.78 | | |
| treatment | | | | | |
| After | | 10.15 ± 2.94*¹ | 8.77 ± 2.22 | 30*¹ | 10*¹ |
| treatment | | | | | |
| Control | 40 | | | | |
| group | | | | | |
| Before | | 6.11 ± 1.98 | 6.10 ± 1.83 | | |
| treatment | | | | | |
| After | | 6.35 ± 2.05 | 6.17 ± 1.87 | 11 | 29 |
| treatment | | | | | |

Note:
*¹P < 0.01 when compared with the control group after treatment

### Example 6 Phase II Clinical Study of Cyclosporin A Ophthalmic Gel in the Treatment of Moderate to Severe Tear Deficiency Dry Eye Completed

From December 2017 to November 2019, a multi-center, randomized, single-blind, positive-controlled, dose-finding phase II clinical study was conducted on the effectiveness and safety of cyclosporine A ophthalmic gel in the treatment of patients with moderate to severe tear deficiency dry eyes in 13 study sites including Zhongshan Ophthalmic Center, Sun Yat-sen University, involving 240 subjects. They were randomly assigned to groups A, B, C and D according to 1:1:1:1. Group A was given cyclosporine A ophthalmic gel (0.05%/qD), group B was given cyclosporine A ophthalmic gel (0.05%/B.I.D.), group C was given cyclosporine A ophthalmic gel (0.1%/q.D.), and group D was given cyclosporine ophthalmic emulsion (0.05%/B.ID).

The primary efficacy endpoint of the study was the mean change in EDS scores (in point) from baseline at 12 weeks of treatment.

In FAS, the mean change of EDS score from baseline at 12 weeks of treatment was −29.17±23.77 in test group A, −32.07±21.77 in test group B, −29.75±19.72 in test group C, and −27.26±24.95 in control group. Within the group, the 12-week EDS scores of each group were lower than the baseline, and the difference was statistically significant (p<0.05). Covariance analysis was used to correct baseline for inter-group comparison. The differences between test groups (A, B, C) and control group (D) were as follows: −1.86 (−11.32˜7.61), −3.93 (−13.44˜5.58), −3.01 (−12.44˜6.41), among all groups had no statistical significance (P >0.05), and there was no statistically significant difference between the groups (p>0.05). Each test group (A, B, C) had a better effect trend than the control group (D).

TABLE 10
Comparison of changes in EDS scores from baseline at 12 weeks after medication in four groups (FAS)

| | | Group A | Group B | Group C | Group D | Statistics | P value |
|---|---|---|---|---|---|---|---|
| Week | N(Miss) | 58(0) | 57(0) | 59(0) | 58(0) | F | 0.7841 |
| 12 VS. | Mean ± SD | −29.17 ± | −32.07 ± | −29.75 ± | −27.26 ± | | |
| baseline | | 23.77 | 21.77 | 19.72 | 24.95 | | |
| | 95% CI | (−35.42, | (−37.85, | (−34.89, | (−33.82, | | |
| | | −22.92) | −26.29) | −24.61) | −20.70) | | |
| | Min~Max | −90.00~21.00 | −71.00~14.00 | −64.00~20.00 | −85.00~45.00 | | |
| | Intra-group | −9.348 | −11.12 | −11.58 | −8.320 | | |
| | comparison | | | | | | |
| | of t | | | | | | |
| | Intra-group | 0.0000 | 0.0000 | 0.0000 | 0.0000 | | |
| | comparison | | | | | | |

In PPS, the mean change of EDS score from baseline at 12 weeks of treatment was −28.75±24.14 in test group A, −34.04±21.16 in test group B, −31.23±19.87 in test group C, and −29.14±25.59 in control group. Within the group, the 12-week EDS scores of each group were lower than the baseline, and the difference was statistically significant (p<0.05). Covariance analysis was used to correct baseline for inter-group comparison. The differences between test groups (A, B, C) and control group (D) were as follows: −0.27 (−10.25˜9.7), −3.84 (−13.93˜6.25), −3.20 (−13.18˜6.78), among all groups had no statistical significance (P >0.05), and there was no statistically significant difference between the groups (p>0.05).

TABLE 11
Comparison of changes in EDS scores from baseline at 12 weeks after medication in four groups (PPS)

| | | Group A | Group B | Group C | Group D | Statistics | P value |
|---|---|---|---|---|---|---|---|
| Week | N(Miss) | 51(0) | 48(0) | 52(0) | 50(0) | F | 0.8012 |
| 12 VS. | Mean ± SD | −28.92 ± | −34.42 ± | −31.23 ± | −29.14 ± | | |
| baseline | | 24.34 | 21.49 | 19.87 | 25.59 | | |
| | 95% CI | (−35.77, | (−40.66, | (−36.76, | (−36.41, | | |
| | | −22.07) | −28.18) | −25.70) | −21.87) | | |
| | Min~Max | −90.00~21.00 | −71.00~14.00 | −64.00~20.00 | −85.00~45.00 | | |
| | Intra-group | −8.485 | −11.09 | −11.33 | −8.051 | | |
| | comparison | | | | | | |
| | of t | | | | | | |
| | Intra-group | 0.0000 | 0.0000 | 0.0000 | 0.0000 | | |
| | comparison | | | | | | |
| | of P | | | | | | |

Adverse events in this trial occurred in 18 system organ categories. The classification of adverse events according to system organ categories was shown in Table 12.

TABLE 12
Occurrence of adverse events in each system in the four groups (SS)

| | | Group A | | | Group B | | | | | | Group D | | |
|---|---|---|---|---|---|---|---|---|---|---|---|---|---|
| | | | Number | | | Number | | Group C | | | | Number | |
| | | No. of | of | Per- | No. of | of | Per- | No. of | Number | Per- | No. of | of | Per- |
| SOC name | PT name | cases | subjects | centage | cases | subjects | centage | cases | of | centage | cases | subjects | centage |
| Metabolism | | 0 | 0 | 0.00% | 2 | 2 | 3.45% | 1 | 1 | 1.69% | 2 | 2 | 3.39% |
| and | | | | | | | | | | | | | |
| nutrition | | | | | | | | | | | | | |
| disorders | | | | | | | | | | | | | |
| | Hyper- | 0 | 0 | 0.00% | 2 | 2 | 3.45% | 1 | 1 | 1.69% | 2 | 2 | 3.39% |
| | lipidaemia | | | | | | | | | | | | |
| Ear and | | 0 | 0 | 0.00% | 1 | 1 | 1.72% | 2 | 1 | 1.69% | 0 | 0 | 0.00% |
| labyrinth | | | | | | | | | | | | | |
| disorders | | | | | | | | | | | | | |
| | Tinnitus | 0 | 0 | 0.00% | 0 | 0 | 0.00% | 1 | 1 | 1.69% | 0 | 0 | 0.00% |
| | Neurosensory | 0 | 0 | 0.00% | 0 | 0 | 0.00% | 1 | 1 | 1.69% | 0 | 0 | 0.00% |
| | deafness | | | | | | | | | | | | |
| | Sudden hearing | 0 | 0 | 0.00% | 1 | 1 | 1.72% | 0 | 0 | 0.00% | 0 | 0 | 0.00% |
| | loss | | | | | | | | | | | | |
| Hepato- | | 0 | 0 | 0.00% | 0 | 0 | 0.00% | 1 | 1 | 1.69% | 2 | 2 | 3.39% |
| biliary | | | | | | | | | | | | | |
| disorders | | | | | | | | | | | | | |
| | Hepatic | 0 | 0 | 0.00% | 0 | 0 | 0.00% | 0 | 0 | 0.00% | 2 | 2 | 3.39% |
| | function | | | | | | | | | | | | |
| | abnormalities | | | | | | | | | | | | |
| | Hepatic | 0 | 0 | 0.00% | 0 | 0 | 0.00% | 1 | 1 | 1.69% | 0 | 0 | 0.00% |
| | steatosis | | | | | | | | | | | | |
| Infections | | 13 | 12 | 20.34% | 14 | 10 | 17.24% | 6 | 5 | 8.47% | 9 | 7 | 11.86% |
| and | | | | | | | | | | | | | |
| infestations | | | | | | | | | | | | | |
| | Nasophar- | 5 | 4 | 6.78% | 4 | 3 | 5.17% | 3 | 2 | 3.39% | 2 | 1 | 1.69% |
| | yngitis | | | | | | | | | | | | |
| | Sinusitis | 1 | 1 | 1.69% | 0 | 0 | 0.00% | 0 | 0 | 0.00% | 0 | 0 | 0.00% |
| | Tonsillitis | 0 | 0 | 0.00% | 0 | 0 | 0.00% | 1 | 1 | 1.69% | 0 | 0 | 0.00% |
| | Chronic | 0 | 0 | 0.00% | 1 | 1 | 1.72% | 0 | 0 | 0.00% | 0 | 0 | 0.00% |
| | tonsillitis | | | | | | | | | | | | |
| | Urinary tract | 5 | 5 | 8.47% | 5 | 5 | 8.62% | 0 | 0 | 0.00% | 2 | 2 | 3.39% |
| | infection | | | | | | | | | | | | |
| | Upper | 2 | 2 | 3.39% | 3 | 3 | 5.17% | 1 | 1 | 1.69% | 4 | 4 | 6.78% |
| | respiratory | | | | | | | | | | | | |
| | tract | | | | | | | | | | | | |
| | infection | | | | | | | | | | | | |
| | Pulpitis | 0 | 0 | 0.00% | 0 | 0 | 0.00% | 1 | 1 | 1.69% | 1 | 1 | 1.69% |
| | Bronchitis | 0 | 0 | 0.00% | 1 | 1 | 1.72% | 0 | 0 | 0.00% | 0 | 0 | 0.00% |
| Inves- | | 7 | 3 | 5.08% | 4 | 4 | 6.90% | 7 | 5 | 8.47% | 2 | 2 | 3.39% |
| tigations | | | | | | | | | | | | | |
| | White blood | 2 | 2 | 3.39% | 1 | 1 | 1.72% | 1 | 1 | 1.69% | 0 | 0 | 0.00% |
| | cell count | | | | | | | | | | | | |
| | increased | | | | | | | | | | | | |
| | Alanine | 0 | 0 | 0.00% | 0 | 0 | 0.00% | 0 | 0 | 0.00% | 1 | 1 | 1.69% |
| | amino- | | | | | | | | | | | | |
| | transferase | | | | | | | | | | | | |
| | increased | | | | | | | | | | | | |
| | Monocyte | 1 | 1 | 1.69% | 0 | 0 | 0.00% | 0 | 0 | 0.00% | 0 | 0 | 0.00% |
| | count | | | | | | | | | | | | |
| | increased | | | | | | | | | | | | |
| | Lymphocyte | 1 | 1 | 1.69% | 0 | 0 | 0.00% | 0 | 0 | 0.00% | 0 | 0 | 0.00% |
| | count | | | | | | | | | | | | |
| | Protein | 0 | 0 | 0.00% | 0 | 0 | 0.00% | 1 | 1 | 1.69% | 0 | 0 | 0.00% |
| | urine | | | | | | | | | | | | |
| | present | | | | | | | | | | | | |
| | Red blood | 1 | 1 | 1.69% | 1 | 1 | 1.72% | 1 | 1 | 1.69% | 0 | 0 | 0.00% |
| | cells urine | | | | | | | | | | | | |
| | positive | | | | | | | | | | | | |
| | Glucose | 0 | 0 | 0.00% | 0 | 0 | 0.00% | 1 | 1 | 1.69% | 0 | 0 | 0.00% |
| | urine | | | | | | | | | | | | |
| | present | | | | | | | | | | | | |
| | Blood urine | 0 | 0 | 0.00% | 0 | 0 | 0.00% | 1 | 1 | 1.69% | 0 | 0 | 0.00% |
| | present | | | | | | | | | | | | |
| | Eosinophil | 1 | 1 | 1.69% | 0 | 0 | 0.00% | 0 | 0 | 0.00% | 0 | 0 | 0.00% |
| | percentage | | | | | | | | | | | | |
| | decreased | | | | | | | | | | | | |
| | ECG abnormal | 0 | 0 | 0.00% | 1 | 1 | 1.72% | 1 | 1 | 1.69% | 0 | 0 | 0.00% |
| | Blood | 0 | 0 | 0.00% | 0 | 0 | 0.00% | 1 | 1 | 1.69% | 0 | 0 | 0.00% |
| | bilirubin | | | | | | | | | | | | |
| | increased | | | | | | | | | | | | |
| | Blood | 0 | 0 | 0.00% | 0 | 0 | 0.00% | 0 | 0 | 0.00% | 1 | 1 | 1.69% |
| | glucose | | | | | | | | | | | | |
| | increased | | | | | | | | | | | | |
| | Blood | 0 | 0 | 0.00% | 1 | 1 | 1.72% | 0 | 0 | 0.00% | 0 | 0 | 0.00% |
| | pressure | | | | | | | | | | | | |
| | increased | | | | | | | | | | | | |
| | Neutrophil | 1 | 1 | 1.69% | 0 | 0 | 0.00% | 0 | 0 | 0.00% | 0 | 0 | 0.00% |
| | count | | | | | | | | | | | | |
| Nervous | | 1 | 1 | 1.69% | 2 | 2 | 3.45% | 4 | 4 | 6.78% | 0 | 0 | 0.00% |
| system | | | | | | | | | | | | | |
| disorders | | | | | | | | | | | | | |
| | Carotid | 0 | 0 | 0.00% | 0 | 0 | 0.00% | 1 | 1 | 1.69% | 0 | 0 | 0.00% |
| | arterio- | | | | | | | | | | | | |
| | sclerosis | | | | | | | | | | | | |
| | Insomnia | 0 | 0 | 0.00% | 1 | 1 | 1.72% | 3 | 3 | 5.08% | 0 | 0 | 0.00% |
| | Headache | 1 | 1 | 1.69% | 1 | 1 | 1.72% | 0 | 0 | 0.00% | 0 | 0 | 0.00% |
| Injury, | | 1 | 1 | 1.69% | 0 | 0 | 0.00% | 2 | 2 | 3.39% | 1 | 1 | 1.69% |
| poisoning | | | | | | | | | | | | | |
| and | | | | | | | | | | | | | |
| procedural | | | | | | | | | | | | | |
| compli- | | | | | | | | | | | | | |
| cations | | | | | | | | | | | | | |
| | Ligament | 0 | 0 | 0.00% | 0 | 0 | 0.00% | 0 | 0 | 0.00% | 1 | 1 | 1.69% |
| | sprain | | | | | | | | | | | | |
| | Injury | 1 | 1 | 1.69% | 0 | 0 | 0.00% | 0 | 0 | 0.00% | 0 | 0 | 0.00% |
| Musculo- | | 1 | 1 | 1.69% | 2 | 2 | 3.45% | 2 | 2 | 3.39% | 2 | 2 | 3.39% |
| skeletal and | | | | | | | | | | | | | |
| connective | | | | | | | | | | | | | |
| tissue | | | | | | | | | | | | | |
| disorders | | | | | | | | | | | | | |
| | Back pain | 0 | 0 | 0.00% | 0 | 0 | 0.00% | 0 | 0 | 0.00% | 1 | 1 | 1.69% |
| | Bone formation | 0 | 0 | 0.00% | 0 | 0 | 0.00% | 1 | 1 | 1.69% | 0 | 0 | 0.00% |
| | increased | | | | | | | | | | | | |
| | Arthralgia | 1 | 1 | 1.69% | 0 | 0 | 0.00% | 0 | 0 | 0.00% | 0 | 0 | 0.00% |
| | Musculo- | 0 | 0 | 0.00% | 1 | 1 | 1.72% | 0 | 0 | 0.00% | 0 | 0 | 0.00% |
| | skeletal | | | | | | | | | | | | |
| | discomfort | | | | | | | | | | | | |
| | Neck pain | 0 | 0 | 0.00% | 0 | 0 | 0.00% | 0 | 0 | 0.00% | 1 | 1 | 1.69% |
| | Soft tissue | 0 | 0 | 0.00% | 0 | 0 | 0.00% | 1 | 1 | 1.69% | 0 | 0 | 0.00% |
| | disorder | | | | | | | | | | | | |
| | Limb | 0 | 0 | 0.00% | 1 | 1 | 1.72% | 0 | 0 | 0.00% | 0 | 0 | 0.00% |
| | discomfort | | | | | | | | | | | | |
| Respiratory, | | 2 | 2 | 3.39% | 2 | 2 | 3.45% | 2 | 2 | 3.39% | 1 | 1 | 1.69% |
| thoracic and | | | | | | | | | | | | | |
| mediastinal | | | | | | | | | | | | | |
| disorders | | | | | | | | | | | | | |
| | Epistaxis | 1 | 1 | 1.69% | 0 | 0 | 0.00% | 0 | 0 | 0.00% | 0 | 0 | 0.00% |
| | Pneumonitis 0 | 0 | 0 | 0.00% | 0 | 0 | 0.00% | 0 | 0 | 0.00% | 1 | 1 | 1.69% |
| | Laryngeal | 1 | 1 | 1.69% | 0 | 0 | 0.00% | 0 | 0 | 0.00% | 0 | 0 | 0.00% |
| | pain 1 | | | | | | | | | | | | |
| | Respiratory | 0 | 0 | 0.00% | 0 | 0 | 0.00% | 1 | 1 | 1.69% | 0 | 0 | 0.00% |
| | tract | | | | | | | | | | | | |
| | haemorrhage | | | | | | | | | | | | |
| | Cough | 0 | 0 | 0.00% | 2 | 2 | 3.45% | 1 | 1 | 1.69% | 0 | 0 | 0.00% |
| Immune | | 0 | 0 | 0.00% | 1 | 1 | 1.72% | 1 | 1 | 0% | 1 | 1 | 1.69% |
| system | | | | | | | | | | | | | |
| disorders | | | | | | | | | | | | | |
| | Hyper- | 0 | 0 | 0.00% | 0 | 0 | 0.00% | 1 | 1 | 1.69% | 0 | 0 | 0.00% |
| | sensitivity | | | | | | | | | | | | |
| | Allergic | 0 | 0 | 0.00% | 1 | 1 | 1.72% | 0 | 0 | 0.00% | 0 | 0 | 0.00% |
| | rhinitis | | | | | | | | | | | | |
| | Papular | 0 | 0 | 0.00% | 0 | 0 | 0.00% | 0 | 0 | 0.00% | 1 | 1 | 1.69% |
| | urticaria | | | | | | | | | | | | |
| Endocrine | | 0 | 0 | 0.00% | 0 | 0 | 0.00% | 1 | 1 | 1.69% | 0 | 0 | 0.00% |
| disorders | | | | | | | | | | | | | |
| | Thyroid mass | 0 | 0 | 0.00% | 0 | 0 | 0.00% | 1 | 1 | 1.69% | 0 | 0 | 0.00% |
| Skin and | | 0 | 0 | 0.00% | 1 | 1 | 1.72% | 0 | 0 | 0.00% | 2 | 2 | 3.39% |
| subcutaneous | | | | | | | | | | | | | |
| tissue | | | | | | | | | | | | | |
| disorders | | | | | | | | | | | | | |
| | Erythema | 0 | 0 | 0.00% | 0 | 0 | 0.00% | 0 | 0 | 0.00% | 1 | 1 | 1.69% |
| | Dermatitis | 0 | 0 | 0.00% | 1 | 1 | 1.72% | 0 | 0 | 0.00% | 0 | 0 | 0.00% |
| | Pruritus | 0 | 0 | 0.00% | 0 | 0 | 0.00% | 0 | 0 | 0.00% | 1 | 1 | 1.69% |
| General | | 1 | 1 | 1.69% | 0 | 0 | 0.00% | 0 | 0 | 0.00% | 3 | 3 | 5.08% |
| disorders | | | | | | | | | | | | | |
| and admin- | | | | | | | | | | | | | |
| istration | | | | | | | | | | | | | |
| site | | | | | | | | | | | | | |
| conditions | | | | | | | | | | | | | |
| | Fever | 1 | 1 | 1.69% | 0 | 0 | 0.00% | 0 | 0 | 0.00% | 3 | 3 | 5.08% |
| Renal and | | 0 | 0 | 0.00% | 0 | 0 | 0.00% | 1 | 1 | 1.69% | 0 | 0 | 0.00% |
| urinary | | | | | | | | | | | | | |
| disorders | | | | | | | | | | | | | |
| | Urinary stone | 0 | 0 | 0.00% | 0 | 0 | 0.00% | 1 | 1 | 1.69% | 0 | 0 | 0.00% |
| Gastro- | | 3 | 3 | 5.08% | 2 | 2 | 3.45% | 8 | 7 | 11.86% | 4 | 4 | 6.78% |
| intestinal | | | | | | | | | | | | | |
| disorders | | | | | | | | | | | | | |
| | Constipation | 0 | 0 | 0.00% | 0 | 0 | 0.00% | 0 | 0 | 0.00% | 2 | 2 | 3.39% |
| | Non-infectious | 0 | 0 | 0.00% | 0 | 0 | 0.00% | 1 | 1 | 1.69% | 0 | 0 | 0.00% |
| | gingivitis | | | | | | | | | | | | |
| | Abdominal | 0 | 0 | 0.00% | 1 | 1 | 1.72% | 1 | 1 | 1.69% | 0 | 0 | 0.00% |
| | discomfort | | | | | | | | | | | | |
| | Pericoronitis | 0 | 0 | 0.00% | 0 | 0 | 0.00% | 0 | 0 | 0.00% | 1 | 1 | 1.69% |
| | Chronic | 1 | 1 | 1.69% | 0 | 0 | 0.00% | 1 | 1 | 1.69% | 0 | 0 | 0.00% |
| | gastritis | | | | | | | | | | | | |
| | Pulpitis | 0 | 0 | 0.00% | 0 | 0 | 0.00% | 1 | 1 | 1.69% | 0 | 0 | 0.00% |
| | Toothache | 1 | 1 | 1.69% | 1 | 1 | 1.72% | 2 | 2 | 3.39% | 0 | 0 | 0.00% |
| | Pharyngitis | 1 | 1 | 1.69% | 0 | 0 | 0.00% | 2 | 2 | 3.39% | 1 | 1 | 1.69% |
| Cardiac | | 0 | 0 | 0.00% | 1 | 1 | 1.72% | 0 | 0 | 0.00% | 4 | 4 | 6.78% |
| disorders | | | | | | | | | | | | | |
| | Extrasystoles | 0 | 0 | 0.00% | 1 | 1 | 1.72% | 0 | 0 | 0.00% | 0 | 0 | 0.00% |
| | ventricular | | | | | | | | | | | | |
| | Dizziness | 0 | 0 | 0.00% | 0 | 0 | 0.00% | 0 | 0 | 0.00% | 3 | 3 | 5.08% |
| | Cardiac | 0 | 0 | 0.00% | 0 | 0 | 0.00% | 0 | 0 | 0.00% | 1 | 1 | 1.69% |
| | disorder | | | | | | | | | | | | |
| Vascular | | 2 | 2 | 3.39% | 0 | 0 | 0.00% | 2 | 2 | 3.39% | 0 | 0 | 0.00% |
| disorders | | | | | | | | | | | | | |
| | Hypotension | 1 | 1 | 1.69% | 0 | 0 | 0.00% | 0 | 0 | 0.00% | 0 | 0 | 0.00% |
| | Hypertension | 0 | 0 | 0.00% | 0 | 0 | 0.00% | 2 | 2 | 3.39% | 0 | 0 | 0.00% |
| | Peripheral | 1 | 1 | 1.69% | 0 | 0 | 0.00% | 0 | 0 | 0.00% | 0 | 0 | 0.00% |
| | ischemia | | | | | | | | | | | | |
| Eye | | 35 | 19 | 32.20% | 36 | 24 | 41.38% | 41 | 21 | 35.59% | 29 | 23 | 38.98% |
| disorders | | | | | | | | | | | | | |
| | Dry eye | 4 | 3 | 5.08% | 3 | 3 | 5.17% | 5 | 4 | 6.78% | 1 | 1 | 1.69% |
| | Photoelectric | 0 | 0 | 0.00% | 0 | 0 | 0.00% | 2 | 1 | 1.69% | 0 | 0 | 0.00% |
| | conjunctivitis | | | | | | | | | | | | |
| | Keratopathy | 1 | 1 | 1.69% | 2 | 2 | 3.45% | 5 | 3 | 5.08% | 1 | 1 | 1.69% |
| | Corneal | 0 | 0 | 0.00% | 1 | 1 | 1.72% | 0 | 0 | 0.00% | 0 | 0 | 0.00% |
| | neovascu- | | | | | | | | | | | | |
| | larisation | | | | | | | | | | | | |
| | Keratitis | 0 | 0 | 0.00% | 1 | 1 | 1.72% | 0 | 0 | 0.00% | 0 | 0 | 0.00% |
| | Conjunctival | 0 | 0 | 0.00% | 1 | 1 | 1.72% | 0 | 0 | 0.00% | 0 | 0 | 0.00% |
| | deposit | | | | | | | | | | | | |
| | Conjunctival | 3 | 2 | 3.39% | 1 | 1 | 1.72% | 1 | 1 | 1.69% | 0 | 0 | 0.00% |
| | congestion | | | | | | | | | | | | |
| | Lacrimation | 1 | 1 | 1.69% | 1 | 1 | 1.72% | 1 | 1 | 1.69% | 0 | 0 | 0.00% |
| | increased | | | | | | | | | | | | |
| | Hypoaesthesia | 1 | 1 | 1.69% | 0 | 0 | 0.00% | 0 | 0 | 0.00% | 0 | 0 | 0.00% |
| | eye | | | | | | | | | | | | |
| | Visual | 0 | 0 | 0.00% | 1 | 1 | 1.72% | 0 | 0 | 0.00% | 0 | 0 | 0.00% |
| | impairment | | | | | | | | | | | | |
| | Asthenopia | 1 | 1 | 1.69% | 0 | 0 | 0.00% | 3 | 2 | 3.39% | 0 | 0 | 0.00% |
| | Blurred vision | 3 | 3 | 5.08% | 2 | 2 | 3.45% | 4 | 3 | 5.08% | 5 | 5 | 8.47% |
| | Photophobia | 4 | 4 | 6.78% | 3 | 3 | 5.17% | 0 | 0 | 0.00% | 0 | 0 | 0.00% |
| | Ocular | 0 | 0 | 0.00% | 0 | 0 | 0.00% | 0 | 0 | 0.00% | 3 | 3 | 5.08% |
| | hyperaemia | | | | | | | | | | | | |
| | Eye irritation | 1 | 1 | 1.69% | 0 | 0 | 0.00% | 0 | 0 | 0.00% | 0 | 0 | 0.00% |
| | Eye secretion | 0 | 0 | 0.00% | 0 | 0 | 0.00% | 1 | 1 | 1.69% | 1 | 1 | 1.69% |
| | Eye pruritus | 0 | 0 | 0.00% | 1 | 1 | 1.72% | 2 | 2 | 3.39% | 2 | 2 | 3.39% |
| | Eye pain | 11 | 9 | 15.25% | 16 | 14 | 24.14% | 12 | 11 | 18.64% | 12 | 12 | 20.34% |
| | Foreign body | 5 | 5 | 8.47% | 2 | 2 | 3.45% | 4 | 3 | 5.08% | 2 | 2 | 3.39% |
| | sensation | | | | | | | | | | | | |
| | in eyes | | | | | | | | | | | | |
| | Meibomian | 0 | 0 | 0.00% | 0 | 0 | 0.00% | 1 | 1 | 1.69% | 2 | 2 | 3.39% |
| | gland | | | | | | | | | | | | |
| | dysfunction | | | | | | | | | | | | |
| | Hordeolum | 0 | 0 | 0.00% | 1 | 1 | 1.72% | 0 | 0 | 0.00% | 0 | 0 | 0.00% |

Note:
Incidence = (number of cases in each group/total number of people in each group) * 100%

Adverse events related to the study drug occurred in 3 system organ categories, namely infections and infestations, various examinations and eye disorders. The most frequent occurrences were successively eye pain, blurred vision, and foreign body sensation in eyes. The frequencies of each group (A, B, C and D) were calculated and compared, and there was no statistical difference between groups (p=0.7291, p>0.05).

TABLE 13
Occurrence of drug-related adverse events by system in the four groups (SS)

| | | Group A | | | Group B | | | Group C | | | Group D | | |
|---|---|---|---|---|---|---|---|---|---|---|---|---|---|
| | | | Number | | | Number | | | Number | | | Number | |
| | | No. of | of | Per- | No. of | of | Per- | No. of | of | Per- | No. of | of | Per- |
| SOC name | PT name | cases | subjects | centage | cases | subjects | centage | cases | subjects | centage | cases | subjects | centage |
| Infections | | 2 | 1 | 1.69% | 0 | 0 | 0.00% | 0 | 0 | 0.00% | 0 | 0 | 0.00% |
| and | | | | | | | | | | | | | |
| infestations | | | | | | | | | | | | | |
| | Nasopharyngitis | 2 | 1 | 1.69% | 0 | 0 | 0.00% | 0 | 0 | 0.00% | 0 | 0 | 0.00% |
| Investigations | | 0 | 0 | 0.00% | 0 | 0 | 0.00% | 1 | 1 | 1.69% | 1 | 1 | 1.69% |
| | Alanine | 0 | 0 | 0.00% | 0 | 0 | 0.00% | 0 | 0 | 0.00% | 1 | 1 | 1.69% |
| | aminotransferase | | | | | | | | | | | | |
| | increased | | | | | | | | | | | | |
| | Blood bilirubin | 0 | 0 | 0.00% | 0 | 0 | 0.00% | 1 | 1 | 1.69% | 0 | 0 | 0.00% |
| | increased | | | | | | | | | | | | |
| Eye | | 18 | 13 | 22.03% | 25 | 17 | 29.31 | 23 | 14 | 23.73% | 18 | 15 | 25.42% |
| disorders | | | | | | | | | | | | | |
| | Dry eye | 2 | 2 | 3.39% | 3 | 3 | 5.17% | 3 | 3 | 5.08% | 1 | 1 | 1.69% |
| | Keratopathy | 0 | 0 | 0.00% | 0 | 0 | 0.00% | 1 | 1 | 1.69% | 0 | 0 | 0.00% |
| | Conjunctival | 0 | 0 | 0.00% | 1 | 1 | 1.72% | 1 | 1 | 1.69% | 0 | 0 | 0.00% |
| | congestion | | | | | | | | | | | | |
| | Lacrimation | 1 | 1 | 1.69% | 1 | 1 | 1.72% | 0 | 0 | 0.00% | 0 | 0 | 0.00% |
| | increased | | | | | | | | | | | | |
| | Hypoaesthesia | 1 | 1 | 1.69% | 0 | 0 | 0.00% | 0 | 0 | 0.00% | 0 | 0 | 0.00% |
| | eye | | | | | | | | | | | | |
| | Blurred vision | 1 | 1 | 1.69% | 0 | 0 | 0.00% | 4 | 3 | 5.08% | 3 | 3 | 5.08% |
| | Photophobia | 1 | 1 | 1.69% | 2 | 2 | 3.45% | 0 | 0 | 0.00% | 0 | 0 | 0.00% |
| | Eye irritation | 1 | 1 | 1.69% | 0 | 0 | 0.00% | 0 | 0 | 0.00% | 0 | 0 | 0.00% |
| | Eye secretion | 0 | 0 | 0.00% | 0 | 0 | 0.00% | 1 | 1 | 1.69% | 1 | 1 | 1.69% |
| | Eye pruritus | 0 | 0 | 0.00% | 1 | 1 | 1.72% | 1 | 1 | 1.69% | 1 | 1 | 1.69% |
| | Eye pain | 9 | 8 | 13.56% | 15 | 13 | 22.41% | 8 | 8 | 13.56% | 11 | 11 | 18.64% |
| | Foreign body | 2 | 2 | 3.39% | 2 | 2 | 3.45% | 3 | 2 | 3.39% | 1 | 1 | 1.69% |
| | sensation in | | | | | | | | | | | | |
| | eyes | | | | | | | | | | | | |
| | Meibomian | 0 | 0 | 0.00% | 0 | 0 | 0.00% | 1 | 1 | 1.69% | 0 | 0 | 0.00% |
| | gland | | | | | | | | | | | | |
| | dysfunction | | | | | | | | | | | | |

Note:
Incidence = (number of cases in each group/total number of people in each group) * 100%

The results of this study showed that there were no statistical differences in drug-related adverse events, adverse events leading to dropout, serious adverse events, drug-related adverse events leading to dropout between test groups (A, B, C) and control group (D) (P >0.05). There were no significant differences in vital signs, physical examination, laboratory examination (blood routine examination, urine routine examination, blood biochemistry), 12-lead electrocardiogram and other safety evaluation indicators between test groups (A, B, C) and control group (D) (P >0.05). In this study, the most frequent adverse events related to the study drug were successively eye pain, blurred vision, and foreign body sensation in eyes, which were consistent with the adverse reactions recorded in the instructions of the control drug, and were expected adverse reactions. There was no significant difference between test groups (A, B, C) and control group (D) (P >0.05). No serious adverse events related to the study drug were found in this study. Among the test groups (A, B and C), group A had the lowest incidence of adverse events.

There was no statistical difference in the efficacy among groups A, B, C and D. However, groups A, B and C showed a better effect trend than group D in terms of primary efficacy indicators and most of the secondary efficacy indicators. In terms of safety, group A had the lowest incidence of adverse events. The frequency of administration was lower in group A compared with groups B and D at the same concentration, and the concentration was lower in group A compared with group C. Therefore, group A was selected as the phase III test group, and the sample size was further expanded to verify the effectiveness and safety of cyclosporin A ophthalmic gel in the treatment of patients with moderate to severe dry eyes.

### Example 7 Phase III Clinical Trial

This Phase III Clinical Trial has been disclosed in International Application No. PCT/CN2021/126462, and Peng W, Jiang X, Zhu L, et al. Cyclosporine A (0.05%) Ophthalmic Gel in the Treatment of Dry Eye Disease: A Multicenter, Randomized, Double-Masked, Phase III, COSMO Trial. Drug Des Devel Ther. 2022; 16:3183-3194. Published 2022 Sep. 20. doi:10.2147/DDDT.S370559. These two patent application and publication are incorporated by reference in their entireties.

### 1 Study Background

Dry eye disease (DED) is a common ophthalmic disease. It is caused by tear film instability and/or ocular surface damage due to abnormal tear quantity or quality or fluid dynamics, resulting in eye discomfort and visual dysfunction. It is mainly manifested as eye dryness, burning sensation, eye redness, photophobia, tears, foreign body sensation, eyelid itching, visual acuity reduced, etc. These symptoms have a significant impact on the patient's visual function, daily activities, social and physical functions.

In recent years, with the popularity of computers, the aging of the population, the use of contact lenses and other reasons, the incidence of dry eye disease is increasing. According to the existing epidemiological studies in China, the incidence of dry eye disease in China is similar to that in other Asian countries and higher than that in the United States and Europe, with an incidence of about 21%˜30/o. Dry eye disease has become one of the most common clinical diseases.

There is no uniform classification standard for dry eye disease in the world, and there are multiple classification methods. According to China's Expert Consensus on the Clinical Diagnosis and Treatment of Dry Eye (2013), dry eye disease can be divided into five categories: 1) aqueous tear deficiency dry eye, 2) evaporative dry eye, 3) mucin deficiency dry eye, 4) abnormal tear dynamics, and 5) mixed dry eye. Among them, mixed dry eye is the most common type of dry eye in clinic, which is caused by the above two or more reasons. The indication for this clinical study is moderate to severe dry eye.

The etiology of dry eye is complex, and inflammation plays a key role in the pathogenesis of dry eye, and such inflammation is a non-infectious immune-related inflammation. At present, the commonly used treatment is the topical application of artificial tears, which can improve the patient's ocular discomfort symptoms, but fails to treat the cause. It is clinically proven that anti-inflammatory treatment can not only effectively relieve the symptoms and signs of dry eye, but also slow the progression of the disease.

Cyclosporine A (CsA) is a selective and potent immunosuppressant. CsA plays a therapeutic role in dry eye by inhibiting the aggregation of inflammatory cells and the release of inflammatory factors, reducing T lymphocytes infiltration and inhibiting the apoptosis of ocular surface epithelial cells. The phase III clinical trial of CsA in the United States showed that cyclosporine A has broad prospects in the treatment of dry eye through anti-inflammatory and immunomotor effects.

At present, cyclosporine preparations that have been approved for marketing in China include cyclosporine eye drops (3 mL: 30 mg) produced by North China Pharmaceutical Co., Ltd., which are used to prevent and treat immune rejection response after corneal transplantation. After this study was carried out, the cyclosporine eye drops (II) (0.4 mL:0.2 mg) produced by Shenyang Xingqi Pharmaceutical Co., Ltd. was approved for marketing in June 2020, which is used to promote the tear secretion in patients with dry eye and for patients with reduced tear production due to ocular inflammation associated with keratoconjunctival sicca. The cyclosporine ophthalmic emulsion (Restasis®, containing 0.05% cyclosporin A) that has been marketed abroad for dry eye treatment was developed by Allergan and was approved by the US FDA in December 2002. It was launched in the US in the second quarter of 2003 and in Canada in 2010, with good sales. It has been reported in the literature that cyclosporine ophthalmic emulsion (Restasis®) also has a certain effect on meibomian gland dysfunction, and the onset of meibomian gland dysfunction is often associated with dry eye.

The drug used in the methods disclosed herein is cyclosporine A ophthalmic gel. In consideration of the medication safety and the effective increase of drug exposure, the dosage form of the drug used in the methods disclosed herein is gel. Human eyes have a special protective mechanism, ordinary eye drops will be quickly eliminated with low bioavailability. The pharmaceutical excipient carbomer can form a uniform, transparent and stable hydrogel at pH 6-12 with good adhesion, which is suitable for eye administration. Compared with ophthalmic emulsion, it can prolong the action time of drugs in the eye, and has no greasy and no irritation to mucous membrane.

Anhui Academy of Medical Sciences and Shanghai Ruizhi Chemical Research Co., Ltd. were commissioned to conduct a comparative study on eye irritation, pharmacokinetics and pharmacodynamics of this product compared with cyclosporine ophthalmic emulsion (Restasis®). The results showed that: 1) After four weeks of continuous administration to the eyes of rabbits, there was no irritation to the eyes of rabbits. 2) After the two study preparations were administered to New Zealand white rabbits, both of them had a large amount of exposure in tears, conjunctiva and cornea, and the highest in the cornea; while there was no exposure in plasma, lacrimal glands and anterior aqueous humor, indicating that after being administered on ocular surface, cyclosporin A can only be distributed on the surface tissues of the eyeball, and will not penetrate into deep tissues and systemic circulation. The exposure amounts of cyclosporin A ophthalmic gel in the cornea, conjunctiva and tear were 5.25, 2.55 and 1.67 times that of the cyclosporine ophthalmic emulsion (Restasis®), respectively. Pharmacodynamics test results showed that the two preparations significantly increased the tear secretion of New Zealand white rabbits, and there was no significant difference between them.

From December 2017 to November 2019, a multi-center, randomized, single-blind, controlled, dose-finding phase II clinical study was conducted on the efficacy and safety of cyclosporine A ophthalmic gel in the treatment of patients with moderate to severe tear deficiency dry eyes in 13 study sites including Zhongshan Ophthalmic Center, Sun Yat-sen University, involving 240 subjects. The results showed that EDS score (eye dryness score) of each dose group of cyclosporin A ophthalmic gel and cyclosporine ophthalmic emulsion (Restasis®) decreased from baseline after 12 weeks of treatment, with statistically significant differences within the groups, and no statistically significant differences between groups after baseline correction by covariance analysis. Other secondary efficacy indicators, such as Mean changes in VAS scores of the other 6 dry eye symptoms (burning sensation/stinging sensation, itching, foreign body sensation, discomfort, photophobia and pain) except for EDS from baseline and mean change of corneal fluorescein staining score from baseline, indicated that each dose group of cyclosporine A ophthalmic gel had a better effect trend than cyclosporine ophthalmic emulsion (Restasis®).

In terms of safety, there were no statistical differences in drug-related adverse events, adverse events leading to dropout, serious adverse events, drug-related adverse events leading to dropout between each dose group of cyclosporine A ophthalmic gel and cyclosporine ophthalmic emulsion (Restasis®) (p >0.05). There were no significant differences in vital signs, physical examination, laboratory examination (blood routine examination, urine routine examination, blood biochemistry), 12-lead electrocardiogram and other safety evaluation indicators between each dose group of cyclosporine A ophthalmic gel and cyclosporine ophthalmic emulsion (Restasis®) (p >0.05). In this Phase II study, the most frequent adverse events related to the study drug were successively eye pain, foreign body sensation in eyes, and blurred vision, which were consistent with the adverse reactions recorded in the instructions of the control drug, and were expected adverse reactions. There was no significant difference between each dose group of cyclosporine A ophthalmic gel and cyclosporine ophthalmic emulsion (Restasis®) (p >0.05). No serious adverse events related to the study drug were found during the study period.

According to China's Expert Consensus on the Clinical Diagnosis and Treatment of Dry Eye (2020) and the experience of cyclosporine ophthalmic emulsion (Restasis®) in dry eye clinical study abroad, the indication of cyclosporine A ophthalmic gel is moderate to severe dry eye (Kenneth, Sall, and, et al. Two multicenter, randomized studies of the efficacy and safety of cyclosporine ophthalmic emulsion in moderate to severe dry eye disease. Ophthalmology, 107(4): pp. 631-691 (2000)). To further validate the efficacy and safety of cyclosporine A ophthalmic gel in patients with moderate to severe dry eye, we conducted this Phase III, multi-center, randomized, double-blind, vehicle-controlled, optimal-effect design clinical study in China.

### 2 Study Objectives

The purpose of this phase III clinical study was to to evaluate the efficacy and safety of cyclosporine A ophthalmic gel compared with vehicle in patients with moderate to severe dry eye.

### 3 Trial Design and Implementation

### 3.1 Overall Design and Implementation of Trial

The clinical trial of cyclosporine A (CsA) ophthalmic gel (COSMO trial) was a multicenter, randomized, double-masked, vehicle-controlled phase III clinical trial. Patients with moderate-to-severe DED treated in the Department of Ophthalmology of 37 hospitals in China between November 2020 and April 2021 were enrolled. This study was registered with ClinicalTrials.gov (identifier, NCT04541888). The study protocol was approved by the ethics committees of all participating centers and adhered to the tenets of the Declaration of Helsinki. All participants provided written informed consent before enrollment.

This study was a phase III, multi-center, randomized, double-blind, vehicle-controlled, optimal-effect design clinical study.

The results of the phase 11 dose-finding study showed that, EDS score (eye dryness score) of each dose group of cyclosporin A ophthalmic gel [Group A (0.3 g: 0.15 mg, q.d.); Group B (0.3 g: 0.15 mg, b.i.d.); Group C (0.3 g: 0.3 mg, q.d.)] and cyclosporine ophthalmic emulsion (Restasis®, 0.4 mL: 0.2 mg, b.i.d.) decreased from baseline after 12 weeks of treatment, with statistically significant differences within the groups, and no statistically significant differences between groups after baseline correction by covariance analysis. Each dose group of cyclosporine A ophthalmic gel had a better effect trend than cyclosporine ophthalmic emulsion (Restasis®). Among them, group A (0.3 g: 0.15 mg, q.d.) had the lowest incidence of adverse events and adverse reactions, so cyclosporin A ophthalmic gel Group A (0.3 g: 0.15 mg, q.d.) was selected as the test group of phase III study.

Patients with a single eye disease, whose diseased eye met the inclusion criteria, could be enrolled for treatment, and the diseased eye was used as the eye under study. Patients with disease in both eyes received treatment for both eyes. If only one eye met the inclusion criteria, that eye was used as the eye under study. If both eyes met the inclusion criteria, the eye with higher ICSS score was used as the eye under study, and if the ICSS results were the same, the right eye was used as the eye under study.

The screening period was 21 days, and the treatment period was 84±6 days. After the successful screening, the subjects should be washed out for at least 14 days. The study included 5 visits: Visit 1 (D-21˜D-15, screening period), Visit 2 (D0, Baseline/randomization), Visit 3 (D14±2), Visit 4 (D42±4), Visit 5 (D84±6, end of treatment).

### 3.2 Selection of Control Drug

Before this study, there was no cyclosporine A preparation for the treatment of dry eye on the market in China. According to the “General Considerations to Clinical Trials for Drug”, vehicle control is generally recommended for control group selection.

With reference to clinical studies of cyclosporine preparation in dry eye abroad and relevant literature reports at home and abroad, the cyclosporin A preparations currently on the market worldwide for the treatment of dry eye include Restasis® from Allergan, Ikervis® from Santen Pharmaceutical Co. Ltd. and Cequa® from Sun Pharma, all of which were studied in vehicle-controlled phase III trials. (Kenneth, Sall, and, et al. Two multicenter, randomized studies of the efficacy and safety of cyclosporine ophthalmic emulsion in moderate to severe dry eye disease. Ophthalmology, 2000. 107(4): p. 631-691; Leonardi, A., E. M. Messmer, M. Labetoulle, et al. Efficacy and safety of 0.1% ciclosporin A cationic emulsion in dry eye disease: a pooled analysis of two double-masked, randomised, vehicle-controlled phase III clinical studies. British Journal of Ophthalmology, 2018; Kim, E. C., J.-S. Choi, C.-K. Joo. A Comparison of Vitamin A and Cyclosporine A 0.05% Eye Drops for Treatment of Dry Eye Syndrome. American Journal of Ophthalmology, 2009. 147(2): p. 0-213000; Lin Bijuan, Liu Zuguo, Wang Xiaoyun, et al. A comparative study of cyclosporine in the treatment of moderate to severe dry eye, Journal of Sun Yat-sen University (Medical Sciences), 2006. 27(3))

The concentration of carbomer eye drops approved in China for the treatment of dry eye is 0.2%. The concentration of the excipient carbomer in cyclosporine A ophthalmic gel is 0.06%, that is, the concentration of carbomer in vehicle is 0.06%, which can lubricate the eye surface.

Therefore, this clinical study adopts a vehicle-controlled, superior-effect design. The physical properties of the vehicle, such as appearance, size, color, dosage form, weight, taste and odor, are the same as those of the test drug.

### 3.3 Ways to Reduce Bias

In order to ensure the balance of the enrolled cases, as well as the scientificity, objectivity and reliability of the trial data, this clinical study will be carried out in several clinical study units across the country, using a randomized, double-blind design. The enrolled subjects will be randomly assigned to the cyclosporine A ophthalmic gel group (test group) and vehicle group (control group) in a 1:1 ratio. Adopt the method of block randomization and competitive entry.

The investigator will register relevant information of subjects who meet the inclusion criteria but not the exclusion criteria into the network random center, and receive the random number and drug number of the subject in the network random center according to the order of login time. The random number obtained by the investigator from the online random center is the unique number of the subject. The network random system guarantees the randomness of case selection. All personnel involved in this study should make every effort to ensure the strict implementation of the randomization scheme.

### 4 Subject Selection Criteria

### 4.1 Indications

Moderate to severe dry eyes.

### 4.2 Inclusion Criteria

1) Age from 18 to 80 years old, regardless of gender;
2) According to the subject's symptoms and signs and eye examination, the following 4 points must be met at the same time to be diagnosed as moderate to severe dry eye:
a) EDS score, eye dryness score (VAS evaluation of 0-100 points), ≥40 points;
b) Break-up time (BUT) <10 s;
c) Schirmer test result <10 mm/5 min;
d) Corneal fluorescein staining score (ICSS) ≥2 points;
3) Agreed to participate in the study and voluntarily signed the informed consent form.

### 4.3 Exclusion Criteria:

1) Patients with severe dry eye who need surgical treatment;
2) Patients who underwent internal eye surgery within 12 months before screening or who needed internal eye surgery during the study period; patients who underwent eyelid surgery within 6 months before screening;
3) Patients treated with permanent lacrimal point blocking or those treated with temporary lacrimal point blocking within 6 months before screening;
4) Dry eye caused by surgery;
5) Patients with glaucoma;
6) Patients who cannot avoid wearing contact lenses during the trial;
7) Active infection of the whole body or eyes;
8) Patients with multiple episodes of viral keratitis:
9) Patients with ocular cicatricial pemphigoid, obvious conjunctival scars, ocular chemical burns, and neurotrophic keratoconjunctivitis;
10) Patients suffering from malignant tumors in the past 5 years, except for basal cell carcinoma of skin, squamous cell carcinoma in situ of skin, and cervical cancer in situ that have been completely cured;
11) Perimenopausal women who are using hormone replacement therapy;
12) Patients who cannot stop using other ophthalmic preparations such as eye drops after the elution drug is distributed;
13) Patients with severe cardiopulmonary diseases, uncontrollable hypertension and diabetes that affect the collection of study parameters or compliance;
14) According to laboratory tests, alanine aminotransferase (ALT) and aspartate aminotransferase (AST) ≥2 times the upper limit of normal, and serum creatinine (Cr) ≥1.5 times the upper limit of normal;
15) Patients who have a serious nervous system disorder or which the investigator considers likely to affect the subjects' participation in the study or follow-up or to result in the subjects' hospitalization during the study;
16) Women who were pregnant or lactating, or women or men of childbearing age who cannot take effective contraceptive methods during the study period;
17) Those who have participated in or are currently participating in other clinical trials within 1 month before screening;
18) Those who used cyclosporine systemically or locally within one month before screening;
19) Patients who are known to be allergic to cyclosporine, fluorescein or any component of the study drug;
20) Patients considered by the investigator to be unsuitable to participate in the study, including those who are unable or unwilling to comply with the requirements of the protocol.
   4.4 Subject's Withdrawal from the Study
   4.4.1 Subject's Withdrawal from the Study Determined by the Investigator

The investigator reserves the right to exclude the subjects from the trial if the investigator considers that continuing the trial might affect the safety of the subjects, or if the subjects fail to comply with the requirements for participating in the study or refuse to perform the trial procedures.

4.4.2 Subjects Withdraw from the Trial on their Own

Subjects are unwilling to continue in clinical trials. In accordance with the Declaration of Helsinki and informed consent form, subjects have the right to withdraw from the study at any stage without affecting their subsequent treatment.

### 4.4.3 Treatment of Subject Withdrawal

For subjects who withdraw from the study, the investigator should try to understand the reasons for their withdrawal. If possible, the subjects should complete the visit 5 and related examinations at study site within 7 days after the last medication, and the evaluation results should be recorded in detail. If an adverse event occurs, the outcome of the adverse event should be tracked (see Example 7 10.3 AE records for details).

### 4.5 Early Termination or Suspension of the Study

The study may be terminated or suspended early. The sponsor reserves the right to stop the research and development of drugs at any time. The party that decides to suspend/terminate the study will send a written notice and the reasons for the termination or suspension of the study to the investigator, sponsor, and regulatory agency.

Reasons for early termination or suspension of the study may include:
1) An identified risk to the subject that is unexpected, significant, or unacceptable;
2) Existing efficacy results support early termination of the study;

The study may continue once the above-mentioned drug safety, protocol compliance, and data quality issues causing the study suspension are resolved and the sponsor, ethics committee or regulatory authority agrees.

### 5 Study Drugs Medication Regimens and Concomitant Treatments

### 5.1 Study Drugs

### 5.1.1 Test Drug

| | |
|---|---|
| Drug name | Cyclosporine A Ophthalmic Gel |
| Strength | 0.3 g:0.15 mg |
| Expiration date | Two years |
| Storage condition | 15-25° C. |
| Batch No. | |
| Supplier | Zhaoke (Guangzhou) Ophthalmology Pharmaceutical |
| | Limited |
| Manufacturer | Zhaoke (Guangzhou) Ophthalmology Pharmaceutical |
| | Limited |
| Adverse reactions | Phase II clinical study results showed that the most |
| | common adverse reactions were eye pain (18.18%), |
| | foreign body sensation in eyes (3.98%), and blurred |
| | vision (2.84%). |

### 5.1.2 Control Drug

| | |
|---|---|
| Drug name | Vehicle |
| Strength | 0.3 g:0 mg |
| Expiration date | Two years |
| Storage condition | 15-25°C |
| Batch No. | |
| Supplier | Zhaoke (Guangzhou) Ophthalmology Pharmaceutical |
| | Limited |
| Manufacturer | Zhaoke (Guangzhou) Ophthalmology Pharmaceutical |
| | Limited |

### 5.1.3 Basic Drug

| | |
|---|---|
| Drug name | Hypromellose eye drops |
| Strength | 10 ml:50 mg |
| Expiration date | Two years |
| Storage condition | Airtight preservation |
| Batch No. | |
| Supplier | Zhaoke (Guangzhou) Ophthalmology Pharmaceutical |
| | Limited |
| Manufacturer | Shanghai Sine Jinzhu Pharmacy Co., Ltd. |
| Adverse reactions | In rare cases, it may cause eye discomfort, such as eye |
| | pain, blurred vision, persistent conjunctival congestion |
| | or eye tingling. |

### 5.2 Packaging, Labeling and Preparation of Study Drugs

### 5.2.1 Randomization Method

In this study, the method of block randomization and competitive entry were adopted for each site. Randomized tables were generated using SAS statistical software by an unblinded statistician independent of the project, and subjects were assigned to test drugs or control drugs in a 1:1 ratio. After the screening of each qualified subject by the researchers of all sites participating in the study, the investigator logged into the randomization system using the account number, entered the relevant information of the subject, and obtained the randomization number and corresponding drug number of the subject. The random number of the subject was composed of three digits, and the drug number was composed of English letter “D” plus three digits. The investigator dispensed study drugs according to drug numbers. The principal investigator of each study site had their own login accounts and passwords to ensure the security and integrity of the login system.

### 5.2.2 Blind, Packaging and Labeling

The drug packaging of the test group and the control group was uniformly provided by Zhaoke (Guangzhou) Ophthalmology Pharmaceutical Limited, and was packaged by non-participants in the clinical study according to the random allocation table. Each carton contains enough drugs for the subject during the washout period and the treatment period. During the packaging process, the packaging records shall be filled in strictly according to the requirements, and be signed by the relevant responsible person for confirm them. Paste the label “For clinical study only” on the outer packaging box.

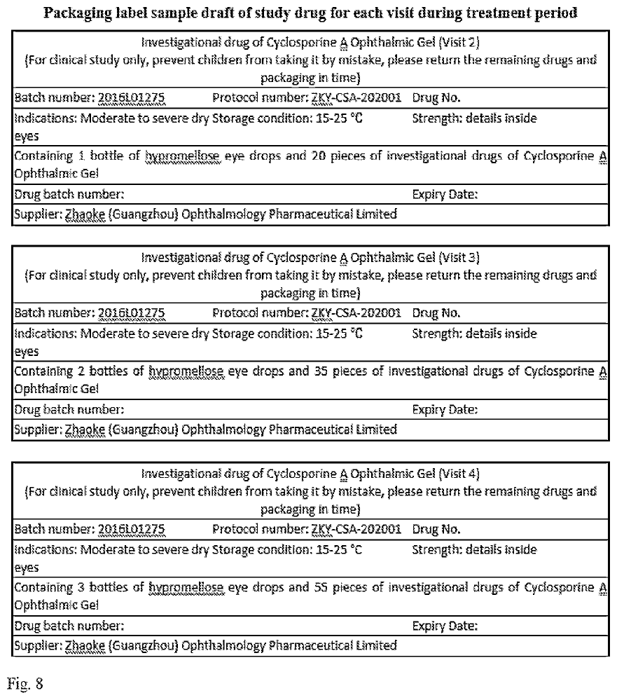

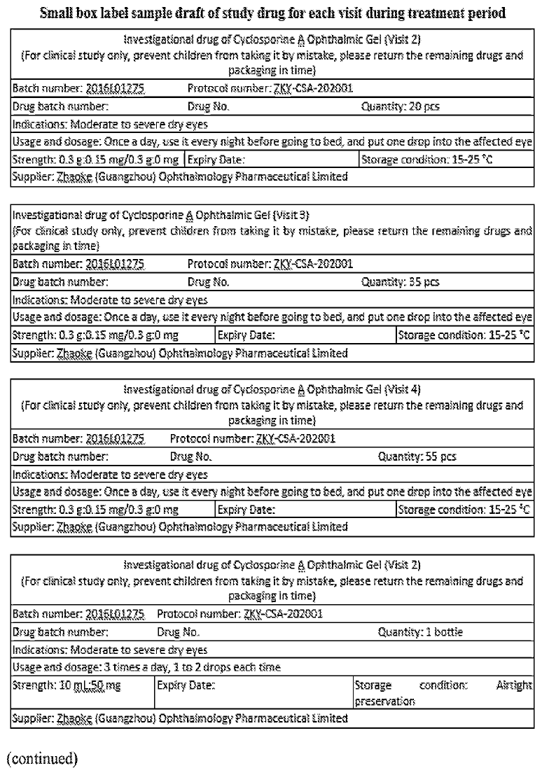

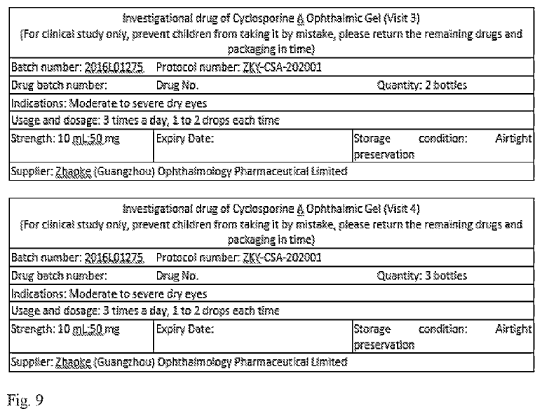

The packaging and labeling of study drugs must comply with current drug instructions and label management regulations. The label shall include at least the following information: study drug number, strength, date of manufacture or expiration, batch number, name of the sponsor, and storage conditions. The label sample draft is shown in FIGS. 7-9.

### 5.3 Emergent Unblinding

During the course of the study, when the subject has an emergency or critical condition that requires medical treatment and the investigator considers it necessary to identify the subject's medication, it is necessary to know the study drugs the subject is receiving for further treatment. Emergency unblinding can be performed by logging into the random system.

### 5.4 Drug Management

### 5.4.1 Drug Reception

Study drugs distributed to each study site by the drug distribution center entrusted by the sponsor shall be received by the designated person of each study site, and the receipt record shall be signed.

### 5.4.2 Drug Registration

The study drugs in each study site are managed by dedicated personnel:
Record the inventory of all study drugs;
All study drugs are only distributed and used in accordance with the provisions of the study protocol;
Record the date and quantity of drug distribution and recovery for each subject;
Distribute study drugs with corresponding drug numbers to subjects according to the drug numbers obtained;
Confirm that all unused study drugs and outer packaging are returned to the sponsor and make records;
Make an inventory of study drugs throughout the study and provide an inventory record;
The quantity of drugs in stock should be consistent with the quantity of drugs received, distributed and expired, and any deviation must be explained in the remarks.
The quantity of drugs distributed must be consistent with the quantity of drugs used and recovered, and any deviation must be explained in the remarks.

### 5.4.3 Drug Distribution and Recovery

In each study unit, the investigator will log in to the online random system to obtain the subject's random number and drug number in accordance with the order in which they are treated. The random number and drug number will remain unchanged during the study process. According to the drug number of each subject, the drug management personnel will distribute the study drug with the corresponding drug number to the subject. The investigator instructs the subjects to return the remaining drugs and drug packaging to the drug management personnel at the next follow-up visit. The drug management personnel timely and accurately fill in the drug distribution and recovery record form.

### 5.4.4 Drug Preservation

1) Keep drugs in accordance with the requirements of drug storage conditions, and drug management personnel should regularly monitor the temperature;
2) The special storage cabinets for experimental drugs are only open to the personnel responsible for drug management authorized by the investigator, and no study drugs are provided to anyone other than the subjects in the study.

### 5.4.5 Remaining Drugs

Due to the subjects' failure to take medication on time, withdrawal during the course of treatment or change of treatment regimen, any drugs left will be recalled and recorded in detail. The remaining drugs will be returned to the sponsor at the end of the study and destroyed uniformly.

### 5.5 Medication Regimens

After signing the informed consent form, subjects who meet the inclusion criteria but not the exclusion criteria will be enrolled in the study after screening.

At Visit 1, the qualified subjects are given the basic drug and the vehicle for washout. After the successful screening, the subjects should be washed out for at least 14 days.
Basic drug: 3 times a day, 1 to −2 drops each time.
Vehicle: once a day, use it every night before going to bed, and put one drop into the affected eye (the interval between it and basic drug should be more than 5 minutes).

At Visit 2, the investigator will log in to the online random system to obtain the subject's random number and drug number, which will remain unchanged during the study process. Subjects will be randomly assigned to the test group or control group. According to the drug number of each subject, the drug management personnel will distribute the study drug with the corresponding drug number to the subject. At Visit 2, 3 and 4, the basic drugs and study drugs will be distributed. Usage and dosage of basic drugs and study drugs:
Basic drug: Start on D0, 3 times a day, 1 to −2 drops each time.
Study drug: Start on D0, once a day, use it every night before going to bed, and put one drop into the affected eye (the interval between it and basic drug should be more than 5 minutes).

### 5.6 Concomitant Treatments

### 5.6.1 Concomitant Drugs

All concomitant drugs taken by the subject after signing the informed consent shall be recorded with their trade name or generic name. The trade name is used for drug consisting of multiple ingredients. The date of its start and end of use shall be recorded. If the exact start date is unknown, the investigator should indicate the year the drug was started if possible.

In case of symptomatic eye infection during the study, anti-infection treatment should be carried out according to the adverse event management procedures and clinical experience.

Antirheumatic analgesics, cannabinoid analgesics, opioid analgesics, anesthetics, antihistamines, antidepressants, antibronchodilators, antiarrhythmics, anti-parkinsonian drugs, antipsychotics drugs, anticonvulsants, anti-congestive drugs, adrenergic receptor blockers, thiazide diuretics, antileprotics, antimalarials, antineoplastic drugs, anxiolytics/hypnotics, herbs and vitamins, calcium regulators, estrogen Hormone replacement/anti-androgens, neurotoxins, sedatives and other immunomodulators. The above systemic drugs are known or suspected to cause, promote or aggravate dry eye, and should be avoided. If the investigator considers it necessary based on the patient's condition, it should be recorded in detail.

The following medications are prohibited during the study period:
1) Topical medications that may interfere with the effect of the study drug or the results of the study:
Antiglaucoma drugs, antiallergic drugs, antiviral drugs, anti-congestive drugs, miotics, mydriatics/cycloplegia, local anesthetics (except anesthetics in Schirmer test table), ocular non-steroidal anti-inflammatory drugs.
2) In addition to the investigational drugs of cyclosporine A ophthalmic gel and hypromellose eye drops, other topical ophthalmic drugs for the treatment of dry eye:
Artificial tears: sodium hyaluronate eye drops, polyvinyl alcohol eye drops, etc.; lubricating ointment (eye ointment, gel): carbomer eye gel, etc.;
Local anti-inflammatory and immunosuppressive agents: cyclosporine A emulsion, tacrolimus, glucocorticoids, non-steroidal anti-inflammatory drugs, etc.;
Autologous serum;
Others: Beifushu (containing recombinant human fibroblast growth factor), vitamin A palmitate, etc.

### 5.6.2 Combined Dry Eye Physical Therapy

During the study period, it is forbidden to use any dry eye physical therapy, such as eye massage, hot compress, fumigation, meibomian gland heat pulse therapy, wet chamber lenses, etc.

### 5.7 Subject Medication Compliance

Compliance of test drugs: At visits 3, 4 and 5, the investigator asked the subjects whether they used drugs correctly every day and recorded the number of actual medications. Drug compliance was evaluated by calculating the proportion of the number of actual medications.

The formula is as follows:

Compliance (%)=the number of actual medications/the number of scheduled medications (if the subject makes visit within the visit window, the number of scheduled medications will be the number of medications that should have been used up to the day before the visit; if the subject makes visit in advance, the number of scheduled medications will be the number of medications that should be used up to the previous day of the lower limit of the visit window; if the subject makes visit outside the visit window, the number of scheduled medications will be the number of medications that should be used up to the previous day of the upper limit of the visit window)×100%. If the calculated value is within 80%-120/o, it is defined as good compliance, and if it is less than 80% or greater than 120%, it is poor compliance.

### 6 Study Procedures

Five visits will be conducted in this study. Clinical examination procedures: According to the principle of eye surface stimulation from small to large, all patients undergo ophthalmic examination in the following order: VAS score for dry eye symptoms, slit lamp microscopy, break-up time (BUT), corneal and conjunctival staining and photography, and Schirmer test (surface anesthesia). The above-mentioned examinations should be carried out in strict accordance with the order of study protocol formulation. The test papers for corneal and conjunctival staining and Schirmer used in the examination shall be provided by the sponsor. Please refer to the appendix of this study protocol for the operation standard SOP of VAS score for dry eye symptoms, slit lamp microscopy, break-up time (BUT), corneal and conjunctival staining and photography, and Schirmer test (surface anesthesia).

### 6.1 Visit 1 (Screening Period)

The visit time was from D-21 to D-15.

Before conducting any study-related procedures, the investigator must obtain the informed consent subjects and sign the informed consent form. The investigator selected suitable subjects based on the patient's medical history and examination results.

After obtaining the written informed consent form, perform the following steps:
1) Collect demographic data of subjects, including date of birth, gender, height (cm) and weight (kg);
2) Perform physical examination (including skin, lymph nodes, head and neck, chest, abdomen, spine and limbs, nervous system and others) and measure vital signs;
3) Collect the main complaints, the course of the disease, and ask about the symptoms of dry eye, including dryness, foreign body sensation, burning sensation, fatigue, discomfort, blurred vision, etc.;
4) Past medical history and past medications, especially the history of dry eye and the use of drugs for dry eye, as well as the current comorbidities and concomitant treatments;
5) Visual examination (with naked eyes);
6) Perform VAS score (including dryness, burning sensation/stinging sensation, itching, foreign body sensation, discomfort, photophobia and pain);
7) Perform slit lamp microscopy;
8) Perform break-up time (BUT) determination;
9) Perform corneal staining and photography, ICSS scoring;
10) Schirmer test;
11) Laboratory tests, including blood routine, blood biochemistry and urine routine;
12) Pregnancy test for women of childbearing age;
13) 12 lead ECG;
14) Distribute the eluting drugs (1 bottle of hypromellose eye drops, 25 pieces of investigational drugs of Cyclosporine A Ophthalmic Gel) and subject diary cards of visit 1, to guide subjects on medication methods;
15) Make an appointment for the next treatment date.

### 6.2 Visit 2 (Baseline/Randomization)

The visit time was D0.
1) Verify item by item that the subjects meet the inclusion criteria and do not meet the exclusion criteria;
2) Visual examination (with naked eyes);
3) Perform VAS score (including dryness, burning sensation/stinging sensation, itching, foreign body sensation, discomfort, photophobia and pain);
4) Perform slit lamp microscopy;
5) Perform break-up time (BUT) determination;
6) Perform corneal and conjunctival staining and photography, ICSS scoring, upload qualified photos to the cloud platform for central reading, and perform Oxford grading evaluation;
7) Schirmer test;
8) Measure vital signs;
9) 12 lead ECG;
10) Ask about the symptoms of dry eye, new comorbidities and changes in concomitant treatments;
11) Ask subjects for adverse events, and record the adverse events observed and reported by the subjects;
12) Check the medicines and packaging returned by the subjects, retrieve the subject diary cards, and ask the subjects whether to take the medicine as prescribed by the doctor every day;
13) For qualified subjects, the investigator will log in to the online random system to obtain the subject's random number and corresponding drug number in accordance with the order in which they are treated. The subject will receive the study drug with the drug number (1 bottle of hypromellose eye drops and 20 pieces of investigational drugs of Cyclosporine A Ophthalmic Gel);
14) Distribute the subject diary cards of Visit 2 to guide the subjects on the medication method, and instruct the subjects to continuously use the study drug every night from the same day;
15) Ask the subjects to return the remaining medicines and empty packages at the next visit;
16) Make an appointment for the next treatment date.

### 6.3 Visit 3

The visit time was study D14, and the time window was +2 days.
1) Perform VAS score (including dryness, burning sensation/stinging sensation, itching, foreign body sensation, discomfort, photophobia and pain);
2) Perform slit lamp microscopy;
3) Perform break-up time (BUT) determination;
4) Perform corneal and conjunctival staining and photography, ICSS scoring, upload qualified photos to the cloud platform for central reading, and perform Oxford grading evaluation;
5) Schirmer test;
6) Measure vital signs;
7) Ask about the symptoms of dry eye, new comorbidities and changes in concomitant treatments;
8) Ask subjects for adverse events, and record the adverse events observed and reported by the subjects;
9) Check the medicines and packaging returned by the subjects, retrieve the subject diary cards, ask the subjects whether to take the medicine as prescribed by the doctor every day, and evaluate the medication compliance;
10) Distribute the therapeutic drugs (2 bottles of hypromellose eye drops, 35 pieces of investigational drugs of Cyclosporine A Ophthalmic Gel) and subject diary cards of visit 3, to instruct subjects to return any remaining medications and empty packages at their next visit;
11) Make an appointment for the next treatment date.

### 6.4 Visit 4

The visit time was study D42, and the time window was +4 days.
1) Perform VAS score (including dryness, burning sensation/stinging sensation, itching, foreign body sensation, discomfort, photophobia and pain);
2) Perform slit lamp microscopy;
3) Perform break-up time (BUT) determination;
4) Perform corneal and conjunctival staining and photography, ICSS scoring, upload qualified photos to the cloud platform for central reading, and perform Oxford grading evaluation;
5) Schirmer test;
6) Measure vital signs;
7) Ask about the symptoms of dry eye, new comorbidities and changes in concomitant treatments;
8) Ask subjects for adverse events, and record the adverse events observed and reported by the subjects;
9) Check the medicines and packaging returned by the subjects, retrieve the subject diary cards, ask the subjects whether to take the medicine as prescribed by the doctor every day, and evaluate the medication compliance;
10) Distribute the therapeutic drugs (3 bottles of hypromellose eye drops, 55 pieces of investigational drugs of Cyclosporine A Ophthalmic Gel) and subject diary cards of visit 4, to instruct subjects to return any remaining medications and empty packages at their next visit;
11) Make an appointment for the next treatment date.

### 6.5 Visit 5

The visit time was study D84, and the time window was ±6 days.
1) Visual examination (with naked eyes);
2) Perform VAS score (including dryness, burning sensation/stinging sensation, itching, foreign body sensation, discomfort, photophobia and pain);
3) Perform slit lamp microscopy;
4) Perform break-up time (BUT) determination;
5) Perform corneal and conjunctival staining and photography, ICSS scoring, upload qualified photos to the cloud platform for central reading, and perform Oxford grading evaluation;
6) Schirmer test;
7) Perform physical examination (including skin, lymph nodes, head and neck, chest, abdomen, spine and limbs, nervous system and others) and measure vital signs;
8) Laboratory tests, including blood routine, blood biochemistry and urine routine;
9) Pregnancy test for women of childbearing age;
10) 12 lead ECG;
11) Ask about the symptoms of dry eye, new comorbidities and changes in concomitant treatments;
12) Ask subjects for adverse events, record the adverse events observed and reported by the subjects, and assess the outcome of all adverse events;
13) Check the medicines and packaging returned by the subjects, retrieve the subject diary cards, ask the subjects whether to take the medicine as prescribed by the doctor every day, and evaluate the medication compliance.

### 7 Observation Indicators

### 7.1 Medical History and Clinical Symptoms

At Visit 1, the investigator asked the patient in detail about the history of systemic and ocular diseases, the history of surgery, the history of systemic and ocular medications, and the wearing of contact lenses.

During Visits 1 to 5, the investigator asked the patients in detail about the symptoms of dry eye, including dryness, foreign body sensation, burning sensation, fatigue, discomfort, blurred vision, etc.

### 7.2 VAS Score

From Visit 1 to Visit 5, each subject drew his or her own discomfort in the corresponding item on the VAS score scale. The investigator then measured the length with ruler, with 0-100 mm corresponding to 0-100 points, and calculated the score: 0 point represents no discomfort, 100 points represents the greatest discomfort, keep the integer, and the higher the value, the more severe the symptoms. This score scale includes seven evaluation items including dryness, burning sensation/stinging sensation, itching, foreign body sensation, discomfort, photophobia and pain^{[12,13]}, among which EDS score refers to VAS score of dryness.

### 7.3 Slit Lamp Microscopy

From Visit 1 to Visit 5, slit lamp microscopy was performed in accordance with the procedures provided in the Appendix to observe the subject's eyelids, lid margins, meibomian glands, conjunctiva and cornea.

### 7.4 Break-Up Time (BUT) Determination

From Visit 1 to Visit 5, the break-up time (BUT) was measured in accordance with the procedures provided in the Appendix to evaluate the stability and quality of the tear film. Measure 3 times with an electronic stopwatch and take the mean value.

### 7.5 Corneal and Conjunctival Staining and Photography

From Visit 1 to Visit 5, corneal and conjunctival staining and photography were performed in accordance with the procedures provided in the Appendix. Only cornea fluorescein staining was performed at Visit 1, and cornea fluorescein staining and conjunctiva lissamine green staining were performed respectively from visits 2 to 5. After corneal fluorescein staining, the fixed ICSS evaluator at each site observed the cornea under slit lamp microscope and gave ICSS score. After completing the corneal conjunctiva staining and taking photos, the investigator uploaded the qualified photos to the cloud platform for central reading, and Oxford grading was obtained.

There are ICSS and Oxford grading methods for scoring corneal conjunctival staining. ICSS score is used to evaluate the presence and degree of damage of corneal epithelial cells on the ocular surface of subjects, as shown in the Appendix. Oxford grading is used to evaluate whether the improvement of subjects' ocular surface is clinically significant, as shown in the Appendix. (Holland, E. J., J. Luchs, P. M. Karpecki, et al. Lifitegrast for the Treatment of Dry Eye Disease: Results of a Phase III, Randomized, Double-Masked, Vehicle-Controlled Trial (OPUS-3). Ophthalmology, 2017. 124(1): p. 53-60; Tauber, J., P. Karpecki, R. Latkany, et al. Lifitegrast Ophthalmic Solution 5.0% versus Vehicle for Treatment of Dry Eye Disease: Results of the Randomized Phase III OPUS-2 Study. Ophthalmology, 2015. 122(12): p. 2423-31; Bron, A. J., V. E. Evans, J. A. Smith. Grading Of Corneal and Conjunctival Staining in the Context of Other Dry Eye Tests. Cornea, 2003. 22(7): p. 640-650)

### 7.6 Basic Tear Secretion Test—Schirmer Test (Surface Anesthesia)

From Visit 1 to Visit 5, perform a basic tear secretion test in accordance with the procedures provided in the Appendix to measure the subject's basic tear secretion.

### 7.7 Visual Examination

At Visit 1, 2 and 5, measure the uncorrected visual acuity. Record the results of the visual acuity examination in accordance with the decimal notation method. Normal visual acuity is 1.0. If even the largest optotypes (line 0.1) cannot be recognized at 5 m, the patient is instructed to gradually approach the visual acuity chart until the optotypes are recognized. Then, calculate according to the formula of V=d/D. If the 50 m (0.1 line) optotypes can be seen clearly at 3 m, the actual vision should be V=3 m/50 m=0.06. If the subject cannot identify the largest optotypes at 1 m from the visual acuity chart, please check the index. Check at 1 m from the chart and gradually moves closer until it can be correctly identified, and record the distance, such as “index, 30 cm”. If the index is still not recognized at 5 cm, check the manual operation and record the distance, such as “manual, 30 cm”. If the manual operation cannot be recognized, please check the light perception. Illuminate the test eye with a flashlight in a dark room. The other eye must be tightly covered to prevent light transmission. Test whether the patient can perceive light before their eyes, record the light perception or no light perception, and record the distance to see the light, generally up to 5 m.

### 7.8 Physical Examination and Vital Signs

At Visits 1 and 5, perform physical examination (including skin, lymph nodes, head and neck, chest, abdomen, spine and limbs, nervous system and others);

From Visit 1 to Visit 5, measure vital signs (body temperature, breathing, pulse and blood pressure).

### 7.9 Laboratory Test

At Visits 1 and 5, collect blood and urine samples of the subjects for routine blood, blood biochemistry, and urine routine tests. Laboratory tests are carried out in the laboratories of each study site, and any results beyond the normal range should be judged by the investigator whether it has clinical significance.
Blood routine: white blood cell (WBC) and white blood cell classification (eosinophil percentage, basophil percentage, neutrophil percentage, lymphocyte percentage, monocyte percentage), red blood cell (RBC), hemoglobin (Hb), platelets (PLT);
Blood biochemistry: aspartate aminotransferase (AST), alanine aminotransferase (ALT), urea/urea nitrogen (UREA/BUN), creatinine (Cr), total bilirubin (TBil), direct bilirubin (DBil), uric acid (UA), fasting blood glucose (GLU);
Urine routine: urine white blood cell (WBC), urine red blood cell (RBC), urine protein, urine glucose.

### 7.10 Pregnancy Test and 12 Lead ECG

At Visits 1, 2 and 5, perform a 12-lead ECG. At Visits 1 and 5, perform a blood/urine pregnancy test.

### 8 Efficacy Evaluation

### 8.1 Primary Efficacy Indicator

The proportion of patients whose corneal fluorescein staining score (ICSS) dropped by ≥1 point from baseline at Visit 5 (test eye).

### 8.2 Secondary Efficacy Indicators

1) Mean changes in EDS scores from baseline at visits 3, 4 and 5 (both eyes);
2) Mean changes in VAS scores of the other 6 dry eye symptoms (burning sensation/stinging sensation, itching, foreign body sensation, discomfort, photophobia and pain) except for EDS from baseline at visits 3, 4 and 5 (both eyes);
3) Mean change of corneal fluorescein staining score (ICSS) from baseline at visits 3, 4 and 5 (test eye);
4) Mean change of corneal staining score (Oxford grading) from baseline at visits 3, 4 and 5 (test eye);
5) Mean change of break-up time (BUT) from baseline at visits 3, 4 and 5 (test eye);
6) Changes of Schirmer test value from baseline at visits 3, 4, and 5 (test eye).

### 9 Safety Evaluation

### 9.1 Physical Examination and Vital Signs

At Visits 1 and 5, perform physical examination (including skin, lymph nodes, head and neck, chest, abdomen, spine and limbs, nervous system and others): From Visit 1 to Visit 5, measure vital signs (body temperature, breathing, pulse and blood pressure).

### 9.2 Laboratory Test and ECG

At Visits 1 and 5, collect blood and urine samples of the subjects for routine blood, blood biochemistry, and urine routine tests, and perform 12 lead ECG test.

### 9.3 Visual Acuity and Slit Lamp Microscopy

At Visit 1, 2 and 5, measure the uncorrected visual acuity.

From Visit 1 to Visit 5, slit lamp microscopy was performed in accordance with the procedure provided in the Appendix to observe the subject's eyelids, lid margins, meibomian glands, conjunctiva and cornea.

### 10 Adverse Events

### 10.1 Definition

### 10.1.1 Adverse Events (AEs)

An “adverse event” (AE) refers to an adverse medical event that occurs after a patient or clinical trial subject receives a drug, but is not necessarily related to the treatment. Therefore, an adverse event can be any adverse and unanticipated sign (including abnormal laboratory findings), symptom or illness related to use of the (study) drug in time, regardless of whether it is related to the drug. Any adverse events, whether serious or not, and whether related to therapeutic drugs or no, must be recorded on the electronic case report form.

### 10.1.2 Serious Adverse Event (SAE)

An adverse event is considered to be a serious adverse event when it meets any one or more of the following criteria:
1) Resulting in hospitalization;
2) Resulting in prolonged hospital stays;
3) Causing disability;
4) Affecting work ability;
5) Life-threatening and death;
6) Causing congenital malformations;
7) Other important medical events: These adverse events may not immediately threaten life or lead to death, but may endanger patients or result in the need for intervention measures to prevent the occurrence of the above outcome, which shall be determined based on medical scientific judgment.

### 10.1.3 Suspicious and Unexpected Serious Adverse Reactions (SUSAR)

It refers to adverse events that meet relevance, severity, and unexpectedness at the same time. The unexpectedness, for the test drug, means that the event is not listed in the investigator's manual, or its nature and severity are inconsistent with those described in the investigator's manual.

### 10.2 AE Assessment

### 10.2.1 Severity Determination

The severity or intensity of adverse events is classified into the following three levels:
Mild: easily tolerated and occasionally felt by the subject;
Moderate: So uncomfortable that it interferes with daily activities. Subjects feel obvious but tolerable symptoms and do not need to stop medication.
Severe: It interferes with normal daily activities, and the subjects feel that the symptoms are significant and intolerable, so they need to stop taking the drug.
   10.2.2 Relevance Evaluation with Test Drug

The causality judgment of adverse events in clinical trials shall be made by the authorized investigator. In addition to the judgment of whether there is a causal relationship with the test drug, the basis for the judgment should be explained as much as possible. The relevance evaluation standard is based on the five-level classification method of “definitely related, probably related, possibly related, possibly unrelated, and definitely unrelated”. (See Table 14)

TABLE 14
Study drug relevance criteria

| 5-level | |
|---|---|
| classification | Judgment criteria |
| Definitely | There is a reasonable relationship between the time of drug initiation and the occurrence of |
| related | adverse event; the adverse event is in line with the known adverse reaction types of the test |
| | drug; the occurrence of adverse event cannot be explained by factors other than the |
| | investigational drug (such as combined basic drug); the adverse event is relieved or |
| | disappeared after the investigational drug is reduced or stopped; a similar adverse event will |
| | occur when the investigational drug is used again (non-essential conditions). |
| Probably | There is a reasonable relationship between the time of drug initiation and the occurrence of |
| related | adverse event; the adverse event is in line with the known adverse reaction types of the test |
| | drug; the occurrence of adverse event can/cannot be explained by factors other than the |
| | investigational drug (such as combined basic drug); the adverse event can/cannot be relieved |
| | or disappeared after the investigational drug is reduced or stopped. |
| Possibly | There is a reasonable relationship between the time of drug initiation and the occurrence of |
| related | adverse event; the adverse event is in line with the known adverse reaction types of the test |
| | drug; the occurrence of adverse event can/cannot be explained by factors other than the |
| | investigational drug (such as combined basic drug); the adverse event can/cannot be relieved |
| | or disappeared after the investigational drug is reduced or stopped; it is not clear whether a |
| | similar adverse event will occur when the investigational drug is used again. |
| Possibly | There is a reasonable relationship between the time of drug initiation and the occurrence of |
| unrelated | adverse event; the adverse event is in line with the known adverse reaction types of the test |
| | drug; the occurrence of adverse event can/cannot be explained by factors other than the |
| | investigational drug (such as combined basic drug); the adverse event may be/will not be |
| | relieved or disappeared after the investigational drug is reduced or stopped; it is not clear |
| | whether a similar adverse event will occur when the investigational drug is used again. |
| Definitely | There is no reasonable relationship between the time of drug initiation and the occurrence of |
| unrelated | adverse event; the adverse event is not in line with the known adverse reaction types of the test |
| | drug; the occurrence of adverse event can be explained by factors other than the investigational |
| | drug (such as combined basic drug); the adverse event cannot be relieved or disappeared after |
| | the investigational drug is reduced or stopped; no similar adverse event occurs when the test |
| | drug is used again. |

### 10.3 AESAE Records

In this study, adverse events are collected from the first administration until 10 days after the last administration. AE/SAE records include but are not limited to the followings:

### Name

Medical diagnosis should be given priority for the name of adverse event. That is, if multiple symptoms, signs, and laboratory abnormal values can be called or attributed to the performance of a disease or damage, then this is regarded as one adverse event. If the diagnosis is not clear, symptoms/signs are used. When the later diagnosis is clear, the record is updated to replace the previous symptoms/signs.

### Starting Time

Take the “time of onset of symptoms” as the starting time of the adverse event. For those who progress from an adverse event to a serious adverse event, the occurrence time of the serious adverse event starts from the date when the adverse event escalates to a serious adverse event.

### Ending Time

The ending time of the adverse event is the time of recovery, stable condition, reasonable explanation and loss to follow-up. The time should be as accurate as possible to the year, month and day. If the information is incomplete, it should also be as accurate as possible to the year and month. If an adverse event that did not collect the ending time and was not the direct cause of death continued at the time of the subject's death, the ending time of the adverse event shall be left blank and the status shall be “persistence”. If an adverse event is judged to be the direct or primary cause of “death”, the ending time will be the time of the subject's death.

### Outcome

Restored/recovered; persisted; not restored/recovered; restored/recovered with sequelae; died; unknown.

### Concomitant Drugs

The concomitant drugs used for adverse events should be documented in the original medical record, and the name and use of the drugs should be clearly recorded (such as starting and ending time, dosage, route of administration, frequency of medication). It is recommended to indicate that the concomitant drugs are used to treat a specific adverse event/severe adverse event.

### Severity

### Relevance Evaluation

### 10.4 AE/SAE Follow-Up

All AE/SAE should be followed up until the end of the event, stable state, reasonable explanation, loss to follow-up, or death.

### 10.5 SAE Report

If an SAE occurs during the study, regardless of whether it is related to the study treatment, the investigator must immediately take appropriate treatment measures, and at the same time report all SAEs in writing to the sponsor within 24 hours of learning (email: clinicalsafety@leespharm.com; phone: 0551)-65310808-6508), and a detailed and written follow-up report should be provided promptly. The SAE first report and follow-up report should include the subject's identification code in the clinical trial, rather than the subject's real name, citizenship number, and address.

For reports involving deaths, the investigator should provide the sponsor and the ethics committee with any other information required, such as autopsy report and final medical report.

For all SAEs, the investigator is responsible for tracking and providing information to the sponsor in accordance with the reporting deadline specified above. In addition, the sponsor may request the investigator to quickly collect specific supplementary information. This information may be more detailed than that recorded in the SAE report form. Under normal circumstances, this information should include a detailed description of the SAE to allow for a complete medical evaluation of the event and an initial independent determination of possible causes. In addition, information about other possible causes, such as concomitant drugs and concomitant disease, should also be provided.

### 10.6 Pregnancy Events

Once a subject has a pregnancy event during the trial, the investigator should communicate with the subject scientifically and rigorously based on the medication information, inform her/him of the possible effects and risks of the study drug on the pregnant woman and fetus, and let the subject decide whether to terminate the pregnancy or continue the pregnancy.
For female subjects conceived during the trial, the investigator should first terminate the subjects' clinical trial after learning of the pregnancy event. If termination of pregnancy is stipulated in the protocol, the subject shall be notified and negotiated with regarding termination of pregnancy in the first time. If there is no scientific support that termination is necessary, the pregnancy needs to be closely followed up to its outcome (e.g., termination of pregnancy, delivery).
Male subjects whose sexual partners become pregnant during the trial will continue to participate in the trial provided that they report pregnancy events and follow their partner's termination of pregnancy or follow their partner's pregnancy to its outcome (e.g., termination of pregnancy, delivery).
Within 24 hours of confirming the subject's (or the subject's sexual partner's) pregnancy, the investigator should complete the initial pregnancy report form and report it according to SAE reporting procedures.
After receiving the pregnancy report form, the pharmacovigilance commissioner of the sponsor will keep the report for a long time if the pregnancy is successful; if there is fetal/neonatal congenital abnormality or malformation (SAE), spontaneous abortion (SAE), termination of pregnancy due to medical reasons (SAE), quick report should be adopted.

### 11 Data Management

### 11.1 Completion of Electronic Case Report Form

The electronic case report form is completed by the investigator, and the case report form must be completed by each enrolled patient. Before the start of the study, the data manager and relevant technical staff created an eCRF on the EDC platform based on the prepared paper case report form for the investigator to fill in. The data manager and the project leader carefully check the entry items of the eCRF and paper case report form and confirm the consistency. The entire EDC platform, including the database, eCRF and real-time logic verification procedures, should be tested several times and verified before being used.

At the same time, the data manager should work with the principal investigator to formulate the data range inspection and logical inspection content according to the range and interrelationship of each index value in the case report form. The corresponding computer program is written on the EDC platform to achieve the purpose of real-time logic check when filling in eCRF, and control the filling error of eCRF. At the same time, a back-end logic verification program is written to conduct back-end logic verification on the export line of the filled data.

### 11.2 Data Entry

Data entry and management are the responsibility of professional data management units. In this study, the EDC system is used, and the data entry process from paper CRF to the database is not required. After the start of the study, the investigator or assistant investigator will load the data into the electronic case report form on the EDC platform in a timely, complete, correct, and true manner based on the original data. After filling in the form, the investigator uses the password to sign the eCRF electronically.

### 11.3 Data Queries and Answers

The supervisor confirms on the EDC platform that all case report forms are correctly completed and consistent with the original data. If there are any errors or omissions, you can raise questions online at any time and ask the investigator to correct them in the eCRF in time. After the correction, the investigator will use the password to sign the corrected items electronically. The EDC platform keeps all traces of data changes, including the person who changed and the time of the change.

The data manager will conduct verification after exporting the data through the EDC platform, and inform the supervisor in time if any problem is found, and ask the investigator to answer it. The exchange of various questions and answers between them should use the query form function of the EDC platform, and all EDC query forms are stored in the system.

### 11.4 Data Monitoring

The supervisor needs to monitor whether the study is carried out in accordance with the study protocol. During the study process, the supervisor should check the informed consent and screening and inclusion of subjects in each study site regularly; confirm that all case report forms are filled in correctly and consistent with the original data, all errors or omissions have been corrected or noted, as well as signed and dated by the investigator; treatment, concomitant drugs, intermittent disease, loss to follow-up, inspection omissions, etc. of each subject should be confirmed and recorded; verify that the withdrawal and loss to follow-up of enrolled subjects have been indicated in the case report form; confirm that all adverse events are recorded and that serious adverse events are reported and recorded within the specified time; verify whether the study drugs are supplied, stored, distributed and recalled in accordance with relevant regulations and keep records accordingly.

### 11.5 Data Locking

At the end of data management, the principle investigator, sponsor, data manager and statistical analysts will confirm the statistical analysis population, and lock the database after solving all data queries. After all the data is locked, the data manager will import it into the designated database and submit it to the statistician for statistical analysis.

### 12 Statistic Analysis

### 12.1 Determination of Sample Size

The sample size estimation is based on the following assumptions:

Primary efficacy indicator: the proportion of patients whose corneal fluorescein staining score (ICSS) dropped by ≥1 point from baseline at Visit 5 (test eye). The test group is 30.7%; the control group is 18.5%. Use superiority test, and set alpha=0.025 (one-sided) and Beta=0.1. A total of 514 cases are required. Considering a 20% dropout, a total of 644 cases are required in the two groups.

### 12.2 Statistical Analysis Data Set

1) Full analysis set (FAS) includes all patients who are randomized to receive at least one dose of the drug and have at least one measurement of the Primary efficacy indicator after enrollment. In the absence of the primary efficacy indicator, last observation carried forward (LOCF) is used according to the intention to treat (ITT) principle.
2) Per protocol set (PPS) refers to the collection of cases that meet the inclusion criteria, fail to meet the exclusion criteria and complete the treatment plan, that is, all cases that are randomized, meet the requirements of the study protocol, have good compliance and complete the evaluation at Visit 5.
3) Safety set (SS): All subjects who have received at least one treatment and have at least one safety assessment after randomization. The incidence of adverse events is calculated with the number of cases in the safety data set as the denominator.

### 12.3 Statistical Analysis Plan

After the test protocol is determined, the statistical professionals are responsible for formulating the statistical analysis plan in consultation with the principal investigator. SAS 9.2 or above software is used for statistical analysis.

### 12.4 General Principle

A one-sided test is used for the efficacy analysis of the primary efficacy indicator, and a P value less than or equal to 0.025 is considered to be statistically significant. All other statistical tests are performed using a two-sided test (unless otherwise stated), and a P value less than or equal to 0.05 is considered to be statistically significant.

Quantitative indicators are described by the number of cases, mean, standard deviation, median, minimum and maximum. Classification indicators are described by the number of cases and percentages of each category.

### 12.5 Case Characteristics

### 12.5.1 Enrollment and Completion

Summarize the number of cases enrolled and completed in each site, and make a list of dropouts. χ² test or Fisher's exact probability method is used to compare the total dropout rate and the dropout rate due to adverse events between the two groups.

### 12.5.2 General Information and Baseline Characteristics

General information and baseline characteristics are analyzed using the FAS population. Baseline indicators including demographic characteristics of the two groups are compared by group t test or Wilcoxon test or χ² test to test the balance between the two groups. Describe the subject's concomitant drugs list.

### 12.6 Efficacy Analysis—Primary Efficacy Indicator

The proportion of patients whose corneal fluorescein staining score (ICSS) dropped by ≥1 point from baseline at Visit 5 (test eye), is analyzed by using the χ² test or Fisher's exact probability method to compare whether the proportion difference between the test group and the control group has statistical significance. Calculate the difference in the proportion of patients whose corneal fluorescein staining score (ICSS) dropped by ≥1 point from baseline at Visit 6 (test eye) between the test group and the control group, as well as the 97.5% confidence interval.

### Secondary Efficacy Indicators:

1) Mean changes in EDS scores from baseline at visits 3, 4 and 5 (both eyes); Use analysis of variance or Kruskal Wallis rank sum test for inter-group comparison.
2) Mean changes in VAS scores of the other 6 dry eye symptoms (burning sensation/stinging sensation, itching, foreign body sensation, discomfort, photophobia and pain) except for EDS from baseline at visits 3, 4 and 5 (both eyes); Use analysis of variance or Kruskal Wallis rank sum test for inter-group comparison.
3) Mean change of corneal fluorescein staining score (ICSS) from baseline at visits 3, 4 and 5 (test eye). Use analysis of variance or Kruskal Wallis rank sum test for inter-group comparison.
4) Mean change of corneal staining score (Oxford grading) from baseline at visits 3, 4 and 5 (test eye). Use analysis of variance or Kruskal Wallis rank sum test for inter-group comparison.
5) Mean change of break-up time (BUT) from baseline at visits 3, 4 and 5 (test eye). Use analysis of variance or Kruskal Wallis rank sum test for inter-group comparison.
6) Changes of Schirmer test value from baseline at visits 3, 4, and 5 (test eye). Use analysis of variance or Kruskal Wallis rank sum test for inter-group comparison.

### 12.7 Safety Analysis

The safety analysis is carried out with the safety data set (SS).

Make a list to describe the adverse events and adverse reactions of the two groups, including the number of adverse events/adverse reactions, severity, frequency and relationship with the study drug, the number of “normal to abnormal” or “anomalies worse” cases and the conversion rate by laboratory tests and vital signs before and after the trial. The chi-square test or Fisher's exact probability method is used for statistical analysis of the incidence of adverse events and adverse reactions. Cases where the study was discontinued due to adverse events and severe or serious adverse events occurred need to be explained.

### 13 Quality Control and Quality Assurance

During the course of this study, the clinical supervisor designated by the sponsor will make regular on-site inspection visits to the study site to ensure that all contents of the study protocol are strictly followed and the study data are filled in correctly.
1) Participating researchers must have the professional expertise, qualifications and abilities to undertake this clinical research, and have undergone unified training, unified recording methods and judgment standards.
2) The whole clinical study should be carried out in strict accordance with this study protocol.
3) The investigator should fill in the electronic case report form according to the requirements, and record the contents of the CRF truthfully, in detail, and carefully, so as to ensure the truthfulness and reliability of the content of the case report form.
4) All inspections adopt uniform inspection methods and judgment standards. The abnormal judgment standard of laboratory test shall be subject to the normal reference range of the inspection unit.
5) All observations and findings in clinical studies should be verified to ensure the reliability of data and ensure that conclusions in clinical studies are derived from original data. There are corresponding data management measures in the clinical study and data processing stages.
6) The sponsor assigns supervisors to regularly conduct on-site inspections of the clinical study unit to ensure that the contents of the study protocol are strictly adhered to and the study materials are filled in correctly and reliably.
7) The sponsor consigns inspectors not directly involved in the clinical trial to conduct a systematic review of the clinical trial related activities and documents to evaluate whether the trial is conducted in accordance with the trial plan, standard operating procedures and relevant regulations, and whether the trial data is recorded in a timely, true, accurate and complete manner.

### 14 Ethical Requirements and Informed Consent of Subjects

This clinical study has been approved by the National Medical Products Administration (formerly the State Food and Drug Administration)(Approval Document No. 2016L01275) before the start of the study. The clinical study protocol (including informed consent form, CRF, etc.) and other data provided to patients shall be reviewed and approved by the ethics committee of each participating unit before implementation.

The clinical study are carried out in strict accordance with the ethical guidelines for human medical research set out in the Declaration of Helsinki (2013 edition) and Good Clinical Practice (GCP), as well as relevant Chinese clinical study norms and regulations.

Before the start of the clinical study, the investigator introduction to the subjects enrolled in the study, including the purpose, process, method, performance of the drug, possible adverse reactions and risks, as well as the rights and obligations of the subjects. Each subject will be given a written informed consent form prior to enrollment to allow him/her sufficient time to consider whether or not to participate in the clinical trial. After obtaining informed consent from each subject and signing the informed consent form, the investigator begins clinical study.

### 15 Regulations on the Preservation and Ownership of Clinical Study Data

In order to ensure the evaluation and supervision of the National Medical Products Administration and the sponsor, the investigator should agree to maintain all research data, including the original data of all participating subjects, all signed informed consent forms, all CRFs, and and detailed records of drug distribution and recovery. The investigator should keep the clinical trial data for 5 years after the end of the clinical trial.

The ownership of the clinical study data belongs to Zhaoke (Guangzhou) Ophthalmology Pharmaceutical Limited, and the investigator shall not provide the data to any third party in any form without the written consent of the sponsor, except the National Medical Products Administration.

### 16 Results and Analysis

### 16.1 Patients and Treatments

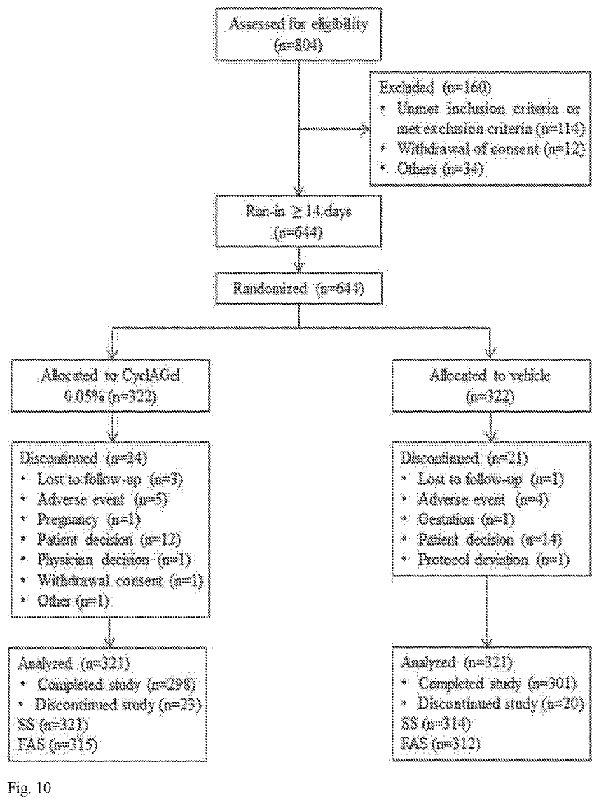

FIG. 10 presents the participant flowchart. During the study period, 804 participants were enrolled; 644 participants entered the 14-day run-in period. The participants were randomized 1:1 to the CyclAGel 0.05%/QD (n=322) or vehicle/QD (n=322) group. Finally, 298 and 301 participants in the CyclAGel and vehicle groups completed the trial. The dropout rate was 7%. The SS included 321 and 314 patients in the CyclAGel and vehicle groups, respectively. The FAS included 315 and 312 patients in the CyclAGel and vehicle groups, respectively. There were no statistically significant differences in the baseline characteristics between the two groups in terms of age (45.0±13.7 vs 44.5±14.2 years). sex (male: 18.1% vs 16.3%), body mass index (22.9±3.4 vs 22.5±2.9 kg/m2), DED duration (median: 4.3 vs 5.9 months, Mann-Whitney U-test, P=0.3586), DED affected side, and study eye (all P>0.05). (Table 15-21)

TABLE 15
Distribution of Subjects (All subjects)

| | CsA | | | |
|---|---|---|---|---|
| | Ophthalmic | | | |
| | Gel | vehicle | Total | |
| | N = 322 | N = 322 | N = 644 | |
| Characteristic | n (%) | n (%) | n (%) | P value |
| Patients | 322 (100%) | 322 (100%) | 644 (100%) | |
| randomized | | | | |
| to enrollment | | | | |
| Patients | 321 (99.7%) | 314 (97.5%) | 635 (98.6%) | |
| administering | | | | |
| at least one dose | | | | |
| of test drug | | | | |
| Patients | 298 (92.5%) | 301 (93.5%) | 599 (93.0%) | |
| completing | | | | |
| the study | | | | |
| Patients | 24 (7.5%) | 21 (6.5%) | 45 (7.0%) | 0.6429 [1] |
| discontinuing | | | | |
| the study | | | | |

Reason for discontinuation

| | | | | |
|---|---|---|---|---|
| Loss to follow-up | 3 (0.9%) | 1 (0.3%) | 4 (0.6%) | |
| Adverse events | 5 (1.6%) | 4 (1.2%) | 9 (1.4%) | >0.9999 [2] |
| Pregnancy | 1 (0.3%) | 1 (0.3%) | 2 (0.3%) | |
| Subject choice | 12 (3.7%) | 14 (4.3%) | 26 (4.0%) | |
| Protocol | 0 | 1 (0.3%) | 1 (0.2%) | |
| deviation | | | | |
| Investigator | 1 (0.3%) | 0 | 1 (0.2%) | |
| discretion | | | | |
| ICS withdrawn | 1 (0.3%) | 0 | 1 (0.2%) | |
| Others | 1 (0.3%) | 0 | 1 (0.2%) | |

Percentage is calculated based on the patients randomly enrolled in each group.
[1] P value is based on χ2 test.
[2] P value is based on Fisher's Exact test.

The main baseline characteristics of the patients are shown in Tables 14-21. The demographic data and baseline ocular characteristics of the vehicle group were generally consistent with those of the cyclosporin A ophthalmic gel group.

TABLE 16
Demographic information (randomized population)

| | | | |
|---|---|---|---|
| Cyclosporin A | | | |
| Ophthalmic Gel | Vehicle | Total | |
| (N = 322) | (N = 322) | (N = 644) | P-value |

Age (years)

| | | | | | |
|---|---|---|---|---|---|
| Number of cases | 322 | 322 | 644 | 0.5742 | [1] |

| | | | |
|---|---|---|---|
| Mean value | 45.0 (13.58) | 44.3 (14.11) | 44.6 (13.84) |
| (standard | | | |
| deviation) | | | |
| Median | 45.0 | 45.0 | 45.0 |
| Min-Max | 18-78 | 18-77 | 18-78 |

Gender, n (%)

| | | | | | |
|---|---|---|---|---|---|
| Male | 57 (17.7%) | 52 (16.1%) | 109 (16.9%) | 0.5993 | [2] |

| | | | |
|---|---|---|---|
| Female | 265 (82.3%) | 270 (83.9%) | 535 (83.1%) |

Height (cm)

| | | | | | |
|---|---|---|---|---|---|
| Number of cases | 322 | 322 | 644 | 0.5593 | [1] |

| | | | |
|---|---|---|---|
| Mean value | 162.09 (7.372) | 162.33 (7.673) | 162.21 (7.519) |
| (standard | | | |
| deviation) | | | |
| Median | 160.80 | 161.00 | 161.00 |
| Min-Max | 142.6-186.5 | 138.0-189.2 | 138.0-189.2 |

Weight (kg)

| | | | | | |
|---|---|---|---|---|---|
| Number of cases | 322 | 322 | 644 | 0.2298 | [1] |

| | | | |
|---|---|---|---|
| Mean value | 60.45 (11.152) | 59.16 (9.618) | 59.80 (10.425) |
| (standard | | | |
| deviation) | | | |
| Median | 58.65 | 57.70 | 58.00 |
| Min-Max | 39.1-119.3 | 37.1-104.0 | 37.1-119.3 |

Body mass index (kg/m²)

| | | | | | |
|---|---|---|---|---|---|
| Number of cases | 322 | 322 | 644 | 0.0937 | [1] |
| Mean value | 22.93 (3.341) | 22.40 (2.883) | 22.66 (3.129) | 22.93 | (3.341) |

| | | | | |
|---|---|---|---|---|
| (standard | | | | |
| deviation) | | | | |
| Median | 22.58 | 22.31 | 22.38 | 22.58 |
| Min-Max | 16.4-37.1 | 15.8-31.4 | 15.8-37.1 | 16.4-37.1 |

Body mass index = body weight (kg)/[height (cm)/100]².
Percentages were calculated based on the number of subjects in each treatment group among all randomly enrolled subjects.
[1] P values are based on rank sum test.
[2] P values are based on chi-square tests.

TABLE 17
Demographic information (full analysis set)

| | | | |
|---|---|---|---|
| Cyclosporin A | | | |
| Ophthalmic Gel | Vehicle | Total | |
| (N = 322) | (N = 322) | (N = 644) | P-value |

Age (years)

| Number of cases | 315 | | 312 | | 627 | | 0.7023 [1] |
|---|---|---|---|---|---|---|---|
| Mean value | 45.0 | (13.65) | 44.5 | (14.22) | 44.7 | (13.93) | |

| | | | |
|---|---|---|---|
| (standard | | | |
| deviation) | | | |
| Median | 45.0 | 45.0 | 45.0 |
| Min-Max | 18-78 | 18-77 | 18-78 |

Gender, n (%)

| | | | | | | | |
|---|---|---|---|---|---|---|---|
| Male | 57 | (18.1%) | 51 | (16.3%) | 108 | (17.2%) | 0.5620 [2] |
| Female | 258 | (81.9%) | 261 | (83.7%) | 519 | (82.8%) | |

Height (cm)

| Number of cases | 315 | | 312 | | 627 | | 0.5787 [1] |
|---|---|---|---|---|---|---|---|
| Mean value | 162.13 | (7.439) | 162.35 | (7.714) | 162.24 | (7.572) | |

| | | | |
|---|---|---|---|
| (standard | | | |
| deviation) | | | |
| Median | 161.00 | 161.15 | 161.00 |
| Min-Max | 142.6-186.5 | 138.0-189.2 | 138.0-189.2 |

Weight (kg)

| Number of cases | 315 | | 312 | | 627 | | 0.3851 [1] |
|---|---|---|---|---|---|---|---|
| Mean value | 60.44 | (11.242) | 59.32 | (9.625) | 59.88 | (10.475) | |

| | | | |
|---|---|---|---|
| (standard | | | |
| deviation) | | | |
| Median | 58.60 | 58.00 | 58.00 |
| Min-Max | 39.1-119.3 | 37.1-104.0 | 37.1-119.3 |

Body mass index (kg/m²)

| Number of cases | 315 | | 312 | | 627 | | 0.1954 [1] |
|---|---|---|---|---|---|---|---|
| Mean value | 22.91 | (3.358) | 22.46 | (2.893) | 22.69 | (3.141) | |

| | | | |
|---|---|---|---|
| (standard | | | |
| deviation) | | | |
| Median | 22.58 | 22.38 | 22.46 |
| Min-Max | 16.4-37.1 | 15.8-31.4 | 15.8-37.1 |

Body mass index = body weight (kg)/[height (cm)/100]².
Percentages were calculated based on the number of subjects in each treatment group among all randomly enrolled subjects.
[1] P values are based on rank sum test.
[2] P values are based on chi-square tests.

TABLE 18
Baseline disease characteristics (full analysis set)

| | | | |
|---|---|---|---|
| Cyclosporin A | | | |
| Ophthalmic Gel | Vehicle | Total | |
| (N = 315) | (N = 312) | (N = 627) | P-value |

Time from first diagnosis of dry eye disease to randomization grouping (months)

| Number of cases | 312 | | 309 | | 621 | | 0.3586 [2] |
|---|---|---|---|---|---|---|---|
| Mean value | 21.00 | (40.648) | 18.99 | (31.307) | 20.00 | (36.286) | |

| | | | |
|---|---|---|---|
| (standard deviation) | | | |
| Median | 4.25 | 5.90 | 4.70 |
| Min-Max | 0.5-476.3 | 0.5-247.7 | 0.5-476.3 |

Location of dry eye disease, n (%)

| | | | | | | | |
|---|---|---|---|---|---|---|---|
| Right eye | 2 | (0.6%) | 4 | (1.3%) | 6 | (1.0%) | 0.6972 [4] |
| Left eye | 4 | (1.3%) | 3 | (1.0%) | 7 | (1.1%) | |
| Both eye | 309 | (98.1%) | 305 | (97.8%) | 614 | (97.9%) | |

Baseline EDS score

| Number of cases | 315 | | 312 | | 627 | | 0.4086 [2] |
|---|---|---|---|---|---|---|---|
| Mean value | 65.8 | (13.67) | 65.0 | (13.96) | 65.4 | (13.81) | |

| | | | |
|---|---|---|---|
| (standard deviation) | | | |
| Median | 65.0 | 63.0 | 64.0 |
| Min-Max | 41-99 | 40-100 | 40-100 |

Baseline burning/pinching sensation score

| Number of cases | 315 | | 312 | | 627 | | 0.7163 [2] |
|---|---|---|---|---|---|---|---|
| Mean value | 36.2 | (27.03) | 35.6 | (27.31) | 35.9 | (27.15) | |

| | | | |
|---|---|---|---|
| (standard deviation) | | | |
| Median | 34.0 | 34.5 | 34.0 |
| Min-Max | 0-96 | 0-97 | 0-97 |

Baseline pruritus score

| Number of cases | 315 | | 312 | | 627 | | 0.2205 [2] |
|---|---|---|---|---|---|---|---|
| Mean value | 35.9 | (27.49) | 33.4 | (27.26) | 34.7 | (27.39) | |

| | | | |
|---|---|---|---|
| (standard | | | |
| deviation) | | | |
| Median | 31.0 | 28.0 | 30.0 |
| Min-Max | 0-98 | 0-95 | 0-98 |

Baseline foreign body sensation score

| Number of cases | 315 | | 312 | | 627 | | 0.2070 [2] |
|---|---|---|---|---|---|---|---|
| Mean value | 43.8 | (27.91) | 41.1 | (28.55) | 42.5 | (28.24) | |

| | | | |
|---|---|---|---|
| (standard deviation) | | | |
| Median | 44.0 | 41.0 | 43.0 |
| Min-Max | 0-99 | 0-98 | 0-99 |

Baseline discomfort score

| Number of cases | 315 | | 312 | | 627 | | 0.6946 [2] |
|---|---|---|---|---|---|---|---|
| Mean value | 49.7 | (26.61) | 50.5 | (27.16) | 50.1 | (26.87) | |

| | | | |
|---|---|---|---|
| (standard deviation) | | | |
| Median | 52.0 | 54.0 | 53.0 |
| Min-Max | 0-98 | 0-96 | 0-98 |

Baseline photophobia score

| Number of cases | 315 | | 312 | | 627 | | 0.1575 [2] |
|---|---|---|---|---|---|---|---|
| Mean value | 43.8 | (28.07) | 40.7 | (29.84) | 42.3 | (28.98) | |

| | | | |
|---|---|---|---|
| (standard deviation) | | | |
| Median | 44.0 | 41.5 | 43.0 |
| Min-Max | 0-99 | 0-100 | 0-100 |

Baseline pain score

| Number of cases | 315 | | 312 | | 627 | | 0.1471 [2] |
|---|---|---|---|---|---|---|---|
| Mean value | 27.9 | (25.65) | 26.0 | (25.90) | 26.9 | (25.77) | |

| | | | |
|---|---|---|---|
| (standard deviation) | | | |
| Median | 19.0 | 16.5 | 18.0 |
| Min-Max | 0-98 | 0-98 | 0-98 |

Time from first dry eye diagnosis to randomization grouping (months) = (date of randomization − date of first diagnosis + 1)/30.4375, with 1 decimal place retained.
Baseline was defined as the last non-missing assessment/examination prior to the first trial drug administration. Percentages were calculated based on the number of subjects in each treatment group in the full analysis set.
[1] P values are based on t-tests.
[2] P values are based on rank sum test.
[3] P values are based on chi-square tests.
[4] P-values are based on Fisher's Exact test.

TABLE 19
Baseline ocular characteristics of study eyes (full analysis set)

| | | | |
|---|---|---|---|
| Cyclosporin A | | | |
| Ophthalmic Gel | Vehicle | Total | |
| (N = 315) | (N = 312) | (N = 627) | P-value |

Study eyes, n (%)

| | | | | | | | |
|---|---|---|---|---|---|---|---|
| Right eye | 110 | (34.9%) | 117 | (37.5%) | 227 | (36.2%) | 0.5016 [3] |
| Left eye | 205 | (65.1%) | 195 | (62.5%) | 400 | (63.8%) | |

Baseline ICSS score

| Number of cases | 315 | | 312 | | 627 | | 0.7227 [2] |
|---|---|---|---|---|---|---|---|
| Mean value | 3.0 | (0.79) | 3.0 | (0.76) | 3.0 | (0.77) | |

| | | | |
|---|---|---|---|
| (standard deviation) | | | |
| Median | 3.0 | 3.0 | 3.0 |
| Min-Max | 2-4 | 2-4 | 2-4 |

Baseline keratoconjunctival staining score (Oxford grading)

| Number of cases | 314 | | 311 | | 625 | | 0.4595 [2] |
|---|---|---|---|---|---|---|---|
| Mean value | 6.0 | (3.68) | 6.3 | (3.85) | 6.1 | (3.76) | |

| | | | |
|---|---|---|---|
| (standard deviation) | | | |
| Median | 5.0 | 5.0 | 5.0 |
| Min-Max | 0-15 | 0-15 | 0-15 |

Baseline BUT measurement value

| Number of cases | 315 | | 312 | | 627 | | 0.4087 [2] |
|---|---|---|---|---|---|---|---|
| Mean value | 3.319 | (1.5730) | 3.402 | (1.5412) | 3.360 | (1.5566) | |

| | | | |
|---|---|---|---|
| (standard deviation) | | | |
| Median | 2.960 | 3.115 | 3.030 |
| Min-Max | 0.00-9.23 | 0.00-9.12 | 0.00-9.23 |

Baseline Schirmer test values

| Number of cases | 315 | | 312 | | 627 | | 0.8044 [2] |
|---|---|---|---|---|---|---|---|
| Mean value | 3.7 | (2.44) | 3.8 | (2.53) | 3.7 | (2.49) | |

| | | | |
|---|---|---|---|
| (standard deviation) | | | |
| Median | 3.0 | 3.0 | 3.0 |
| Min-Max | 0-9 | 0-9 | 0-9 |

Baseline was defined as the last non-missing assessment/check before the first trial drug was administered.
Percentages were calculated based on the number of subjects in each treatment group in the full analysis set.
[1] P values are based on t-tests,
[2] P values are based on rank sum test.
[3] P values are based on chi-square tests.
[4] P values are based on Fisher's Exact test.

TABLE 20
Baseline ocular characteristics of the contralateral eye (full analysis set)

| | | | |
|---|---|---|---|
| Cyclosporin A | | | |
| Ophthalmic Gel | Vehicle | Total | |
| (N = 315) | (N = 312) | (N = 627) | P-value |

Contralateral eye, n (%)

| | | | | | | | |
|---|---|---|---|---|---|---|---|
| Right eye | 110 | (34.9%) | 117 | (37.5%) | 227 | (36.2%) | 0.5016 [3] |
| Left eye | 205 | (65.1%) | 195 | (62.5%) | 400 | (63.8%) | |

Baseline ICSS score

| Number of cases | 313 | | 309 | | 622 | | 0.7864 [2] |
|---|---|---|---|---|---|---|---|
| Mean value | 2.3 | (1.05) | 2.2 | (1.08) | 2.2 | (1.06) | |

| | | | |
|---|---|---|---|
| (standard deviation) | | | |
| Median | 2.0 | 2.0 | 2.0 |
| Min-Max | 0-4 | 0-4 | 0-4 |

Baseline BUT measurement value

| Number of cases | 315 | | 312 | | 627 | | 0.9086 [2] |
|---|---|---|---|---|---|---|---|
| Mean value | 3.452 | (1.6455) | 3.440 | (1.6493) | 3.446 | (1.6461) | |

| | | | |
|---|---|---|---|
| (standard deviation) | | | |
| Median | 3.170 | 3.170 | 3.170 |
| Min-Max | 0.00-10.01 | 0.00-10.12 | 0.00-10.12 |

Baseline Schirmer test values

| Number of cases | 313 | | 310 | | 623 | | 0.8054 [2] |
|---|---|---|---|---|---|---|---|
| Mean value | 4.0 | (3.18) | 4.1 | (3.42) | 4.0 | (3.30) | |

| | | | |
|---|---|---|---|
| (standard deviation) | | | |
| Median | 3.0 | 3.0 | 3.0 |
| Min-Max | 0-19 | 0-18 | 0-19 |

Baseline was defined as the last non-missing assessment/check before the first trial drug was administered.
Percentages were calculated based on the number of subjects in each treatment group in the full analysis set.
[1] P values are based on t-tests.
[2] P values are based on rank sum test.
[3] P values are based on chi-square tests.
[4] P values are based on Fisher's Exact test.

TABLE 21
Baseline Characteristics of the Participants

| | CyclAGel | | | | |
|---|---|---|---|---|---|
| | 0.05% | | Vehicle | | |
| Characteristics | (n = 315) | | (n = 312) | | P |
| Age (years) | 45.0 ± 13.7 | | 44.5 ± 14.2 | | 0.7023 |
| Sex (male) | 57 | (18.1%) | 51 | (16.3%) | 0.5620 |
| BMI (kg/m²) | 22.9 ± 3.4 | | 22.5 ± 2.9 | | 0.1954 |
| DED duration (months)^{#} | 4.3 | (0.5-476.3) | 5.9 | (0.5-247.7) | 0.3586 |
| DED affected side | | | | | 0.6972 |
| Right eye | 2 | (0.6%) | 4 | (1.3%) | |
| Left eye | 4 | (1.3%) | 3 | (1.0%) | |
| Both eyes | 309 | (98.1%) | 305 | (98.0%) | |
| Studied eye | | | | | 0.5016 |
| Left eye | 110 | (34.9%) | 117 | (37.5%) | |
| Right eye | 205 | (65.1%) | 195 | (62.5%) | |
| Ongoing ocular diseases | 119 | (37.8%) | 128 | (41.0%) | — |
| Ongoing non-ocular | 156 | (49.5%) | 140 | (44.9%) | — |
| diseases | | | | | |
| Ocular concomitant | 7 | (2.2%) | 5 | (1.6%) | — |
| medications | | | | | |
| Non-ocular concomitant | 120 | (38.1%) | 109 | (34.9%) | — |
| medications | | | | | |

Notes:
^{#}Duration of disease was defined as the time from first dry eye diagnosis to randomizedgrouping.
Abbreviations: BMI, body mass index; DED, dry eye disease.

### 16.2 Efficacy Evaluation

Drug group was an independent variable, EDS at baseline was a covariate, and EDS at visit 5 was a dependent variable for ANOVA. This analysis was performed to identify least squares mean differences of the treatment and control groups from the model, and were presented with P values and two-sided 95% CIs. Moreover, it was evaluated whether a dose response relationship could be observed. The primary efficacy end point was baseline changes in EDS (VAS) at week 12 with CysA Gel. The changes from baseline to weeks 4 and 8 were reported as secondary efficacy end points. Other key end points included changes from baseline in 6 other dry eye symptoms, BUT, cornea staining scores, and a nonanesthetic Schirmer test score at weeks 4, 8, and 12. The differences between the 2 groups were tested by using ANOVA or Kruskale Wallis rank sum tests.

The primary safety end point was the incidence of AEs in patients who had received at least 1 dose of trial drug. Clinical laboratory tests, check of vital signs, and a physical examination were also repeated at the end of the trial, or when patients dropped out, to evaluate the changes in general health status compared with those at screening. Drug tolerance was also included in the safety analysis.

### Primary Efficacy Endpoint

Among the FAS subjects, a total of 232 subjects (73.7%) in the cyclosporine A ophthalmic gel group visited the 5 study eyes (below) with a decrease of ≥1 point from baseline in corneal fluorescein staining score (ICSS) compared to the vehicle group and 166 subjects (53.2%) in the vehicle group. The inter-rater difference and corresponding 97.5% one-sided confidence interval for comparison with the vehicle group was 20.4% (13.1%, +∞), with a one-sided P value <0.0001. P value <0.025, lower limit of 97.5% CI >0%, showing that at visit 5, the proportion of subjects in the cyclosporine A ophthalmic gel group with a decrease of ≥1 point in corneal fluorescein staining score (ICSS) from baseline was significantly greater than that in the vehicle group. The proportion was significantly greater than in the vehicle group. Treatment with cyclosporine A ophthalmic gel for 84 days significantly reduced (lower) corneal fluorescein staining scores in patients with moderate to severe dry eyes.

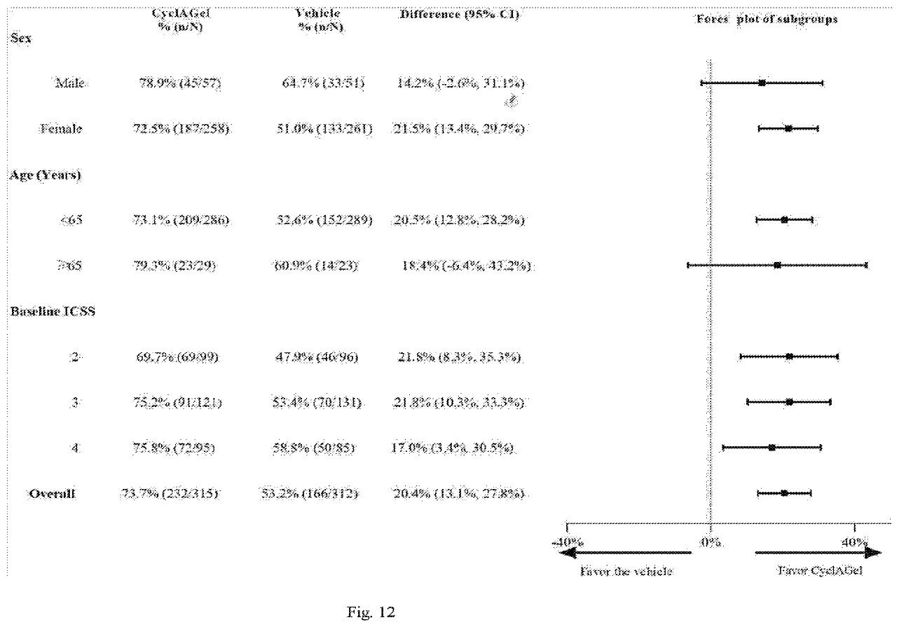

The proportion of subjects with at least a 1-point improvement in ICSS from baseline to day 84 was 73.7% (232/315) in the CyclAGel group vs 53.2% (166/312) in the vehicle group (P<0.0001), achieving the study primary endpoint. On days 14 and 42, the proportions of subjects with at least a 1-point improvement in ICSS from baseline were also significantly higher in the CyclAGel group compared with the vehicle group (54.5% vs 44.2%, P=0.0052; 69.2% vs 52.8%, P<0.0001; FIG. 11, Table 24). According to the subgroup analyses, females <65 years were more likely to benefit from CyclAGel (FIG. 12).

At days 14, 42, and 84, the percentage of subjects with iCSS decreased by more than 1 points and more than 2 points in the CyclAGel group were significantly lower than the control group. The onset time of CsA gel is within 14 days. (FIGS. 11, 15, 16). The keratocconjunctival staining score decreased significantly from baseline, at days 14, 42, and 84, which was statistically significant. (FIG. 16).

### Secondary Efficacy Endpoint

In the cyclosporine A ophthalmic gel group, all VAS scores for dry eye symptoms were lower at visit 4 and visit 5; ICSS and keratoconjunctival staining scores (Oxford grading) were lower at all visits (3, 4, 5); BUT measurements and Schirmer's test scores (Oxford grading) were lower at all visits and keratoconjunctival staining scores (Oxford grading); and BUT measurements and Schirmer's test scores (Oxford grading) were higher at all visits (3, 4, 5). The values of BUT and Schirmer's test were all increased. All secondary efficacy indicator test values were increased in the vehicle group. All secondary efficacy indicators improved from baseline in the vehicle group. Inter-group comparisons between the cyclosporine A ophthalmic gel group and the vehicle group showed that the change from baseline in the ICSS, the Oxford Conjunctival Staining Score (OCS), the Oxford Conjunctival Staining Score (OCS), and the Oxford Conjunctival Staining Score (OCS) Schirmer test values were numerically higher in the cyclosporine A ophthalmic gel group than in the vehicle group.

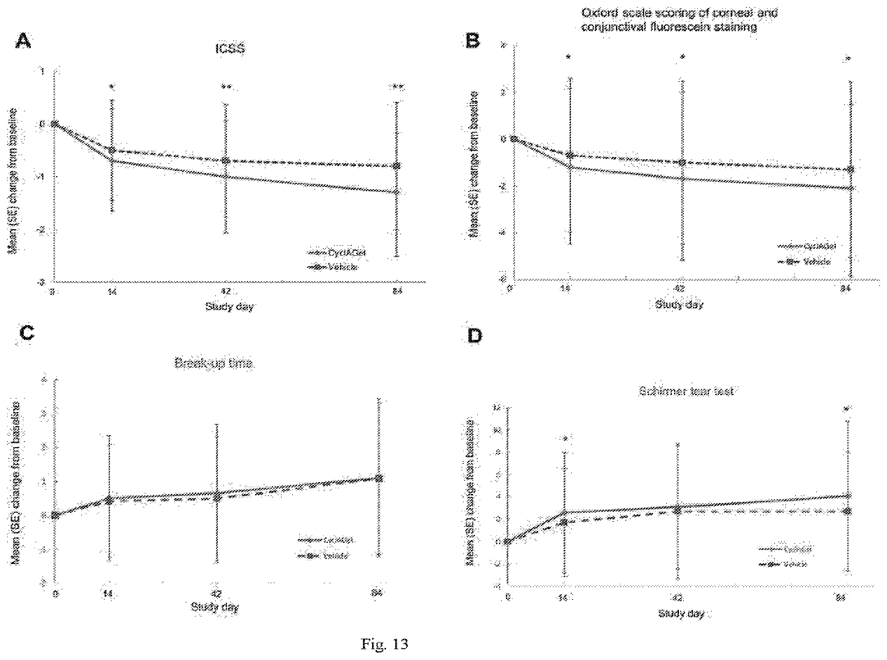

The secondary endpoints are presented in FIG. 13, Tables 22 and 23. The mean changes from baseline in ICSS and Oxford scale scoring of corneal and conjunctival fluorescein staining on days 14, 42, and 84 were significantly higher in the CycAGel group than in the vehicle group (all P<0.05). The mean changes from baseline in the Schirmer tear test on days 14 and 84 were higher in the CyclAGel group than in the vehicle group (both P<0.05), but the difference was not significant on day 42. No statistically significant differences between groups regarding the mean changes of BUT from baseline to days 14, 42, and 84 were observed (all P>0.05). Concerning symptoms, the EDS on day 84 was significantly decreased from baseline in both groups (mean value. −29 in the CyclAGel group vs −31 in the vehicle group, P=0.346). Other symptoms, including itching, foreign body sensation, photophobia, burning/stinging sensation, discomfort, and pain, were significantly decreased at day 84 compared to baseline in both groups. No superiority of CyclAGel over the vehicle regarding binocular symptom scores was observed at any visit.

TABLE 22
Changes from Baseline in DED Signs of
the Study Eye at Days 14, 42, and 84

| | CyclAGel | | | Vehicle | | |
|---|---|---|---|---|---|---|
| DED | (n = 315) | | | (n = 312) | | |
| Signs | Day 14 | Day 42 | Day 84 | Day 14 | Day 42 | Day 84 |
| ICSS | −0.7 ± | −1.0 ± | −1.3 ± | −0.5 ± | −0.7 ± | −0.8 ± |
| | 0.99 | 1.06 | 1.13 | 0.95* | 1.06* | 1.21* |
| Oxford | −1.2 ± | −1.7 ± | −2.1 ± | −0.7 ± | −1.0 ± | −1.3 ± |
| scale | 3.35 | 3.72 | 3.57 | 3.29* | 3.47* | 3.75* |
| scoring of | | | | | | |
| corneal | | | | | | |
| and | | | | | | |
| conjunctival | | | | | | |
| fluorescein | | | | | | |
| staining | | | | | | |
| BUT (s) | 0.501 ± | 0.658 ± | 1.105 ± | 0.421 ± | 0.498 ± | 1.093 ± |
| | 1.8389 | 2.0332 | 2.3108 | 1.6580 | 1.8054 | 2.2247 |
| Schirmer | 2.6 ± | 3.1 ± | 4.1 ± | 1.7 ± | 2.7 ± | 2.7 ± |
| tear test | 5.39 | 5.57 | 6.71 | 4.79* | 6.05 | 5.34* |
| (mm/5 min) | | | | | | |

Notes:
*P < 0.05 vs CyclAGel group.
Abbreviations: DED, dry eye disease; ICSS, inferior corneal staining score; BUT, break-up time.

TABLE 23
Changes in Binocular Symptom Scores from Baseline at Days 14, 42, and 84

| Binocular | CyclAGel | | | Vehicle | | |
|---|---|---|---|---|---|---|
| Symptom | (n = 315) | | | (n = 312) | | |
| Scores | Day 14 | Day 42 | Day 84 | Day 14 | Day 42 | Day 84 |
| EDS | −9 | −20 | −29 | −9 | −23 | −31 |
| | (−92 to 39) | (−93 to 32) | (−95 to 39) | (−92 to 38) | (−98 to 39) | (−94 to 23) |
| Burning/tingling | 0 | −2 | −5 | −2 | −6 | −12 |
| sensation | (−94 to 79) | (−95 to 93) | (−95 to 88) | (−95 to 81)* | (−97 to 73)* | (−86 to 72)* |
| Itching | −1.5 | −5 | −7.5 | −1 | −4 | −9 |
| | (−93 to 88) | (−96 to 82) | (−96 to 77) | (−83 to 83) | (−89 to 70) | (−94 to 72) |
| Foreign body | −2 | −7 | −14 | −3 | −6 | −15 |
| sensation | (−90 to 63) | (−91 to 60) | (−89 to 65) | (−80 to 83) | (−86 to 85) | (−91 to 71) |
| Discomfort | −2 | −11 | −16.5 | −5 | −12 | −23 |
| | (−86 to 93) | (−92 to 81) | (−92 to 81) | (−95 to 76)* | (−96 to 71) | (−92 to 85)* |
| Photophobia | 0 | −7 | −10 | −3 | −6 | −13 |
| | (−97 to 58) | (−98 to 84) | (−98 to 63) | (−81 to 79)* | (−85 to 85) | (−77 to 74) |
| Pain | 0 | −2 | −3 | 0 | −2 | −5 |
| | (−96 to 84) | (−97 to 90) | (−98 to 89) | (−82 to 80)* | (−76 to 61) | (−88 to 53)* |

Notes:
*P < 0.05 vs CyclAGel group.
Abbreviation: EDS, eye dryness score.

TABLE 24
Summary of the proportion of subjects with a ≥1-point decrease from baseline in
the study eye's corneal fluorescein staining score (ICSS) (full analysis set)

| | |
|---|---|
| Cyclosporine A | |
| Ophthalmic Gel | Vehicle |
| (N = 315) | (N = 312) |

Visit 3 (OC)

| Number of evaluable cases | 314 | | 310 | |
|---|---|---|---|---|
| ICSS decreased ≥1 point from baseline, n (%) | 171 | (54.5%) | 137 | (44.2%) |

| | | |
|---|---|---|
| 95% two-sided confidence interval [1] | (48.8%, 60.1%) | (38.6%, 49.9%) |

Comparison with vehicle

| | | |
|---|---|---|
| Ratio difference between groups | 10.3% | (2.5%, +∞) |
| (97.5% one-sided confidence interval) [2] | | |
| Ratio difference between groups | 10.3% | (2.5%, 18.1%) |

| | |
|---|---|
| (95% two-sided confidence interval) [2] | |
| Unilateral P-value [3] | 0.0052 |

Visit 4 (OC)

| Number of evaluable cases | 305 | | 303 | |
|---|---|---|---|---|
| ICSS decreased ≥1 point from baseline, n (%) | 211 | (69.2%) | 160 | (52.8%) |

| | | |
|---|---|---|
| 95% two-sided confidence interval [1] | (63.7%, 74.3%) | (47.0%, 58.5%) |

Comparison with vehicle

| | | |
|---|---|---|
| Ratio difference between groups | 16.4% | (8.7%, +∞) |
| (97.5% one-sided confidence interval) [2] | | |
| Ratio difference between groups | 16.4% | (8.7%, 24.0%) |

| | |
|---|---|
| (95% two-sided confidence interval) [2] | |
| Unilateral P-value [3] | <0.0001 |

Visit 5 (OC)

| Number of evaluable cases | 298 | | 301 | |
|---|---|---|---|---|
| ICSS decreased ≥1 point from baseline, n (%) | 223 | (74.8%) | 164 | (54.5%) |

| | | |
|---|---|---|
| 95% two-sided confidence interval [1] | (69.5%, 79.7%) | (48.7%, 60.2%) |

Comparison with vehicle

| | | |
|---|---|---|
| Ratio difference between groups | 20.3% | (12.9%, +∞) |
| (97.5% one-sided confidence interval) [2] | | |
| Ratio difference between groups | 20.3% | (12.9%, 27.8%) |

| | |
|---|---|
| (95% two-sided confidence interval) [2] | |
| Unilateral P-value [3] | <0.0001 |

Visit 5 (NR/LOCF) [4]

| Number of evaluable cases | 315 | | 312 | |
|---|---|---|---|---|
| ICSS decreased ≥1 point from baseline, n (%) | 232 | (73.7%) | 166 | (53.2%) |

| | | |
|---|---|---|
| 95% two-sided confidence interval [1] | (68.4%, 78.4%) | (47.5%, 58.8%) |

Comparison with vehicle

| Ratio difference between groups | 20.4% | (13.1%, +∞) |
|---|---|---|
| (97.5% one-sided confidence interval) [2] | | |
| Ratio difference between groups | 20.4% | (13.1%, 27.8%) |

| | |
|---|---|
| (95% two-sided confidence interval) [2] | |
| Unilateral P-value [3] | <0.0001 |

LOCF = last result carryover, NR = no answer, OC = actual observation.
Percentages were calculated based on the number of evaluable subjects in each visit for each treatment group in the full analysis set. [symbol]
1] Binomial distribution based two-sided exact confidence intervals (Clopper-Pearson confidence intervals). [symbol]
2] Asymptotic confidence intervals for the differences in rates between groups obtained based on the normal distribution approximation method. [symbol]
3] Based on the chi-square test or Fisher's Exact test. [symbol]
4] Subjects were considered ″non-responders″ if they withdrew early from the study due to poor treatment efficacy or ocular-related adverse events. If a subject withdrew early from the study for other reasons, the subject's last valid post-baseline ICSS score (i.e., LOCF) prior to early withdrawal was used in the calculation of the primary efficacy endpoint. [symbol]
indicates data missing or illegible when filed

TABLE 25
Summary of the proportion of subjects with a ≥1-point decrease from baseline in the
study eye's corneal fluorescein staining score (ICSS) (consistent with the protocol set)
Ophthalmic Gel

| | |
|---|---|
| Cyclosporine A | |
| Ophthalmic Gel | Vehicle |
| (N = 287) | (N = 295) |

Visit 3 (OC)

| Number of evaluable cases | 287 | | 295 | |
|---|---|---|---|---|
| ICSS decreased ≥1 point from baseline, n (%) | 155 | (54.0%) | 132 | (44.7%) |

| | | |
|---|---|---|
| 95% two-sided confidence interval [1] | (48.1%, 59.9%) | (39.0%, 50.6%) |

Comparison with vehicle

| Ratio difference between groups | 9.3% | (1.2%, +∞) |
|---|---|---|
| (97.5% one-sided confidence interval) [2] | | |
| Ratio difference between groups | 9.3% | (1.2%, 17.4%) |

| | |
|---|---|
| (95% two-sided confidence interval) [2] | |
| Unilateral P-value [3] | 0.0127 |

Visit 4 (OC)

| Number of evaluable cases | 287 | | 294 | |
|---|---|---|---|---|
| ICSS decreased ≥1 point from baseline, n (%) | 198 | (69.0%) | 157 | (53.4%) |

| | | |
|---|---|---|
| 95% two-sided confidence interval [1] | (63.3%, 74.3%) | (47.5%, 59.2%) |

Comparison with vehicle

| Ratio difference between groups | 15.6% | (7.8%, +∞) |
|---|---|---|
| (97.5% one-sided confidence interval) [2] | | |
| Ratio difference between groups | 15.6% | (7.8%, 23.4%) |

| | |
|---|---|
| (95% two-sided confidence interval) [2] | |
| Unilateral P-value [3] | <0.0001 |

Visit 5 (OC)

| Number of evaluable cases | 287 | | 295 | |
|---|---|---|---|---|
| ICSS decreased ≥1 point from baseline, n (%) | 217 | (75.6%) | 161 | (54.6%) |

| | | |
|---|---|---|
| 95% two-sided confidence interval [1] | (70.2%, 80.5%) | (48.7%, 60.4%) |

Comparison with vehicle

| Ratio difference between groups | 21.0% (13.5%, +∞) | |
|---|---|---|
| (97.5% one-sided confidence interval) [2] | | |
| Ratio difference between groups | 21.0% | (13.0%, 28.6%) |

| | |
|---|---|
| (95% two-sided confidence interval) [2] | |
| Unilateral P-value [3] | <0.0001 |

OC = actual observed value.
Percentages were calculated based on the number of evaluable subjects in each visit for each treatment group in the full analysis set. [symbol]
1] Binomial distribution based two-sided exact confidence intervals (Clopper-Pearson confidence intervals). [symbol]
2] Asymptotic confidence intervals for the differences in rates between groups obtained based on the normal distribution approximation method. [symbol]
3] Based on chi-square test or Fisher's Exact test. [symbol]
indicates data missing or illegible when filed

TABLE 26
Summary of mean change from baseline in VAS scores for dry eye symptoms
(full analysis set)

| | | |
|---|---|---|
| | Cyclosporine A | |
| | Ophthalmic Gel | Vehicle |
| FOR DRYNESS SENSE | (N = 315) | (N = 312) |

Baseline VAS score

| | | | | |
|---|---|---|---|---|
| Mean value (standard deviation) | 65.8 | (13.67) | 65.0 | (13.96) |

| | | |
|---|---|---|
| Median | 65.0 | 63.0 |
| Min-Max | 41-99 | 40-100 |

Visit 3 VAS score

| Number of cases | 314 | | 311 | |
|---|---|---|---|---|
| Mean value (standard deviation) | 53.9 | (21.34) | 52.5 | (22.42) |
| Median | 55.0 | | 53.0 | |
| Min-Max | 0-97 | | 3-97 | |
| Number of cases of change from baseline | 314 | | 311 | |
| Mean value (standard deviation) | −11.9 | (20.16) | −12.5 | (19.94) |

| | | |
|---|---|---|
| Median | −9.0 | −9.0 |
| Min-Max | 92-39 | −92-38 |
| ANOVA two-sided P-value | 0.7159 | |
| Kruskal Wallis rank sum test for two-sided p-value | 0.5904 | |

MMRM model [1]

| | | | | |
|---|---|---|---|---|
| Least squares mean (standard error) | −11.7 | (1.11) | −12.6 | (1.11) |

| 95% two-sided confidence interval | (−13.9, −9.6) | | (−14.8, −10.4) |
|---|---|---|---|
| Least squares mean (standard error) of the difference over | 0.9 | (1.57) | |

| | |
|---|---|
| the vehicle group | |
| 95% two-sided confidence interval | (−2.2, 4.0) |
| Bilateral P-value | 0.5804 |

Visit 4 VAS score

| Number of cases | 305 | | 303 | |
|---|---|---|---|---|
| Mean value (standard deviation) | 43.6 | (23.80) | 41.9 | (25.44) |
| Median | 44.0 | | 38.0 | |
| Min-Max | 0-98 | | 0-97 | |
| Number of cases of change from baseline | 305 | | 303 | |
| Mean value (standard deviation) | −22.2 | (23.53) | −23.0 | (23.88) |

| | | |
|---|---|---|
| Median | −20.0 | −23.0 |
| Min-Max | −93-32 | 98-39 |
| ANOVA two-sided P-value | 0.6951 | |
| Kruskal Wallis rank sum test for two-sided p-value | 0.6522 | |

MMRM model [1]

| | | | | |
|---|---|---|---|---|
| Least squares mean (standard error) | −22.1 | (1.32) | −23.1 | (1.32) |

| 95% two-sided confidence interval | (−24.6, −19.5) | | (−25.7, −20.5) |
|---|---|---|---|
| Least squares mean (standard error) of the difference over | 1.0 | (1.87) | |

| | |
|---|---|
| the vehicle group | |
| 95% two-sided confidence interval | (−2.6, 4.7) |
| Bilateral P-value | 0.5834 |

Visit 5 VAS score

| Number of cases | 298 | | 301 | |
|---|---|---|---|---|
| Mean value (standard deviation) | 36.7 | (25.13) | 33.7 | (24.32) |
| Median | 33.0 | | 28.0 | |
| Min-Max | 0-98 | | 0-99 | |
| Number of cases of change from baseline | 298 | | 301 | |
| Mean value (standard deviation) | −29.2 | (25.04) | −31.2 | (23.36) |

| | | |
|---|---|---|
| Median | −29.0 | −31.0 |
| Min-Max | 95-39 | −94-23 |
| ANOVA two-sided P-value | 0.3131 | |
| Kruskal Wallis rank sum test for two-sided p-value | 0.3456 | |

MMRM model [1]

| | | | | |
|---|---|---|---|---|
| Least squares mean (standard error) | −29.3 | (1.35) | 31.2 | (1.35) |

| 95% two-sided confidence interval | (−32.0, −26.7) | | (−33.8, −28.5) |
|---|---|---|---|
| Least squares mean (standard error) of the difference over | 1.8 | (1.91) | |

| the vehicle group | | |
|---|---|---|
| 95% two-sided confidence interval | (−1.9, 5.6) | |
| Bilateral P-value | 0.3427 | |
| | Cyclosporine A | |
| | Ophthalmic Gel | Vehicle |
| FOR BURNING/STINGING SENSATION | (N = 315) | (N = 312) |

Baseline VAS score

| Number of cases | 315 | | 312 | |
|---|---|---|---|---|
| Mean value (standard deviation) | 36.2 | (27.03) | 35.6 | (27.31) |

| | | |
|---|---|---|
| Median | 34.0 | 34.5 |
| Min-Max | 0-96 | 0-97 |

Visit 3 VAS score

| Number of cases | 314 | | 311 | |
|---|---|---|---|---|
| Mean value (standard deviation) | 37.4 | (26.72) | 33.1 | (27.60) |
| Median | 34.0 | | 28.0 | |
| Min-Max | 0-97 | | 0-97 | |
| Number of cases of change from baseline | 314 | | 311 | |
| Mean value (standard deviation) | 1.3 | (23.83) | −2.4 | (23.92) |

| | | |
|---|---|---|
| Median | 0.0 | −2.0 |
| Min-Max | 94-79 | −95-81 |
| ANOVA two-sided P-value | 0.0563 | |
| Kruskal Wallis rank sum test for two-sided p-value | 0.0110 | |

MMRM model [1]

| | | | | |
|---|---|---|---|---|
| Least squares mean (standard error) | 1.5 | (1.23) | −2.5 | (1.24) |

| 95% two-sided confidence interval | (−0.9, 3.9) | | (−5.0, −0.1) |
|---|---|---|---|
| Least squares mean (standard error) of the difference over | 4.0 | (1.74) | |

| | |
|---|---|
| the vehicle group | |
| 95% two-sided confidence interval | (0.6, 7.5) |
| Bilateral P-value | 0.0209 |

Visit 4 VAS score

| Number of cases | 305 | | 303 | |
|---|---|---|---|---|
| Mean value (standard deviation) | 31.6 | (26.06) | 25.1 | (24.18) |
| Median | 24.0 | | 17.0 | |
| Min-Max | 0-97 | | 0-96 | |
| Number of cases of change from baseline | 305 | | 303 | |
| Mean value (standard deviation) | −4.6 | (28.96) | −10.1 | (25.02) |

| | | |
|---|---|---|
| Median | −2.0 | −6.0 |
| Min-Max | −95-93 | −97-73 |
| ANOVA two-sided P-value | 0.0124 | |
| Kruskal Wallis rank sum test for two-sided p-value | 0.0093 | |

MMRM model [1]

| | | | | |
|---|---|---|---|---|
| Least squares mean (standard error) | −4.3 | (1.27) | −10.4 | (1.28) |

| 95% two-sided confidence interval | (−6.8, −1.8) | | (−12.9, −7.9) |
|---|---|---|---|
| Least squares mean (standard error) of the difference over | 6.1 | (1.80) | |

| | |
|---|---|
| the vehicle group | |
| 95% two-sided confidence interval | (2.6, 9.6) |
| Bilateral P-value | 0.0007 |

Visit 5 VAS score

| Number of cases | 298 | | 301 | |
|---|---|---|---|---|
| Mean value (standard deviation) | 27.4 | (24.52) | 19.8 | (21.07) |
| Median | 21.5 | | 12.0 | |
| Min-Max | 0-95 | | 0-95 | |
| Number of cases of change from baseline | 298 | | 301 | |
| Mean value (standard deviation) | −8.9 | (29.40) | −15.5 | (26.02) |

| | | |
|---|---|---|
| Median | −5.0 | −12.0 |
| Min-Max | 95-88 | 86-72 |
| ANOVA two-sided P-value | 0.0039 | |
| Kruskal Wallis rank sum test for two-sided p-value | 0.0015 | |

MMRM model [1]

| | | | | |
|---|---|---|---|---|
| Least squares mean (standard error) | −8.7 | (1.23) | −15.5 | (1.23) |

| 95% two-sided confidence interval | (−11.1, −6.3) | | (−17.9, −13.1) |
|---|---|---|---|
| Least squares mean (standard error) of the difference over | 6.8 | (1.74) | |

| the vehicle group | | |
|---|---|---|
| 95% two-sided confidence interval | (3.4, 10.2) | |
| Bilateral P-value | 0.0001 | |
| | Cyclosporine A | |
| | Ophthalmic Gel | Vehicle |
| FOR ITCHING | (N = 315) | (N = 312) |

Baseline VAS score

| Number of cases | 315 | | 312 | |
|---|---|---|---|---|
| Mean value (standard deviation) | 35.9 | (27.49) | 33.4 | (27.26) |

| | | |
|---|---|---|
| Median | 31.0 | 28.0 |
| Min-Max | 0-98 | 0-95 |

Visit 3 VAS score

| Number of cases | 314 | | 311 | |
|---|---|---|---|---|
| Mean value (standard deviation) | 33.5 | (26.04) | 30.3 | (26.31) |
| Median | 28.0 | | 23.0 | |
| Min-Max | 0-97 | | 0-94 | |
| Number of cases of change from baseline | 314 | | 311 | |
| Mean value (standard deviation) | −2.4 | (21.54) | −3.0 | (22.49) |

| | | |
|---|---|---|
| Median | −1.5 | −1.0 |
| Min-Max | −93-88 | 83-83 |
| ANOVA two-sided P-value | 0.7520 | |
| Kruskal Wallis rank sum test for two-sided p-value | 0.9336 | |

MMRM model [1]

| | | | | |
|---|---|---|---|---|
| Least squares mean (standard error) | −1.8 | (1.13) | −3.7 | (1.13) |

| 95% two-sided confidence interval | (−4.0, 0.4) | | (−5.9, −1.5) |
|---|---|---|---|
| Least squares mean (standard error) of the difference over | 1.9 | (1.60) | |

| | |
|---|---|
| the vehicle group | |
| 95% two-sided confidence interval | (−1.3, 5.0) |
| Bilateral P-value | 0.2444 |

Visit 4 VAS score

| Number of cases | 305 | | 303 | |
|---|---|---|---|---|
| Mean value (standard deviation) | 25.6 | (23, 64) | 24.6 | (23.76) |
| Median | 17.0 | | 14.0 | |
| Min-Max | 0-97 | | 0-94 | |
| Number of cases of change from baseline | 305 | | 303 | |
| Mean value (standard deviation) | −10.6 | (24.65) | −8.6 | (24.04) |

| | | |
|---|---|---|
| Median | −5.0 | −4.0 |
| Min-Max | 96-82 | −89-70 |
| ANOVA two-sided P-value | 0.3022 | |
| Kruskal Wallis rank sum test for two-sided p-value | 0.8539 | |

MMRM model [1]

| | | | | |
|---|---|---|---|---|
| Least squares mean (standard error) | −9.9 | (1.13) | −9.5 | (1.13) |

| 95% two-sided confidence interval | (−12.1, −7.7) | | (−11.7, −7.3) |
|---|---|---|---|
| Least squares mean (standard error) of the difference over | −0.4 | (1.60) | |

| | |
|---|---|
| the vehicle group | |
| 95% two-sided confidence interval | (−3.6, 2.7) |
| Bilateral P-value | 0.7873 |

Visit 5 VAS score

| Number of cases | 298 | | 301 | |
|---|---|---|---|---|
| Mean value (standard deviation) | 22.1 | (21.98) | 19.9 | (21.48) |
| Median | 14.0 | | 12.0 | |
| Min-Max | 0-91 | | 0-94 | |
| Number of cases of change from baseline | 298 | | 301 | |
| Mean value (standard deviation) | −14.5 | (27.13) | −13.4 | (24.82) |

| | | |
|---|---|---|
| Median | −7.5 | −9.0 |
| Min-Max | 96-77 | 94-72 |
| ANOVA two-sided P-value | 0.6243 | |
| Kruskal Wallis rank sum test for two-sided p-value | 0.9829 | |

MMRM model [1]

| | | | | |
|---|---|---|---|---|
| Least squares mean (standard error) | −13.6 | (1.14) | −14.1 | (1.13) |

| 95% two-sided confidence interval | (−15.8, −11.4) | | (−16.4, −11.9) |
|---|---|---|---|
| Least squares mean (standard error) of the difference over | 0.5 | (1.60) | |

| the vehicle group | | |
|---|---|---|
| 95% two-sided confidence interval | (−2.6, 3.7) | |
| Bilateral P-value | 0.7461 | |
| | Cyclosporine A | |
| | Ophthalmic Gel | Vehicle |
| FOR FOREIGN BODY SENSATION | (N = 315) | (N = 312) |

Baseline VAS score

| Number of cases | 315 | | 312 | |
|---|---|---|---|---|
| Mean value (standard deviation) | 43.8 | (27.91) | 41.1 | (28.55) |

| | | |
|---|---|---|
| Median | 44.0 | 41.0 |
| Min-Max | 0-99 | 0-98 |

Visit 3 VAS score

| Number of cases | 314 | | 311 | |
|---|---|---|---|---|
| Mean value (standard deviation) | 38.3 | (26.81) | 37.7 | (28.22) |
| Median | 39.0 | | 31.0 | |
| Min-Max | 0-98 | | 0-96 | |
| Number of cases of change from baseline | 314 | | 311 | |
| Mean value (standard deviation) | −5.5 | (22.00) | −3.4 | (25.27) |

| | | |
|---|---|---|
| Median | −2.0 | −3.0 |
| Min-Max | −90-63 | −80-83 |
| ANOVA two-sided P-value | 0.2872 | |
| Kruskal Wallis rank sum test for two-sided p-value | 0.9479 | |

MMRM model [1]

| | | | | |
|---|---|---|---|---|
| Least squares mean (standard error) | −4.8 | (1.21) | −4.0 | (1.21) |

| 95% two-sided confidence interval | (−7.1, −2.4) | | (−6.4, −1.7) |
|---|---|---|---|
| Least squares mean (standard error) of the difference over | −0.7 | (1.71) | |

| | |
|---|---|
| the vehicle group | |
| 95% two-sided confidence interval | (−4.1, 2.6) |
| Bilateral P-value | 0.6752 |

Visit 4 VAS score

| Number of cases | 305 | | 303 | |
|---|---|---|---|---|
| Mean value (standard deviation) | 32.2 | (25.68) | 29.9 | (26.17) |
| Median | 26.0 | | 24.0 | |
| Min-Max | 0-95 | | 0-96 | |
| Number of cases of change from baseline | 305 | | 303 | |
| Mean value (standard deviation) | −11.4 | (25.20) | −11.0 | (26.64) |

| | | |
|---|---|---|
| Median | −7.0 | −6.0 |
| Min-Max | 91-60 | −86-85 |
| ANOVA two-sided P-value | 0.8468 | |
| Kruskal Wallis rank sum test for two-sided p-value | 0.8816 | |

MMRM model [1]

| | | | | |
|---|---|---|---|---|
| Least squares mean (standard error) | −10.7 | (1.24) | −11.9 | (1.24) |

| 95% two-sided confidence interval | (−13.2, −8.3) | | (−14.3, −9.4) |
|---|---|---|---|
| Least squares mean (standard error) of the difference over | 1.1 | (1.75) | |

| | |
|---|---|
| the vehicle group | |
| 95% two-sided confidence interval | (−2.3, 4.6) |
| Bilateral P-value | 0.5202 |

Visit 5 VAS score

| Number of cases | 298 | | 301 | |
|---|---|---|---|---|
| Mean value (standard deviation) | 27.3 | (24.35) | 22.9 | (23.11) |
| Median | 20.5 | | 14.0 | |
| Min-Max | 0-96 | | 0-96 | |
| Number of cases of change from baseline | 298 | | 301 | |
| Mean value (standard deviation) | −16.6 | (27.07) | −17.9 | (28.63) |

| | | |
|---|---|---|
| Median | −14.0 | −15.0 |
| Min-Max | −89-65 | 91-71 |
| ANOVA two-sided P-value | 0.5516 | |
| Kruskal Wallis rank sum test for two-sided p-value | 0.4924 | |

MMRM model [1]

| | | | | |
|---|---|---|---|---|
| Least squares mean (standard error) | −15.8 | (1.25) | 18.6 | (1.25) |

| 95% two-sided confidence interval | (−18.3, −13.3) | | (−21.1, −16.1) |
|---|---|---|---|
| Least squares mean (standard error) of the difference over | 2.8 | (1.77) | |

| the vehicle group | | |
|---|---|---|
| 95% two-sided confidence interval | (−0.7, 6.3) | |
| Bilateral P-value | 0.1129 | |
| | Cyclosporine A | |
| | Ophthalmic Gel | Vehicle |
| FOR DISCOMFORT | (N = 315) | (N = 312) |

Baseline VAS score

| Number of cases | 315 | | 312 | |
|---|---|---|---|---|
| Mean value (standard deviation) | 49.7 | (26.61) | 50.5 | (27.16) |

| | | |
|---|---|---|
| Median | 52.0 | 54.0 |
| Min-Max | 0-98 | 0-96 |

Visit 3 VAS score

| Number of cases | 314 | | 311 | |
|---|---|---|---|---|
| Mean value (standard deviation) | 45.2 | (25.90) | 42.5 | (27.70) |
| Median | 47.5 | | 44.0 | |
| Min-Max | 0-99 | | 0-98 | |
| Number of cases of change from baseline | 314 | | 311 | |
| Mean value (standard deviation) | −4.5 | (25.04) | −7.9 | (26.40) |

| | | |
|---|---|---|
| Median | −2.0 | −5.0 |
| Min-Max | −86-93 | 95-76 |
| ANOVA two-sided P-value | 0.0946 | |
| Kruskal Wallis rank sum test for two-sided p-value | 0.0304 | |

MMRM model [1]

| | | | | |
|---|---|---|---|---|
| Least squares mean (standard error) | −4.6 | (1.28) | −7.7 | (1.28) |

| 95% two-sided confidence interval | (−7.2, −2.1) | | (−10.2, −5.2) |
|---|---|---|---|
| Least squares mean (standard error) of the difference over | 3.1 | (1.81) | |

| | |
|---|---|
| the vehicle group | |
| 95% two-sided confidence interval | (−0.5, 6.6) |
| Bilateral P-value | 0.0890 |

Visit 4 VAS score

| Number of cases | 305 | | 303 | |
|---|---|---|---|---|
| Mean value (standard deviation) | 37.1 | (26.67) | 33.9 | (26.55) |
| Median | 34.0 | | 29.0 | |
| Min-Max | 0-99 | | 0-97 | |
| Number of cases of change from baseline | 305 | | 303 | |
| Mean value (standard deviation) | −12.4 | (27.21) | −16.5 | (26.54) |

| | | |
|---|---|---|
| Median | −11.0 | −12.0 |
| Min-Max | −92-81 | 96-71 |
| ANOVA two-sided P-value | 0.0606 | |
| Kruskal Wallis rank sum test for two-sided p-value | 0.0646 | |

MMRM model [1]

| | | | | |
|---|---|---|---|---|
| Least squares mean (standard error) | −12.6 | (1.32) | −16.4 | (1.32) |

| 95% two-sided confidence interval | (−15.2, −10.0) | | (−19.0, −13.8) |
|---|---|---|---|
| Least squares mean (standard error) of the difference over | 3.8 | (1.87) | |

| | |
|---|---|
| the vehicle group | |
| 95% two-sided confidence interval | (0.1, 7.4) |
| Bilateral P-value | 0.0450 |

Visit 5 VAS score

| Number of cases | 298 | | 301 | |
|---|---|---|---|---|
| Mean value (standard deviation) | 30.9 | (26.01) | 26.8 | (25.56) |
| Median | 25.0 | | 17.0 | |
| Min-Max | 0-98 | | 0-98 | |
| Number of cases of change from baseline | 298 | | 301 | |
| Mean value (standard deviation) | −18.6 | (28.79) | −23.7 | (27.12) |

| | | |
|---|---|---|
| Median | −16.5 | −23.0 |
| Min-Max | −92-81 | −92-85 |
| ANOVA two-sided P-value | 0.0284 | |
| Kruskal Wallis rank sum test for two-sided p-value | 0.0158 | |

MMRM model [1]

| | | | | |
|---|---|---|---|---|
| Least squares mean (standard error) | −18.9 | (1.34) | −23.3 | (1.34) |

| 95% two-sided confidence interval | (−21.6, −16.3) | | (−26.0, −20.7) |
|---|---|---|---|
| Least squares mean (standard error) of the difference over | 4.4 | (1.90) | |

| the vehicle group | | |
|---|---|---|
| 95% two-sided confidence interval | (0.7, 8.2) | |
| Bilateral P-value | 0.0201 | |
| | Cyclosporine A | |
| | Ophthalmic Gel | Vehicle |
| FOR PHOTOPHOBIA | (N = 315) | (N = 312) |

Baseline VAS score

| Number of cases | 315 | | 312 | |
|---|---|---|---|---|
| Mean value (standard deviation) | 43.8 | (28.07) | 40.7 | (29.84) |

| | | |
|---|---|---|
| Median | 44.0 | 41.5 |
| Min-Max | 0-99 | 0-100 |

Visit 3 VAS score

| Number of cases | 314 | | 311 | |
|---|---|---|---|---|
| Mean value (standard deviation) | 40.6 | (27.40) | 35.5 | (29.66) |
| Median | 41.0 | | 31.0 | |
| Min-Max | 0-99 | | 0-100 | |
| Number of cases of change from baseline | 314 | | 311 | |
| Mean value (standard deviation) | −3.2 | (21.98) | −5.2 | (23.29) |

| | | |
|---|---|---|
| Median | 0.0 | −3.0 |
| Min-Max | −97-58 | 81-79 |
| ANOVA two-sided P-value | 0.2641 | |
| Kruskal Wallis rank sum test for two-sided p-value | 0.0178 | |

MMRM model [1]

| | | | | |
|---|---|---|---|---|
| Least squares mean (standard error) | −2.7 | (1.17) | −5.9 | (1.18) |

| 95% two-sided confidence interval | (−5.0, −0.4) | | (−8.2, −3.6) |
|---|---|---|---|
| Least squares mean (standard error) of the difference over | 3.2 | (1.66) | |

| | |
|---|---|
| the vehicle group | |
| 95% two-sided confidence interval | (−0.1, 6.4) |
| Bilateral P-value | 0.0559 |

Visit 4 VAS score

| Number of cases | 305 | | 303 | |
|---|---|---|---|---|
| Mean value (standard deviation) | 34.7 | (26.85) | 30.7 | (28.96) |
| Median | 31.0 | | 24.0 | |
| Min-Max | 0-100 | | 0-100 | |
| Number of cases of change from baseline | 305 | | 303 | |
| Mean value (standard deviation) | −9.3 | (25.28) | −10.0 | (23.64) |

| | | |
|---|---|---|
| Median | −7.0 | −6.0 |
| Min-Max | −98-84 | −85-85 |
| ANOVA two-sided P-value | 0.7350 | |
| Kruskal Wallis rank sum test for two-sided p-value | 0.6992 | |

MMRM model [1]

| | | | | |
|---|---|---|---|---|
| Least squares mean (standard error) | −8.6 | (1.24) | −10.5 | (1.24) |

| 95% two-sided confidence interval | (−11.0, −6.1) | | (−13.0, −8.1) |
|---|---|---|---|
| Least squares mean (standard error) of the difference over | 2.0 | (1.76) | |

| | |
|---|---|
| the vehicle group | |
| 95% two-sided confidence interval | (−1.5, 5.4) |
| Bilateral P-value | 0.2658 |

Visit 5 VAS score

| Number of cases | 298 | | 301 | |
|---|---|---|---|---|
| Mean value (standard deviation) | 30.9 | (26.09) | 25.3 | (27.89) |
| Median | 24.5 | | 15.0 | |
| Min-Max | 0-98 | | 0-100 | |
| Number of cases of change from baseline | 298 | | 301 | |
| Mean value (standard deviation) | −13.2 | (26.01) | −15.7 | (24.96) |

| | | |
|---|---|---|
| Median | −10.0 | −13.0 |
| Min-Max | −98-63 | 77-74 |
| ANOVA two-sided P-value | 0.2368 | |
| Kruskal Wallis rank sum test for two-sided p-value | 0.0954 | |

MMRM model [1]

| | | | | |
|---|---|---|---|---|
| Least squares mean (standard error) | −12.5 | (1.27) | −15.9 | (1.27) |

| 95% two-sided confidence interval | (−15.0, −10.0) | | (−18.4, −13.4) |
|---|---|---|---|
| Least squares mean (standard error) of the difference over | 3.4 | (1.80) | |

| the vehicle group | | |
|---|---|---|
| 95% two-sided confidence interval | (−0.1, 6.9) | |
| Bilateral P-value | 0.0593 | |
| | Cyclosporine A | |
| | Ophthalmic Gel | Vehicle |
| FOR PAIN | (N = 315) | (N = 312) |

Baseline VAS score

| Number of cases | 315 | | 312 | |
|---|---|---|---|---|
| Mean value (standard deviation) | 27.9 | (25.65) | 26.0 | (25.90) |

| | | |
|---|---|---|
| Median | 19.0 | 16.5 |
| Min-Max | 0-98 | 0-98 |

Visit 3 VAS score

| Number of cases | 314 | | 311 | |
|---|---|---|---|---|
| Mean value (standard deviation) | 28.5 | (25.55) | 23.1 | (25.74) |
| Median | 21.5 | | 11.0 | |
| Min-Max | 0-98 | | 0-98 | |
| Number of cases of change from baseline | 314 | | 311 | |
| Mean value (standard deviation) | 0.7 | (22.84) | −2.8 | (21.56) |

| | | |
|---|---|---|
| Median | 0.0 | 0.0 |
| Min-Max | 96-84 | −82-80 |
| ANOVA two-sided P-value | 0.0472 | |
| Kruskal Wallis rank sum test for two-sided p-value | 0.0206 | |

MMRM model [1]

| | | | | |
|---|---|---|---|---|
| Least squares mean (standard error) | 1.1 | (1.14) | −3.3 | (1.14) |

| 95% two-sided confidence interval | (−1.1, 3.3) | | (−5.6, −1.1) |
|---|---|---|---|
| Least squares mean (standard error) of the difference over | 4.4 | (1.61) | |

| | |
|---|---|
| the vehicle group | |
| 95% two-sided confidence interval | (1.3, 7.6) |
| Bilateral P-value | 0.0063 |

Visit 4 VAS score

| Number of cases | 305 | | 303 | |
|---|---|---|---|---|
| Mean value (standard deviation) | 24.3 | (24.19) | 18.8 | (23.09) |
| Median | 14.0 | | 8.0 | |
| Min-Max | 0-100 | | 0-100 | |
| Number of cases of change from baseline | 305 | | 303 | |
| Mean value (standard deviation) | −3.8 | (24.38) | −7.1 | (20.75) |

| | | |
|---|---|---|
| Median | −2.0 | −2.0 |
| Min-Max | −97-90 | 76-61 |
| ANOVA two-sided P-value | 0.0650 | |
| Kruskal Wallis rank sum test for two-sided p-value | 0.0892 | |

MMRM model [1]

| | | | | |
|---|---|---|---|---|
| Least squares mean (standard error) | −3.2 | (1.10) | −7.6 | (1.10) |

| 95% two-sided confidence interval | (−5.4, −1.1) | | (−9.8, −5.5) |
|---|---|---|---|
| Least squares mean (standard error) of the difference over | 4.4 | (1.55) | |

| | |
|---|---|
| the vehicle group | |
| 95% two-sided confidence interval | (1.3, 7.4) |
| Bilateral P-value | 0.0049 |

Visit 5 VAS score

| Number of cases | 298 | | 301 | |
|---|---|---|---|---|
| Mean value (standard deviation) | 21.9 | (23.42) | 14.1 | (20.10) |
| Median | 13.0 | | 5.0 | |
| Min-Max | 0-98 | | 0-100 | |
| Number of cases of change from baseline | 298 | | 301 | |
| Mean value (standard deviation) | −6.2 | (25.60) | −11.9 | (22.67) |

| | | |
|---|---|---|
| Median | −3.0 | −5.0 |
| Min-Max | −98-89 | −88-53 |
| ANOVA two-sided P-value | 0.0041 | |
| Kruskal Wallis rank sum test for two-sided p-value | 0.0102 | |

MMRM model [1]

| | | | | |
|---|---|---|---|---|
| Least squares mean (standard error) | −5.8 | (1.11) | −12.2 | (1.11) |

| 95% two-sided confidence interval | (−8.0, −3.7) | | (−14.3, −10.0) |
|---|---|---|---|
| Least squares mean (standard error) of the difference over | 6.3 | (1.56) | |

| | |
|---|---|
| the vehicle group | |
| 95% two-sided confidence interval | (3.3, 9.4) |
| Bilateral P-value | <0.0001 |

MMRM = repeated measures mixed effects model.
Baseline was defined as the last non-missing data before the start of treatment with the trial drug.
[1] The actual observed change in VAS scores relative to baseline at each post-baseline visit was used as the dependent variable, treatment group, visit, and treatment group-visit interaction were the independent variables, and baseline VAS scores were used as covariates with an unstructured within-subject variance-covariance structure (UN),

TABLE 27
Summary of mean change from baseline in VAS scores for dry eye symptoms
(consistent with the protocol set)

| | | |
|---|---|---|
| | Cyclosporine A | |
| | Ophthalmic Gel | Vehicle |
| FOR DRYNESS SENSE | (N = 287) | (N = 295) |

Baseline VAS score

| Number of cases | 287 | | 295 | |
|---|---|---|---|---|
| Mean value (standard deviation) | 66.0 | (13.89) | 65.1 | (13.99) |

| | | |
|---|---|---|
| Median | 65.0 | 64.0 |
| Min-Max | 41-99 | 40-100 |

Visit 3 VAS score

| Number of cases | 287 | | 295 | |
|---|---|---|---|---|
| Mean value (standard deviation) | 54.6 | (21.11) | 52.2 | (22.57) |
| Median | 55.0 | | 53.0 | |
| Min-Max | 1-97 | | 3-97 | |
| Number of cases of change from baseline | 287 | | 295 | |
| Mean value (standard deviation) | −11.5 | (20.21) | −12.9 | (20.24) |

| | | |
|---|---|---|
| Median | −8.0 | −9.0 |
| Min-Max | 92-39 | −92-38 |
| ANOVA two-sided P-value | 0.3984 | |
| Kruskal Wallis rank sum test for two-sided p-value | 0.3028 | |

MMRM model [1]

| | | | | |
|---|---|---|---|---|
| Least squares mean (standard error) | −11.3 | (1.17) | −13.1 | (1.15) |

| 95% two-sided confidence interval | (−13.6, −9.0) | | (−15.3, −10.8) |
|---|---|---|---|
| Least squares mean (standard error) of the difference over | 1.8 | (1.64) | |

| | |
|---|---|
| the vehicle group | |
| 95% two-sided confidence interval | (−1.5, 5.0) |
| Bilateral P-value | 0.2837 |

Visit 4 VAS score

| Number of cases | 287 | | 294 | |
|---|---|---|---|---|
| Mean value (standard deviation) | 44.1 | (23.61) | 41.6 | (25.32) |
| Median | 44.0 | | 38.0 | |
| Min-Max | 1-98 | | 0-97 | |
| Number of cases of change from baseline | 287 | | 294 | |
| Mean value (standard deviation) | −22.0 | (23.48) | −23.4 | (23.86) |

| | | |
|---|---|---|
| Median | −20.0 | −24.0 |
| Min-Max | −93-32 | 98-39 |
| ANOVA two-sided P-value | 0.4822 | |
| Kruskal Wallis rank sum test for two-sided p-value | 0.4426 | |

MMRM model [1]

| | | | | |
|---|---|---|---|---|
| Least squares mean (standard error) | −21.8 | (1.36) | −23.6 | (1.34) |

| 95% two-sided confidence interval | (−24.5, −19.1) | | (−26.2, −20.9) |
|---|---|---|---|
| Least squares mean (standard error) of the difference over | 1.8 | (1.91) | |

| | |
|---|---|
| the vehicle group | |
| 95% two-sided confidence interval | (−2.0, 5.5) |
| Bilateral P-value | 0.3555 |

Visit 5 VAS score

| Number of cases | 287 | | 295 | |
|---|---|---|---|---|
| Mean value (standard deviation) | 37.0 | (25.10) | 33.9 | (24.43) |
| Median | 34.0 | | 29.0 | |
| Min-Max | 0-98 | | 0-99 | |
| Number of cases of change from baseline | 287 | | 295 | |
| Mean value (standard deviation) | −29.1 | (25.17) | −31.2 | (23.51) |

| | | |
|---|---|---|
| Median | −29.0 | −31.0 |
| Min-Max | 95-39 | −94-23 |
| ANOVA two-sided P-value | 0.2986 | |
| Kruskal Wallis rank sum test for two-sided p-value | 0.3251 | |

MMRM model [1]

| | | | | |
|---|---|---|---|---|
| Least squares mean (standard error) | −28.9 | (1.39) | −31.3 | (1.37) |

| 95% two-sided confidence interval | (−31.6, −26.2) | | (−34.0, −28.6) |
|---|---|---|---|
| Least squares mean (standard error) of the difference over | 2.4 | (1.95) | |

| the vehicle group | | |
|---|---|---|
| 95% two-sided confidence interval | (−1.4. 6.3) | |
| Bilateral P-value | 0.2125 | |
| | Cyclosporine A | |
| | Ophthalmic Gel | Vehicle |
| FOR BURNING/STINGING SENSATION | (N = 287) | (N = 295) |

Baseline VAS score

| Number of cases | 287 | | 295 | |
|---|---|---|---|---|
| Mean value (standard deviation) | 36.5 | (27.27) | 35.1 | (27.08) |

| | | |
|---|---|---|
| Median | 34.0 | 34.0 |
| Min-Max | 0-96 | 0-97 |

Visit 3 VAS score

| Number of cases | 287 | | 295 | |
|---|---|---|---|---|
| Mean value (standard deviation) | 37.7 | (26.73) | 32.7 | (27.37) |
| Median | 34.0 | | 27.0 | |
| Min-Max | 0-97 | | 0-96 | |
| Number of cases of change from baseline | 287 | | 295 | |
| Mean value (standard deviation) | 1.2 | (23.75) | −2.3 | (24.01) |

| | | |
|---|---|---|
| Median | 0.0 | −2.0 |
| Min-Max | 94-79 | −95-81 |
| ANOVA two-sided P-value | 0.0771 | |
| Kruskal Wallis rank sum test for two-sided p-value | 0.0120 | |

MMRM model [1]

| | | | | |
|---|---|---|---|---|
| Least squares mean (standard error) | 1.6 | (1.29) | −2.7 | (1.27) |

| 95% two-sided confidence interval | (−0.9, 4.1) | | (−5.2, −0.2) |
|---|---|---|---|
| Least squares mean (standard error) of the difference over | 4.3 | (1.81) | |

| | |
|---|---|
| the vehicle group | |
| 95% two-sided confidence interval | (0.8, 7.9) |
| Bilateral P-value | 0.0174 |

Visit 4 VAS score

| Number of cases | 287 | | 294 | |
|---|---|---|---|---|
| Mean value (standard deviation) | 31.9 | (26.00) | 24.7 | (23.81) |
| Median | 24.0 | | 17.0 | |
| Min-Max | 0-97 | | 0-95 | |
| Number of cases of change from baseline | 287 | | 294 | |
| Mean value (standard deviation) | −4.7 | (28.64) | −10.2 | (25.08) |

| | | |
|---|---|---|
| Median | −2.0 | −6.0 |
| Min-Max | −95-93 | −97-73 |
| ANOVA two-sided P-value | 0.0134 | |
| Kruskal Wallis rank sum test for two-sided p-value | 0.0067 | |

MMRM model [1]

| | | | | |
|---|---|---|---|---|
| Least squares mean (standard error) | −4.2 | (1.30) | −10.7 | (1.28) |

| 95% two-sided confidence interval | (−6.8, −1.7) | | (−13.3, −8.2) |
|---|---|---|---|
| Least squares mean (standard error) of the difference over | 6.5 | (1.83) | |

| | |
|---|---|
| the vehicle group | |
| 95% two-sided confidence interval | (2.9, 10.1) |
| Bilateral P-value | 0.0004 |

Visit 5 VAS score

| Number of cases | 287 | | 295 | |
|---|---|---|---|---|
| Mean value (standard deviation) | 27.5 | (24.61) | 19.8 | (21.01) |
| Median | 21.0 | | 12.0 | |
| Min-Max | 0-95 | | 0-95 | |
| Number of cases of change from baseline | 287 | | 295 | |
| Mean value (standard deviation) | −9.0 | (29.73) | −15.2 | (25.97) |

| | | |
|---|---|---|
| Median | −5.0 | −12.0 |
| Min-Max | 95-88 | 86-72 |
| ANOVA two-sided P-value | 0.0076 | |
| Kruskal Wallis rank sum test for two-sided p-value | 0.0034 | |

MMRM model [1]

| | | | | |
|---|---|---|---|---|
| Least squares mean (standard error) | −8.6 | (1.26) | −15.6 | (1.25) |

| 95% two-sided confidence interval | (−11.1, −6.1) | | (−18.0, −13.1) |
|---|---|---|---|
| Least squares mean (standard error) of the difference over | 7.0 | (1.77) | |

| the vehicle group | | |
|---|---|---|
| 95% two-sided confidence interval | (3.5, 10.5) | |
| Bilateral P-value | <0.0001 | |
| | Cyclosporine A | |
| | Ophthalmic Gel | Vehicle |
| FOR ITCHING | (N = 287) | (N = 295) |

Baseline VAS score

| Number of cases | 287 | | 295 | |
|---|---|---|---|---|
| Mean value (standard deviation) | 36.5 | (27.64) | 33.1 | (26.99) |

| | | |
|---|---|---|
| Median | 32.0 | 28.0 |
| Min-Max | 0-98 | 0-95 |

Visit 3 VAS score

| Number of cases | 287 | | 295 | |
|---|---|---|---|---|
| Mean value (standard deviation) | 34.0 | (26.32) | 30.6 | (26.44) |
| Median | 29.0 | | 23.0 | |
| Min-Max | 0-97 | | 0-94 | |
| Number of cases of change from baseline | 287 | | 295 | |
| Mean value (standard deviation) | −2.6 | (21.81) | −2.5 | (22.35) |

| | | |
|---|---|---|
| Median | −2.0 | −1.0 |
| Min-Max | −93-88 | 83-83 |
| ANOVA two-sided P-value | 0.9844 | |
| Kruskal Wallis rank sum test for two-sided p-value | 0.8763 | |

MMRM model [1]

| | | | | |
|---|---|---|---|---|
| Least squares mean (standard error) | −1.7 | (1.19) | −3.4 | (1.18) |

| 95% two-sided confidence interval | (−4.0.0.7) | | (−5.7, −1.1) |
|---|---|---|---|
| Least squares mean (standard error) of the difference over | 1.7 | (1.67) | |

| | |
|---|---|
| the vehicle group | |
| 95% two-sided confidence interval | (−1.6, 5.0) |
| Bilateral P-value | 0.3173 |

Visit 4 VAS score

| Number of cases | 287 | | 294 | |
|---|---|---|---|---|
| Mean value (standard deviation) | 25.8 | (23.84) | 24.4 | (23.66) |
| Median | 16.0 | | 14.0 | |
| Min-Max | 0-97 | | 0-94 | |
| Number of cases of change from baseline | 287 | | 294 | |
| Mean value (standard deviation) | −10.7 | (24.83) | −8.6 | (24.15) |

| | | |
|---|---|---|
| Median | −5.0 | −4.0 |
| Min-Max | 96-82 | −89-70 |
| ANOVA two-sided P-value | 0.2883 | |
| Kruskal Wallis rank sum test for two-sided p-value | 0.8531 | |

MMRM model [1]

| | | | | |
|---|---|---|---|---|
| Least squares mean (standard error) | −9.8 | (1.18) | −9.5 | (1.16) |

| 95% two-sided confidence interval | (−12.1, −7.5) | | (−11.7, −7.2) |
|---|---|---|---|
| Least squares mean (standard error) of the difference over | −0.4 | (1.65) | |

| | |
|---|---|
| the vehicle group | |
| 95% two-sided confidence interval | (−3.6. 2.9) |
| Bilateral P-value | 0.8215 |

Visit 5 VAS score

| Number of cases | 287 | | 295 | |
|---|---|---|---|---|
| Mean value (standard deviation) | 22.2 | (22.10) | 20.0 | (21.45) |
| Median | 14.0 | | 12.0 | |
| Min-Max | 0-91 | | 0-94 | |
| Number of cases of change from baseline | 287 | | 295 | |
| Mean value (standard deviation) | −14.4 | (27.35) | −13.2 | (24.90) |

| | | |
|---|---|---|
| Median | −7.0 | −9.0 |
| Min-Max | 96-77 | 94-72 |
| ANOVA two-sided P-value | 0.5898 | |
| Kruskal Wallis rank sum test for two-sided p-value | 0.9567 | |

MMRM model [1]

| | | | | |
|---|---|---|---|---|
| Least squares mean (standard error) | −13.5 | (1.17) | −14.0 | (1.15) |

| 95% two-sided confidence interval | (−15.8, −11.2) | | (−16.3, −11.7) |
|---|---|---|---|
| Least squares mean (standard error) of the difference over | 0.5 | (1.64) | |

| the vehicle group | | |
|---|---|---|
| 95% two-sided confidence interval | (−2.7, 3.8) | |
| Bilateral P-value | 0.7410 | |
| | Cyclosporine A | |
| | Ophthalmic Gel | Vehicle |
| FOR FOREIGN BODY SENSATION | (N = 287) | (N = 295) |

Baseline VAS score

| Number of cases | 287 | | 295 | |
|---|---|---|---|---|
| Mean value (standard deviation) | 44.1 | (28.06) | 40.7 | (28.57) |

| | | |
|---|---|---|
| Median | 45.0 | 41.0 |
| Min-Max | 0-96 | 0-98 |

Visit 3 VAS score

| Number of cases | 287 | | 295 | |
|---|---|---|---|---|
| Mean value (standard deviation) | 38.4 | (27.16) | 37.6 | (28.08) |
| Median | 39.0 | | 31.0 | |
| Min-Max | 0-98 | | 0-96 | |
| Number of cases of change from baseline | 287 | | 295 | |
| Mean value (standard deviation) | −5.7 | (22.26) | −3.0 | (25.12) |

| | | |
|---|---|---|
| Median | −2.0 | −3.0 |
| Min-Max | −90-63 | 80-83 |
| ANOVA two-sided P-value | 0.1762 | |
| Kruskal Wallis rank sum test for two-sided p-value | 0.8245 | |

MMRM model [1]

| | | | | |
|---|---|---|---|---|
| Least squares mean (standard error) | −4.9 | (1.27) | −3.9 | (1.25) |

| 95% two-sided confidence interval | (−7.3, −2.4) | | (−6.3.−1.4) |
|---|---|---|---|
| Least squares mean (standard error) of the difference over | −1.0 | (1.79) | |

| | |
|---|---|
| the vehicle group | |
| 95% two-sided confidence interval | (−4.5, 2.5) |
| Bilateral P-value | 0.5854 |

Visit 4 VAS score

| Number of cases | 287 | | 294 | |
|---|---|---|---|---|
| Mean value (standard deviation) | 32.4 | (25.65) | 29.4 | (25.76) |
| Median | 26.0 | | 24.0 | |
| Min-Max | 0-93 | | 0-96 | |
| Number of cases of change from baseline | 287 | | 294 | |
| Mean value (standard deviation) | −11.7 | (25.02) | −11.2 | (26.63) |

| | | |
|---|---|---|
| Median | −8.0 | −6.0 |
| Min-Max | 91-60 | −86-85 |
| ANOVA two-sided P-value | 0.8207 | |
| Kruskal Wallis rank sum test for two-sided p-value | 0.8749 | |

MMRM model [1]

| | | | | |
|---|---|---|---|---|
| Least squares mean (standard error) | −10.8 | (1.27) | −12.1 | (1.26) |

| 95% two-sided confidence interval | (−13.3, −8.3) | | (−14.6, −9.7) |
|---|---|---|---|
| Least squares mean (standard error) of the difference over | 1.3 | (1.79) | |

| | |
|---|---|
| the vehicle group | |
| 95% two-sided confidence interval | (−2.2, 4.8) |
| Bilateral P-value | 0.4774 |

Visit 5 VAS score

| Number of cases | 287 | | 295 | |
|---|---|---|---|---|
| Mean value (standard deviation) | 27.4 | (24.48) | 22.9 | (23.10) |
| Median | 21.0 | | 14.0 | |
| Min-Max | 0-96 | | 0-96 | |
| Number of cases of change from baseline | 287 | | 295 | |
| Mean value (standard deviation) | −16.7 | (26.99) | −17.8 | (28.37) |

| | | |
|---|---|---|
| Median | −14.0 | −15.0 |
| Min-Max | −89-65 | 91-71 |
| ANOVA two-sided P-value | 0.6237 | |
| Kruskal Wallis rank sum test for two-sided p-value | 0.5379 | |

MMRM model [1]

| | | | | |
|---|---|---|---|---|
| Least squares mean (standard error) | −15.8 | (1.28) | −18.6 | (1.26) |

| 95% two-sided confidence interval | (−18.3, −13.3) | | (−21.1, −16.2) |
|---|---|---|---|
| Least squares mean (standard error) of the difference over | 2.8 | (1.79) | |

| the vehicle group | | |
|---|---|---|
| 95% two-sided confidence interval | (−0.7, 6.3) | |
| Bilateral P-value | 0.1167 | |
| | Cyclosporine A | |
| | Ophthalmic Gel | Vehicle |
| FOR DISCOMFORT | (N = 287) | (N = 295) |

Baseline VAS score

| Number of cases | 287 | | 295 | |
|---|---|---|---|---|
| Mean value (standard deviation) | 49.3 | (26.85) | 50.2 | (27.36) |

| | | |
|---|---|---|
| Median | 51.0 | 53.0 |
| Min-Max | 0-98 | 0-96 |

Visit 3 VAS score

| Number of cases | 287 | | 295 | |
|---|---|---|---|---|
| Mean value (standard deviation) | 45.0 | (26.19) | 42.0 | (27.51) |
| Median | 47.0 | | 44.0 | |
| Min-Max | 0-99 | | 0-98 | |
| Number of cases of change from baseline | 287 | | 295 | |
| Mean value (standard deviation) | −4.3 | (24.58) | −8.2 | (26.28) |

| | | |
|---|---|---|
| Median | −1.0 | −6.0 |
| Min-Max | −86-93 | 95-76 |
| ANOVA two-sided P-value | 0.0635 | |
| Kruskal Wallis rank sum test for two-sided p-value | 0.0136 | |

MMRM model [1]

| | | | | |
|---|---|---|---|---|
| Least squares mean (standard error) | −4.5 | (1.32) | −8.0 | (1.30) |

| 95% two-sided confidence interval | (−7.1, −1.9) | | (−10.5, −5.4) |
|---|---|---|---|
| Least squares mean (standard error) of the difference over | 3.4 | (1.86) | |

| | |
|---|---|
| the vehicle group | |
| 95% two-sided confidence interval | (−0.2, 7.1) |
| Bilateral P-value | 0.0655 |

Visit 4 VAS score

| Number of cases | 287 | | 294 | |
|---|---|---|---|---|
| Mean value (standard deviation) | 36.9 | (26.56) | 33.3 | (26.31) |
| Median | 34.0 | | 27.5 | |
| Min-Max | 0-99 | | 0-97 | |
| Number of cases of change from baseline | 287 | | 294 | |
| Mean value (standard deviation) | −12.4 | (26.70) | −16.8 | (26.67) |

| | | |
|---|---|---|
| Median | −10.0 | −12.0 |
| Min-Max | −91-81 | 96-71 |
| ANOVA two-sided P-value | 0.0452 | |
| Kruskal Wallis rank sum test for two-sided p-value | 0.0407 | |

MMRM model [1]

| | | | | |
|---|---|---|---|---|
| Least squares mean (standard error) | −12.6 | (1.35) | −16.6 | (1.33) |

| 95% two-sided confidence interval | (−15.2, −9.9) | | (−19.2, −14.0) |
|---|---|---|---|
| Least squares mean (standard error) of the difference over | 4.0 | (1.90) | |

| | |
|---|---|
| the vehicle group | |
| 95% two-sided confidence interval | (0.3, 7.8) |
| Bilateral P-value | 0.0338 |

Visit 5 VAS score

| Number of cases | 287 | | 295 | |
|---|---|---|---|---|
| Mean value (standard deviation) | 31.1 | (26.12) | 27.0 | (25, 62) |
| Median | 25.0 | | 17.0 | |
| Min-Max | 0-98 | | 0-98 | |
| Number of cases of change from baseline | 287 | | 295 | |
| Mean value (standard deviation) | −18.1 | (28.62) | −23.3 | (26.95) |

| | | |
|---|---|---|
| Median | −16.0 | −23.0 |
| Min-Max | −92-81 | −92-85 |
| ANOVA two-sided P-value | 0.0269 | |
| Kruskal Wallis rank sum test for two-sided p-value | 0.0128 | |

MMRM mode [1]

| | | | | |
|---|---|---|---|---|
| Least squares mean (standard error) | −18.4 | (1.37) | −23.0 | (1.35) |

| 95% two-sided confidence interval | (−21.1, −15.7) | | (−25.7, −20.4) |
|---|---|---|---|
| Least squares mean (standard error) of the difference over | 4.6 | (1.92) | |

| the vehicle group | | |
|---|---|---|
| 95% two-sided confidence interval | (0.8, 8.4) | |
| Bilateral P-value | 0.0166 | |
| | Cyclosporine A | |
| | Ophthalmic Gel | Vehicle |
| FOR PHOTOPHOBIA | (N = 287) | (N = 295) |

Baseline VAS score

| Number of cases | 287 | | 295 | |
|---|---|---|---|---|
| Mean value (standard deviation) | 44.1 | (27.89) | 40.8 | (29.66) |

| | | |
|---|---|---|
| Median | 44.0 | 42.0 |
| Min-Max | 0-99 | 0-100 |

Visit 3 VAS score

| Number of cases | 287 | | 295 | |
|---|---|---|---|---|
| Mean value (standard deviation) | 40.7 | (27.26) | 35.4 | (29.57) |
| Median | 41.0 | | 31.0 | |
| Min-Max | 0-99 | | 0-100 | |
| Number of cases of change from baseline | 287 | | 295 | |
| Mean value (standard deviation) | −3.4 | (22.07) | −5.3 | (23.17) |

| | | |
|---|---|---|
| Median | 0.0 | −3.0 |
| Min-Max | 97-58 | 81-79 |
| ANOVA two-sided P-value | 0.2927 | |
| Kruskal Wallis rank sum test for two-sided p-value | 0.0224 | |

MMRM model [1]

| | | | | |
|---|---|---|---|---|
| Least squares mean (standard error) | −2.7 | (1.23) | −6.0 | (1.21) |

| 95% two-sided confidence interval | (−5.1, −0.3) | | (−8.3, −3.6) |
|---|---|---|---|
| Least squares mean (standard error) of the difference over | 3.2 | (1.72) | |

| | |
|---|---|
| the vehicle group | |
| 95% two-sided confidence interval | (−0.1, 6.6) |
| Bilateral P-value | 0.0608 |

Visit 4 VAS score

| Number of cases | 287 | | 294 | |
|---|---|---|---|---|
| Mean value (standard deviation) | 34.5 | (26.33) | 30.4 | (28, 61) |
| Median | 31.0 | | 24.0 | |
| Min-Max | 0-100 | | 0-100 | |
| Number of cases of change from baseline | 287 | | 294 | |
| Mean value (standard deviation) | −9.6 | (24.90) | −10.2 | (23.06) |

| | | |
|---|---|---|
| Median | −7.0 | −6.0 |
| Min-Max | −98-84 | 85-63 |
| ANOVA two-sided P-value | 0.7837 | |
| Kruskal Wallis rank sum test for two-sided p-value | 0.7329 | |

MMRM model [1]

| | | | | |
|---|---|---|---|---|
| Least squares mean (standard error) | −9.0 | (1.25) | −10.8 | (1.24) |

| 95% two-sided confidence interval | (−11.4, −6.5) | | (−13.2, −8.3) |
|---|---|---|---|
| Least squares mean (standard error) of the difference over | 1.8 | (1.76) | |

| | |
|---|---|
| the vehicle group | |
| 95% two-sided confidence interval | (−1.6, 5.3) |
| Bilateral P-value | 0.3024 |

Visit 5 VAS score

| Number of cases | 287 | | 295 | |
|---|---|---|---|---|
| Mean value (standard deviation) | 30.8 | (26.11) | 25.5 | (27.96) |
| Median | 25.0 | | 15.0 | |
| Min-Max | 0-98 | | 0-100 | |
| Number of cases of change from baseline | 287 | | 295 | |
| Mean value (standard deviation) | −13.3 | (26.27) | −15.3 | (24.94) |

| | | |
|---|---|---|
| Median | −10.0 | −13.0 |
| Min-Max | −98-63 | 77-74 |
| ANOVA two-sided P-value | 0.3328 | |
| Kruskal Wallis rank sum test for two-sided p-value | 0.1435 | |

MMRM model [1]

| | | | | |
|---|---|---|---|---|
| Least squares mean (standard error) | −12.6 | (1.31) | −15.9 | (1.29) |

| 95% two-sided confidence interval | (−15.2, −10.0) | | (−18.5, −13.4) |
|---|---|---|---|
| Least squares mean (standard error) of the difference over | 3.3 | (1.84) | |

| the vehicle group | | |
|---|---|---|
| 95% two-sided confidence interval | (−0.3, 6.9) | |
| Bilateral P-value | 0.0716 | |
| | Cyclosporine A | |
| | Ophthalmic Gel | Vehicle |
| FOR PAIN | (N = 287) | (N = 295) |

Baseline VAS score

| Number of cases | 287 | | 295 | |
|---|---|---|---|---|
| Mean value (standard deviation) | 28.3 | (26.04) | 25.7 | (25.74) |

| | | |
|---|---|---|
| Median | 20.0 | 16.0 |
| Min-Max | 0-98 | 0-98 |

Visit 3 VAS score

| Number of cases | 287 | | 295 | |
|---|---|---|---|---|
| Mean value (standard deviation) | 29.0 | (25.87) | 22.8 | (25.79) |
| Median | 22.0 | | 10.0 | |
| Min-Max | 0-98 | | 0-98 | |
| Number of cases of change from baseline | 287 | | 295 | |
| Mean value (standard deviation) | 0.7 | (22.83) | −3.0 | (20.50) |

| | | |
|---|---|---|
| Median | 0.0 | 0.0 |
| Min-Max | 96-84 | 82-73 |
| ANOVA two-sided P-value | 0.0446 | |
| Kruskal Wallis rank sum test for two-sided p-value | 0.0226 | |

MMRM model [1]

| | | | | |
|---|---|---|---|---|
| Least squares mean (standard error) | 1.3 | (1.17) | −3.5 | (1.16) |

| 95% two-sided confidence interval | (−1.0, 3.6) | | (−5.8, −1.3) |
|---|---|---|---|
| Least squares mean (standard error) of the difference over | 4.8 | (1.65) | |

| | |
|---|---|
| the vehicle group | |
| 95% two-sided confidence interval | (1.6, 8.1) |
| Bilateral P-value | 0.0035 |

Visit 4 VAS score

| Number of cases | 287 | | 294 | |
|---|---|---|---|---|
| Mean value (standard deviation) | 24.5 | (24.20) | 18.6 | (22.96) |
| Median | 14.0 | | 8.0 | |
| Min-Max | 0-100 | | 0-100 | |
| Number of cases of change from baseline | 287 | | 294 | |
| Mean value (standard deviation) | −3.8 | (23.67) | −6.9 | (20.71) |

| | | |
|---|---|---|
| Median | −2.0 | −2.0 |
| Min-Max | −97-90 | 76-61 |
| ANOVA two-sided P-value | 0.0923 | |
| Kruskal Wallis rank sum test for two-sided p-value | 0.1065 | |

MMRM model [1]

| | | | | |
|---|---|---|---|---|
| Least squares mean (standard error) | −3.2 | (1.12) | −7.7 | (1.10) |

| 95% two-sided confidence interval | (−5.4, −1.0) | | (−9.8, −5.5) |
|---|---|---|---|
| Least squares mean (standard error) of the difference over | 4.5 | (1.57) | |

| | |
|---|---|
| the vehicle group | |
| 95% two-sided confidence interval | (1.4, 7.5) |
| Bilateral P-value | 0.0046 |

Visit 5 VAS score

| Number of cases | 287 | | 295 | |
|---|---|---|---|---|
| Mean value (standard deviation) | 22.1 | (23.54) | 14.0 | (19.90) |
| Median | 13.0 | | 5.0 | |
| Min-Max | 0-98 | | 0-100 | |
| Number of cases of change from baseline | 287 | | 295 | |
| Mean value (standard deviation) | −6.3 | (25.54) | −11.8 | (22.39) |

| | | |
|---|---|---|
| Median | −3.0 | −5.0 |
| Min-Max | −98-89 | −88-53 |
| ANOVA two-sided P-value | 0.0061 | |
| Kruskal Wallis rank sum test for two-sided p-value | 0.0099 | |

MMRM model [1]

| | | | | |
|---|---|---|---|---|
| Least squares mean (standard error) | −5.6 | (1.12) | −12.3 | (1.11) |

| 95% two-sided confidence interval | (−7.9, −3.4) | | (−14.5, −10.2) |
|---|---|---|---|
| Least squares mean (standard error) of the difference over | 6.7 | (1.58) | |

| | |
|---|---|
| the vehicle group | |
| 95% two-sided confidence interval | (3.6, 9.8) |
| Bilateral P-value | <0.0001 |

MMRM = repeated measures mixed effects model.
Baseline was defined as the last non-missing data before the start of treatment with the trial drug.
[1] The actual observed change in VAS scores relative to baseline at each post-baseline visit was used as the dependent variable, treatment group, visit, and treatment group-visit interaction were the independent variables, and baseline VAS scores were used as covariates with an unstructured within-subject variance-covariance structure (UN).

TABLE 28
Summary of mean change from baseline in study eye fluorescein
staining score (ICSS) (full analysis set)

| | Cyclosporine A | |
|---|---|---|
| | Ophthalmic Gel | Vehicle |
| | (N = 315) | (N = 312) |
| Baseline ICSS score | | |
| Number of cases | 315 | 312 |
| Mean value (standard deviation) | 3.0 (0.79) | 3.0 (0.76) |
| Median | 3.0 | 3.0 |
| Min-Max | 2-4 | 2-4 |
| Visit 3 ICSS score | | |
| Number of cases | 314 | 310 |
| Mean value (standard deviation) | 2.2 (1.12) | 2.5 (1.10) |
| Median | 2.0 | 3.0 |
| Min-Max | 0-4 | 0-4 |
| Number of cases of change from baseline | 313 | 310 |
| Mean value (standard deviation) | −0.7 (0.99) | −0.5 (0.95) |
| Median | −1.0 | 0.0 |
| Min-Max | 4-2 | 4-2 |
| ANOVA two-sided P-value | 0.0010 | |
| Kruskal Wallis rank sum test for two- | 0.0018 | |
| sided p-value | | |
| MMRM model [1] | | |
| Least squares mean (standard error) | −0.7 (0.05) | −0.5 (0.05) |
| 95% two-sided confidence interval | (−0.8, −0.6) | (−0.6, −0.4) |
| Least squares mean (standard error) of | −0.3 (0.08) | |
| the difference over the vehicle group | | |
| 95% two-sided confidence interval | (−0.4, −0.1) | |
| Bilateral P-value | 0.0009 | |
| Visit 4 ICSS score | | |
| Number of cases | 305 | 303 |
| Mean value (standard deviation) | 2.0 (1.17) | 2.3 (1.22) |
| Median | 2.0 | 2.0 |
| Min-Max | 0-4 | 0-4 |
| Number of cases of change from baseline | 305 | 303 |
| Mean value (standard deviation) | −1.0 (1.06) | −0.7 (1.06) |
| Median | −1.0 | −1.0 |
| Min-Max | 4-2 | 4-2 |
| ANOVA two-sided P-value | <0.0001 | |
| Kruskal Wallis rank sum test for two- | <0.0001 | |
| sided p-value | | |
| MMRM model [1] | | |
| Least squares mean (standard error) | −1.0 (0.06) | −0.7 (0.06) |
| 95% two-sided confidence interval | (−1.2, −0.9) | (−0.8, −0.6) |
| Least squares mean (standard error) of | −0.4 (0.08) | |
| the difference over the vehicle group | | |
| 95% two-sided confidence interval | (−0.5, −0.2) | |
| Bilateral P-value | <0.0001 | |
| Visit 5 ICSS score | | |
| Number of cases | 298 | 301 |
| Mean value (standard deviation) | 1.7 (1.23) | 2.2 (1.31) |
| Median | 2.0 | 2.0 |
| Min-Max | 0-4 | 0-4 |
| Number of cases of change from baseline | 298 | 301 |
| Mean value (standard deviation) | −1.3 (1.13) | −0.8 (1.21) |
| Median | −1.0 | −1.0 |
| Min-Max | 4-2 | 4-2 |
| ANOVA two-sided P-value | <0.0001 | |
| Kruskal Wallis rank sum test for two- | <0.0001 | |
| sided p-value | | |
| MMRM model [1] | | |
| Least squares mean (standard error) | −1.3 (0.07) | −0.8 (0.07) |
| 95% two-sided confidence interval | (−1.4, −1.2) | (−0.9, −0.6) |
| Least squares mean (standard error) of | −0.5 (0.09) | |
| the difference over the vehicle group | | |
| 95% two-sided confidence interval | (−0.7, −0.3) | |
| Bilateral P-value | <0.0001 | |

Baseline was defined as the last non-missing data before the start of treatment with the trial drug.

TABLE 29
Summary of mean change from baseline in study eye fluorescein
staining score (ICSS) (consistent with protocol set)

| | Cyclosporine A | |
|---|---|---|
| | Ophthalmic Gel | Vehicle |
| | (N = 287) | (N = 295) |
| Baseline ICSS score | | |
| Number of cases | 287 | 295 |
| Mean value (standard deviation) | 3.0 (0.78) | 3.0 (0.76) |
| Median | 3.0 | 3.0 |
| Min-Max | 2-4 | 2-4 |
| Visit 3 ICSS score | | |
| Number of cases | 287 | 295 |
| Mean value (standard deviation) | 2.3 (1.12) | 2.5 (1.09) |
| Median | 2.0 | 3.0 |
| Min-Max | 0-4 | 0-4 |
| Number of cases of change from baseline | 287 | 295 |
| Mean value (standard deviation) | −0.7 (1.00) | −0.5 (0.95) |
| Median | −1.0 | 0.0 |
| Min-Max | 4-2 | 4-2 |
| ANOVA two-sided P-value | 0.0021 | |
| Kruskal Wallis rank sum test for two- | 0.0043 | |
| sided p-value | | |
| MMRM model [1] | | |
| Least squares mean (standard error) | −0.7 (0.06) | −0.5 (0.06) |
| 95% two-sided confidence interval | (−0.9, −0.6) | (−0.6, −0.4) |
| Least squares mean (standard error) of | −0.2 (0.08) | |
| the difference over the vehicle group | | |
| 95% two-sided confidence interval | (−0.4, −0.1) | |
| Bilateral P-value | 0.0024 | |
| Visit 4 ICSS score | | |
| Number of cases | 287 | 294 |
| Mean value (standard deviation) | 2.0 (1.16) | 2.3 (1.22) |
| Median | 2.0 | 2.0 |
| Min-Max | 0-4 | 0-4 |
| Number of cases of change from baseline | 287 | 294 |
| Mean value (standard deviation) | −1.0 (1.05) | −0.7 (1.05) |
| Median | −1.0 | −1.0 |
| Min-Max | 4-2 | 4-2 |
| ANOVA two-sided P-value | <0.0001 | |
| Kruskal Wallis rank sum test for two- | 0.0002 | |
| sided p-value | | |
| MMRM model [1] | | |
| Least squares mean (standard error) | −1.0 (0.06) | −0.7 (0.06) |
| 95% two-sided confidence interval | (−1.2, −0.9) | (−0.8, −0.6) |
| Least squares mean (standard error) of | −0.3 (0.09) | |
| the difference over the vehicle group | | |
| 95% two-sided confidence interval | (−0.5, −0.2) | |
| Bilateral P-value | <0.0001 | |
| Visit 5 ICSS score | | |
| Number of cases | 287 | 295 |
| Mean value (standard deviation) | 1.7 (1.23) | 2.2 (1.31) |
| Median | 2.0 | 2.0 |
| Min-Max | 0-4 | 0-4 |
| Number of cases of change from baseline | 287 | 295 |
| Mean value (standard deviation) | −1.3 (1.11) | −0.8 (1.22) |
| Median | −1.0 | −1.0 |
| Min-Max | 4-1 | 4-2 |
| ANOVA two-sided P-value | <0.0001 | |
| Kruskal Wallis rank sum test for two- | <0.0001 | |
| sided p-value | | |
| MMRM model [1] | | |
| Least squares mean (standard error) | −1.3 (0.07) | −0.8 (0.07) |
| 95% two-sided confidence interval | (−1.4, −1.2) | (−0.9, −0.7) |
| Least squares mean (standard error) of | −0.5 (0.09) | |
| the difference over the vehicle group | | |
| 95% two-sided confidence interval | (−0.7, −0.3) | |
| Bilateral P-value | <0.0001 | |

Baseline was defined as the last non-missing data before the start of treatment with the trial drug.

TABLE 30
Summary of Mean Change from Baseline in Study Eye Corneal
Staining Scores (Oxford Grading) (Full Analysis Set)

| | Cyclosporine A | |
|---|---|---|
| | Ophthalmic Gel | Vehicle |
| | (N = 315) | (N = 312) |
| Baseline Oxford grading score | | |
| Number of cases | 314 | 311 |
| Mean value (standard deviation) | 6.0 (3.68) | 6.3 (3.85) |
| Median | 5.0 | 5.0 |
| Min-Max | 0-15 | 0-15 |
| Visit 3 Oxford grading score | | |
| Number of cases | 312 | 307 |
| Mean value (standard deviation) | 4.9 (3.34) | 5.7 (3.68) |
| Median | 5.0 | 5.0 |
| Min-Max | 0-15 | 0-15 |
| Number of cases of change from baseline | 312 | 307 |
| Mean value (standard deviation) | −1.2 (3.35) | −0.7 (3.29) |
| Median | −1.0 | 0.0 |
| Min-Max | 10-9 | 10-11 |
| ANOVA two-sided P-value | 0.0375 | |
| Kruskal Wallis rank sum test for two- | 0.0186 | |
| sided p-value | | |
| Visit 4 Oxford grading score | | |
| Number of cases | 305 | 302 |
| Mean value (standard deviation) | 4.4 (3.27) | 5.3 (3.58) |
| Median | 4.0 | 5.0 |
| Min-Max | 0-15 | 0-15 |
| Number of cases of change from baseline | 305 | 302 |
| Mean value (standard deviation) | −1.7 (3.72) | −1.0 (3.47) |
| Median | −1.0 | −1.0 |
| Min-Max | 15-11 | 11-9 |
| ANOVA two-sided P-value | 0.0210 | |
| Kruskal Wallis rank sum test for two- | 0.0072 | |
| sided p-value | | |
| Visit 5 Oxford grading score | | |
| Number of cases | 298 | 301 |
| Mean value (standard deviation) | 4.0 (3.36) | 5.0 (3.63) |
| Median | 3.0 | 4.0 |
| Min-Max | 0-15 | 0-15 |
| Number of cases of change from baseline | 298 | 301 |
| Mean value (standard deviation) | −2.1 (3.57) | −1.3 (3.75) |
| Median | −2.0 | −1.0 |
| Min-Max | −12-10 | 13-9 |
| ANOVA two-sided P-value | 0.0076 | |
| Kruskal Wallis rank sum test for two- | 0.0029 | |
| sided p-value | | |

Baseline was defined as the last non-missing data before the start of treatment with the trial drug.

TABLE 31
Summary of mean change from baseline in the study eye's
corneoconjunctival staining score (Oxford grading)
(consistent with the protocol set)

| | Cyclosporine A | |
|---|---|---|
| | Ophthalmic Gel | Vehicle |
| | (N = 287) | (N = 295) |
| Baseline Oxford grading score | | |
| Number of cases | 287 | 295 |
| Mean value (standard deviation) | 6.0 (3.58) | 6.3 (3.85) |
| Median | 5.0 | 5.0 |
| Min-Max | 0-15 | 0-15 |
| Visit 3 Oxford grading score | | |
| Number of cases | 287 | 292 |
| Mean value (standard deviation) | 4.9 (3.39) | 5.7 (3.68) |
| Median | 5.0 | 5.0 |
| Min-Max | 0-15 | 0-15 |
| Number of cases of change from baseline | 287 | 292 |
| Mean value (standard deviation) | −1.2 (3.30) | −0.7 (3.29) |
| Median | −1.0 | 0.0 |
| Min-Max | 10-9 | 10-11 |
| ANOVA two-sided P-value | 0.0668 | |
| Kruskal Wallis rank sum test for two- | 0.0363 | |
| sided p-value | | |
| Visit 4 Oxford grading score | | |
| Number of cases | 287 | 293 |
| Mean value (standard deviation) | 4.4 (3.29) | 5.3 (3.55) |
| Median | 4.0 | 5.0 |
| Min-Max | 0-15 | 0-15 |
| Number of cases of change from baseline | 287 | 293 |
| Mean value (standard deviation) | −1.7 (3.72) | −1.0 (3.47) |
| Median | −1.0 | −1.0 |
| Min-Max | 15-11 | 11-9 |
| ANOVA two-sided P-value | 0.0206 | |
| Kruskal Wallis rank sum test for two- | 0.0062 | |
| sided p-value | | |
| Visit 5 Oxford grading score | | |
| Number of cases | 287 | 295 |
| Mean value (standard deviation) | 4.0 (3.39) | 5.0 (3.62) |
| Median | 3.0 | 4.0 |
| Min-Max | 0-15 | 0-15 |
| Number of cases of change from baseline | 287 | 295 |
| Mean value (standard deviation) | −2.1 (3.54) | −1.3 (3.74) |
| Median | −2.0 | −1.0 |
| Min-Max | −12-10 | 13-9 |
| ANOVA two-sided P-value | 0.0052 | |
| Kruskal Wallis rank sum test for two- | 0.0014 | |
| sided p-value | | |

Baseline was defined as the last non-missing data before the start of treatment with the trial drug.

TABLE 32
Summary of mean change from baseline in tear film break-up
time (BUT) measurements (full analysis set)

| | Cyclosporine A | |
|---|---|---|
| | Ophthalmic Gel | Vehicle |
| | (N = 315) | (N = 312) |
| Baseline BUT measurement value (s) | | |
| Number of cases | 315 | 312 |
| Mean value (standard deviation) | 3.319 (1.5730) | 3.402 (1.5412) |
| Median | 2.960 | 3.115 |
| Min-Max | 0.00-9.23 | 0.00-9.12 |
| Visit 3 BUT measurement value (s) | | |
| Number of cases | 313 | 310 |
| Mean value (standard deviation) | 3.817 (1.9309) | 3.816 (1.9122) |
| Median | 3.420 | 3.425 |
| Min-Max | 0.90-12.96 | 0.00-12.47 |
| >=10 | 6 (1.9%) | 3 (1.0%) |
| Number of cases of change from | 313 | 310 |
| baseline | | |
| Mean value (standard deviation) | 0.501 (1.8389) | 0.421 (1.6580) |
| Median | 0.370 | 0.200 |
| Min-Max | −6.87-9.94 | −4.18-8.22 |
| ANOVA two-sided P-value | 0.5710 | |
| Kruskal Wallis rank sum test for two- | 0.2882 | |
| sided p-value | | |
| Visit 4 BUT measurement value (s) | | |
| Number of cases | 303 | 303 |
| Mean value (standard deviation) | 3.962 (2.0453) | 3.904 (1.9546) |
| Median | 3.510 | 3.470 |
| Min-Max | 0.00-14.02 | 0.00-11.63 |
| >=10 | 8 (2.6%) | 2 (0.7%) |
| Number of cases of change from | 303 | 303 |
| baseline | | |
| Mean value (standard deviation) | 0.658 (2.0332) | 0.498 (1.8054) |
| Median | 0.480 | 0.380 |
| Min-Max | −6.61-9.43 | −5.57-7.38 |
| ANOVA two-sided P-value | 0.3071 | |
| Kruskal Wallis rank sum test for two- | 0.3643 | |
| sided p-value | | |
| Visit 5 BUT measurement value (s) | | |
| Number of cases | 298 | 301 |
| Mean value (standard deviation) | 4.416 (2.3535) | 4.507 (2.4282) |
| Median | 3.865 | 3.940 |
| Min-Max | 0.00-14.27 | 0.00-14.89 |
| >=10 | 8 (2.7%) | 10 (3.3%) |
| Number of cases of change from | 298 | 301 |
| baseline | | |
| Mean value (standard deviation) | 1.105 (2.3108) | 1.093 (2.2247) |
| Median | 0.920 | 0.800 |
| Min-Max | −5.35-10.80 | −5.54-10.28 |
| ANOVA two-sided P-value | 0.9512 | |
| Kruskal Wallis rank sum test for two- | 0.7086 | |
| sided p-value | | |

Baseline was defined as the last non-missing data before the start of treatment with the trial drug.
Percentages were calculated based on the number of cases of each visited subject in each treatment group in the full analysis set.

TABLE 33
Summary of mean change from baseline in tear film rupture
time (BUT) measurements (consistent with the protocol set)

| | Cyclosporine A | |
|---|---|---|
| | Ophthalmic Gel | Vehicle |
| | (N = 287) | (N = 295) |
| Baseline BUT measurement value (s) | | |
| Number of cases | 287 | 295 |
| Mean value (standard deviation) | 3.333 (1.5703) | 3.435 (1.5659) |
| Median | 2.980 | 3.170 |
| Min-Max | 0.00-9.23 | 0.00-9.12 |
| Visit 3 BUT measurement value (s) | | |
| Number of cases | 286 | 294 |
| Mean value (standard deviation) | 3.794 (1.9257) | 3.850 (1.9521) |
| Median | 3.410 | 3.440 |
| Min-Max | 0.90-12.96 | 0.00-12.47 |
| >=10 | 5 (1.7%) | 3 (1.0%) |
| Number of cases of change from | 286 | 294 |
| baseline | | |
| Mean value (standard deviation) | 0.462 (1.8554) | 0.425 (1.6838) |
| Median | 0.305 | 0.200 |
| Min-Max | −6.87-9.94 | −4.18-8.22 |
| ANOVA two-sided P-value | 0.8023 | |
| Kruskal Wallis rank sum test for two- | 0.5961 | |
| sided p-value | | |
| Visit 4 BUT measurement value (s) | | |
| Number of cases | 285 | 294 |
| Mean value (standard deviation) | 3.958 (2.0719) | 3.884 (1.9443) |
| Median | 3.470 | 3.445 |
| Min-Max | 0.00-14.02 | 0.00-11.63 |
| >=10 | 8 (2.8%) | 2 (0.7%) |
| Number of cases of change from | 285 | 294 |
| baseline | | |
| Mean value (standard deviation) | 0.617 (2.0406) | 0.451 (1.7785) |
| Median | 0.470 | 0.325 |
| Min-Max | −6.61-9.43 | −5.57-7.38 |
| ANOVA two-sided P-value | 0.2959 | |
| Kruskal Wallis rank sum test for two- | 0.3565 | |
| sided p-value | | |
| Visit 5 BUT measurement value (s) | | |
| Number of cases | 287 | 295 |
| Mean value (standard deviation) | 4.439 (2.3689) | 4.537 (2.4396) |
| Median | 3.890 | 3.960 |
| Min-Max | 0.00-14.27 | 0.00-14.89 |
| >=10 | 8 (2.8%) | 10 (3.4%) |
| Number of cases of change from | 287 | 295 |
| baseline | | |
| Mean value (standard deviation) | 1.106 (2.3268) | 1.102 (2.2430) |
| Median | 0.950 | 0.780 |
| Min-Max | −5.35-10.80 | −5.54-10.28 |
| ANOVA two-sided P-value | 0.9829 | |
| Kruskal Wallis rank sum test for two- | 0.7209 | |
| sided p-value | | |

Baseline was defined as the last non-missing data before the start of treatment with the trial drug.
Percentages were calculated based on the number of cases of each visited subject in each treatment group in the full analysis set.

TABLE 34
Summary of mean change from baseline in tear secretion
(Schirmer) test measurements (full analysis set)

| | Cyclosporine A | |
|---|---|---|
| | Ophthalmic Gel | Vehicle |
| | (N = 315) | (N = 312) |
| Baseline Schirmer test | | |
| measurement (mm/5 min) | | |
| Number of cases | 315 | 312 |
| Mean value (standard deviation) | 3.7 (2.44) | 3.8 (2.53) |
| Median | 3.0 | 3.0 |
| Min-Max | 0-9 | 0-9 |
| Visit 3 Schirmer test | | |
| measurement (mm/5 min) | | |
| Number of cases | 313 | 310 |
| Mean value (standard deviation) | 6.3 (5.68) | 5.5 (5.08) |
| Median | 5.0 | 4.0 |
| Min-Max | 0-34 | 0-30 |
| >=10 | 60 (19.2%) | 47 (15.2%) |
| Number of cases of change from | 313 | 310 |
| baseline | | |
| Mean value (standard deviation) | 2.6 (5.39) | 1.7 (4.79) |
| Median | 1.0 | 1.0 |
| Min-Max | 8-30 | 7-28 |
| ANOVA two-sided P-value | 0.0242 | |
| Kruskal Wallis rank sum test for two- | 0.0377 | |
| sided p-value | | |
| Visit 4 Schirmer test | | |
| measurement (mm/5 min) | | |
| Number of cases | 305 | 303 |
| Mean value (standard deviation) | 6.8 (5.93) | 6.5 (6.17) |
| Median | 5.0 | 5.0 |
| Min-Max | 0-35 | 0-30 |
| >=10 | 66 (21.6%) | 68 (22.4%) |
| Number of cases of change from | 305 | 303 |
| baseline | | |
| Mean value (standard deviation) | 3.1 (5.57) | 2.7 (6.05) |
| Median | 2.0 | 2.0 |
| Min-Max | 9-29 | 7-28 |
| ANOVA two-sided P-value | 0.4019 | |
| Kruskal Wallis rank sum test for two- | 0.2055 | |
| sided p-value | | |
| Visit 5 Schirmer test | | |
| measurement (mm/5 min) | | |
| Number of cases | 298 | 301 |
| Mean value (standard deviation) | 7.8 (6.97) | 6.5 (5.46) |
| Median | 6.0 | 5.0 |
| Min-Max | 0-35 | 0-30 |
| >=10 | 89 (29.9%) | 62 (20.6%) |
| Number of cases of change from | 298 | 301 |
| baseline | | |
| Mean value (standard deviation) | 4.1 (6.71) | 2.7 (5.34) |
| Median | 2.0 | 2.0 |
| Min-Max | 9-34 | 7-26 |
| ANOVA two-sided P-value | 0.0050 | |
| Kruskal Wallis rank sum test for two- | 0.0356 | |
| sided p-value | | |

Baseline was defined as the last non-missing data before the start of treatment with the trial drug.
Percentages were calculated based on the number of cases of each visited subject in each treatment group in the full analysis set.

TABLE 35
Summary of mean change from baseline in tear secretion
(Schirmer) test measurements (consistent with the protocol set)

| | Cyclosporine A | |
|---|---|---|
| | Ophthalmic Gel | Vehicle |
| | (N = 287) | (N = 295) |
| Baseline Schirmer test | | |
| measurement (mm/5 min) | | |
| Number of cases | 287 | 295 |
| Mean value (standard deviation) | 3.7 (2.44) | 3.8 (2.56) |
| Median | 3.0 | 3.0 |
| Min-Max | 0-9 | 0-9 |
| Visit 3 Schirmer test | | |
| measurement (mm/5 min) | | |
| Number of cases | 287 | 295 |
| Mean value (standard deviation) | 6.2 (5.60) | 5.5 (5.14) |
| Median | 5.0 | 4.0 |
| Min-Max | 0-34 | 0-30 |
| >=10 | 54 (18.8%) | 46 (15.6%) |
| Number of cases of change from | 287 | 295 |
| baseline | | |
| Mean value (standard deviation) | 2.5 (5.35) | 1.6 (4.81) |
| Median | 1.0 | 1.0 |
| Min-Max | 8-30 | 7-28 |
| ANOVA two-sided P-value | 0.0400 | |
| Kruskal Wallis rank sum test for two- | 0.0517 | |
| sided p-value | | |
| Visit 4 Schirmer test | | |
| measurement (mm/5 min) | | |
| Number of cases | 287 | 294 |
| Mean value (standard deviation) | 6.8 (6.03) | 6.5 (6.24) |
| Median | 5.0 | 5.0 |
| Min-Max | 0-35 | 0-30 |
| >=10 | 63 (22.0%) | 68 (23.1%) |
| Number of cases of change from | 287 | 294 |
| baseline | | |
| Mean value (standard deviation) | 3.1 (5.68) | 2.7 (6.11) |
| Median | 2.0 | 1.0 |
| Min-Max | 9-29 | 7-28 |
| ANOVA two-sided P-value | 0.3793 | |
| Kruskal Wallis rank sum test for two- | 0.1839 | |
| sided p-value | | |
| Visit 5 Schirmer test | | |
| measurement (mm/5 min) | | |
| Number of cases | 287 | 295 |
| Mean value (standard deviation) | 7.7 (7.01) | 6.4 (5.49) |
| Median | 6.0 | 5.0 |
| Min-Max | 0-35 | 0-30 |
| >=10 | 83 (28.9%) | 61 (20.7%) |
| Number of cases of change from | 287 | 295 |
| baseline | | |
| Mean value (standard deviation) | 4.0 (6.76) | 2.6 (5.34) |
| Median | 2.0 | 2.0 |
| Min-Max | 9-34 | 7-26 |
| ANOVA two-sided P-value | 0.0060 | |
| Kruskal Wallis rank sum test for two- | 0.0429 | |
| sided p-value | | |

Baseline was defined as the last non-missing data before the start of treatment with the trial drug.
Percentages were calculated based on the number of cases of each visited subject in each treatment group in the full analysis set.

### 16.3 Adverse Events

A total of 224 TEAEs were reported by 127 (39.6%) subjects in the cyclosporine A ophthalmic gel group, of which 44 (13.7%) subjects reported a total of 71 TEAEs related to the experimental drug; 96 (30.6%) subjects in the vehicle group reported a total of 162 TEAEs, of which 20 (6.4%) subjects reported a total of 37 TEAEs related to the experimental drug. The overall incidence of TEAE and the incidence of TEAE associated with the trial drug were higher in the cyclosporine A ophthalmic gel group than in the vehicle group, and the incidence of TEAE associated with the trial drug was higher in the vehicle group.

In the SS, 127 (39.6%) participants in the CyclAGel group reported 224 TEAEs, including 44 (13.7%) participants reporting 71 TEAEs related to the study drug. In addition, 96 (30.6%) participants in the vehicle group reported 162 TEAEs, including 20 (6.4%) participants reporting 37 TEAEs related to the study drug. The most common TEAEs in all participants were eye pain, eye foreign body sensation, vision loss, and urinary tract infection (Table 36). Drug discontinuation due to TEAEs in the two groups was observed in five (1.6%) and three (1.0%) participants, respectively. The incidences of SAE were six (1.9%) and two (0.6%), respectively. All SAEs were non-ocular and irrelevant to the study drugs. During the study period, the average compliance was 98.9% and 99.0% in the CyclAGel and vehicle groups, respectively.

The majority of TEAEs were moderate. Only 4 (1.2%) subjects in the cyclosporine A ophthalmic gel group reported 5 cases of severe TEAE, of which 2 (0.6%) reported 3 cases of severe TEAE associated with the test drug; only 2 (0.6%) subjects in the vehicle group reported 3 cases of severe TEAE, of which 1 (0.3%) reported 1 case of severe TEAE associated with the test drug. The incidence of severe TEAE and the incidence of severe TEAE associated with the experimental drug were similar in the cyclosporine A ophthalmic gel group and the vehicle group.

The incidence of TEAE leading to trial drug suspension was similar in the cyclosporine A ophthalmic gel group to that in the vehicle group, with a total of 8 (2.5%) subjects reporting 11 cases of TEAE leading to trial drug suspension and 6 (1.9%) subjects reporting 8 cases of TEAE leading to trial drug suspension. The rate of TEAE leading to trial drug suspension was similar in the cyclosporine A ophthalmic gel group and the vehicle group.

The incidence of TEAEs leading to trial drug discontinuation was similar in the cyclosporine A ophthalmic gel group and the vehicle group with 3 (1.0%) subjects reporting 4 TEAEs leading to trial drug discontinuation.

Six TEAEs leading to subject withdrawal were reported by 5 (1.6%) subjects in the cyclosporine A ophthalmic gel group, of which 3 (0.9%) subjects reported 4 TEAEs related to the experimental drug leading to subject withdrawal, and 4 TEAEs leading to subject withdrawal were reported by 3 (1.0%) subjects in the vehicle group, of which 1 (0.3%) subject reported 1 TEAE related to the experimental drug leading to subject withdrawal. The incidence of TEAEs leading to subject withdrawal from the study and the incidence of TEAEs leading to subject withdrawal associated with the experimental drug and the experimental drug was similar to vehicle in the cyclosporine A ophthalmic gel group.

A total of 12 SAEs were reported in 8 subjects: 9 in 6 (1.9%) subjects in the cyclosporine A ophthalmic gel group and 3 in 2 (0.6%) subjects in the vehicle group. There were no SAEs related to the test drug and no TEAEs resulting in death in the entire trial.

The results showed that the overall incidence of TEAE and the incidence of trial drug-related TEAE and trial drug-related TEAE were higher in the cyclosporine A ophthalmic gel group than in the vehicle group, but the incidence of other severe types of TEAE, TEAE leading to suspension or termination of trial drug use, TEAE leading to suspension or termination of trial drug use, TEAE leading to withdrawal of subjects from the study, and SAE were not significantly different in the cyclosporine A ophthalmic gel group than in the vehicle group.

TABLE 36
Incidence of Treatment-Emergent Adverse Events (TEAEs)

| | CyclAGel 0.05% | Vehicle |
|---|---|---|
| TEAEs | (n = 321) | (n = 314) |
| Overall TEAEs | 127 (39.6%) | 96 (30.6%) |
| Ocular | 63 (19.6%) | 46 (14.6%) |
| Non-ocular | 81 (25.2%) | 59 (18.8%) |
| Severe TEAEs | 4 (1.2%) | 2 (0.6%) |
| Ocular | 2 (0.6%) | 1 (0.3%) |
| Non-ocular | 2 (0.6%) | 1 (0.3%) |
| TEAEs related to the study drug | 44 (13.7%) | 20 (6.4%) |
| Ocular | 40 (12.5%) | 17 (5.4%) |
| Non-ocular | 5 (1.6%) | 3 (1.0%) |
| TEAEs leading to drug | 5 (1.6%) | 3 (1.0%) |
| discontinuation | | |
| TEAEs leading to death | 0 | 0 |
| SAEs | 6 (1.9%) | 2 (0.6%) |
| SAEs related to the study drug | 0 | 0 |
| TEAEs ≥ 1% in either group | | |
| Eye pain | 26 (8.1%) | 7 (2.2%) |
| Eye foreign body sensation | 7 (2.2%) | 4 (1.3%) |
| Eye irritation | 7 (2.2%) | 1 (0.3%) |
| Vision loss | 4 (1.2%) | 6 (1,9%) |
| Urinary tract infection | 4 (1.2%) | 5 (1.6%) |
| Eye discomfort | 4 (1.2%) | 3 (1.0%) |
| Dry eye | 4 (1.2%) | 2 (0.6%) |
| Increased tears | 4 (1.2%) | 2 (0.6%) |
| Nasopharyngitis | 2 (0.6%) | 6 (1.9%) |
| Blurred vision | 2 (0.6%) | 3 (1.0%) |
| Upper respiratory infection | 2 (0.6%) | 3 (1.0%) |
| Chest discomfort | 1 (0.3%) | 3 (1.0%) |
| Pharyngitis | 0 | 3 (1.0%) |

Abbreviation: SAE, serious adverse event.

TABLE 38
Summary of overall TEAEs related to experimental drugs
by MedDRA system organ classification, preferred
terminology (safety data set)

| | Cyclosporine A | |
|---|---|---|
| | Ophthalmic Gel | Vehicle |
| System organ classification | (N = 321) | (N = 314) |
| Preferred Terminology | n (%) | n (%) |
| Overall TEAE related to the test drug | 44 (13.7%) | 20 (6.4%) |
| Eye Organ Diseases | 40 (12.5%) | 17 (5.4%) |
| Eye pain | 22 (6.9%) | 5 (1.6%) |
| Eye Stimulation | 7 (2.2%) | 1 (0.3%) |
| Eye foreign body sensation | 6 (1.9%) | 2 (0.6%) |
| Dry eye | 4 (1.2%) | 1 (0.3%) |
| Eye discomfort | 4 (1.2%) | 3 (1.0%) |
| Increased tearing | 3 (0.9%) | 2 (0.6%) |
| Blurred vision | 2 (0.6%) | 3 (1.0%) |
| Pruritus of the eyes | 2 (0.6%) | 0 |
| Conjunctival hemorrhage | 1 (0.3%) | 0 |
| Reduced vision | 1 (0.3%) | 1 (0.3%) |
| photophobia | 1 (0.3%) | 0 |
| Eye congestion | 1 (0.3%) | 0 |
| Eye discharge | 1 (0.3%) | 1 (0.3%) |
| eye swelling | 1 (0.3%) | 0 |
| Blepharitis | 1 (0.3%) | 0 |
| Ulcerative keratitis | 0 | 1 (0.3%) |
| Visual fatigue | 0 | 1 (0.3%) |
| Kidney and urinary tract diseases | 2 (0.6%) | 0 |
| Hematuria | 2 (0.6%) | 0 |
| Liver and biliary system diseases | 1 (0.3%) | 0 |
| Abnormal liver function | 1 (0.3%) | 0 |
| Infectious and Infectious Diseases | 1 (0.3%) | 2 (0.6%) |
| Chronic sinusitis | 1 (0.3%) | 0 |
| Upper respiratory tract infection | 0 | 1 (0.3%) |
| Periodontitis | 0 | 1 (0.3%) |
| Respiratory, thoracic and mediastinal | 1 (0.3%) | 1 (0.3%) |
| diseases | | |
| runny nose | 1 (0.3%) | 0 |
| Allergic rhinitis | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

TABLE 39
Summary of ocular TEAEs related to experimental drugs
by MedDRA system organ classification, preferred
terminology (safety data set)

| | Cyclosporine A | |
|---|---|---|
| | Ophthalmic Gel | Vehicle |
| System organ classification | (N = 321) | (N = 314) |
| Preferred Terminology | n (%) | n (%) |
| Ocular TEAE related to the test drug | 40 (12.5%) | 17 (5.4%) |
| Eye Organ Diseases | 40 (12.5%) | 17 (5.4%) |
| Eye pain | 22 (6.9%) | 5 (1.6%) |
| Eye Stimulation | 7 (2.2%) | 1 (0.3%) |
| Eye foreign body sensation | 6 (1.9%) | 2 (0.6%) |
| Dry eye | 4 (1.2%) | 1 (0.3%) |
| Eye discomfort | 4 (1.2%) | 3 (1.0%) |
| Increased tearing | 3 (0.9%) | 2 (0.6%) |
| Blurred vision | 2 (0.6%) | 3 (1.0%) |
| Pruritus of the eyes | 2 (0.6%) | 0 |
| Conjunctival hemorrhage | 1 (0.3%) | 0 |
| Reduced vision | 1 (0.3%) | 1 (0.3%) |
| photophobia | 1 (0.3%) | 0 |
| Eye congestion | 1 (0.3%) | 0 |
| Eye discharge | 1 (0.3%) | 1 (0.3%) |
| eye swelling | 1 (0.3%) | 0 |
| Blepharitis | 1 (0.3%) | 0 |
| Ulcerative keratitis | 0 | 1 (0.3%) |
| Visual fatigue | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

TABLE 40
Summary of non-ocular TEAEs related to experimental drugs
by MedDRA system organ classification, preferred
terminology (safety data set)

| | Cyclosporine A | |
|---|---|---|
| | Ophthalmic Gel | Vehicle |
| System organ classification | (N = 321) | (N = 314) |
| Preferred Terminology | n (%) | n (%) |
| Non-eye TEAE related to the test drug | 5 (1.6%) | 3 (1.0%) |
| Kidney and urinary tract diseases | 2 (0.6%) | 0 |
| Hematuria | 2 (0.6%) | 0 |
| Liver and biliary system diseases | 1 (0.3%) | 0 |
| Abnormal liver function | 1 (0.3%) | 0 |
| Infectious and Infectious Diseases | 1 (0.3%) | 2 (0.6%) |
| Chronic sinusitis | 1 (0.3%) | 0 |
| Upper respiratory tract infection | 0 | 1 (0,3%) |
| Periodontitis | 0 | 1 (0.3%) |
| Respiratory, thoracic and mediastinal | 1 (0.3%) | 1 (0.3%) |
| diseases | | |
| runny nose | 1 (0.3%) | 0 |
| Allergic rhinitis | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

TABLE 41
Summary of heavy overall TEAE related to experimental
drugs by MedDRA system
organ classification, preferred terminology (safety data set)

| | Cyclosporine A | |
|---|---|---|
| | Ophthalmic Gel | Vehicle |
| System organ classification | (N = 321) | (N = 314) |
| Preferred Terminology | n (%) | n (%) |
| Heavy overall TEAE related to | 2 (0.6%) | 1 (0.3%) |
| the test drug | | |
| Eye Organ Diseases | 2 (0.6%) | 1 (0.3%) |
| Eye pain | 2 (0.6%) | 0 |
| Ulcerative keratitis | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

TABLE 42
Summary of severe ocular TEAE related to
experimental drugs by MedDRA system organ
classification, preferred terminology (safety data set)

| | Cyclosporine A | |
|---|---|---|
| | Ophthalmic Gel | Vehicle |
| System organ classification | (N = 321) | (N = 314) |
| Preferred Terminology | n (%) | n (%) |
| Severe ocular TEAE related | 2 (0.6%) | 1 (0.3%) |
| to the test drug | | |
| Eye Organ Diseases | 2 (0.6%) | 1 (0.3%) |
| Eye pain | 2 (0.6%) | 0 |
| Ulcerative keratitis | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

TABLE 43
Summary of severe non-ocular TEAE related
to experimental drugs by MedDRA system
organ classification, preferred terminology (safety data set)

| | Cyclosporine A | |
|---|---|---|
| | Ophthalmic Gel | Vehicle |
| System organ classification | (N = 321) | (N = 314) |
| Preferred Terminology | n (%) | n (%) |
| Severe non-ocular TEAE | 0 | 0 |
| related to the test drug | | |

TEAE = Treatment/medication-emergent or worsening adverse event.
Adverse events were coded using MedDRA 24.0.
Percentages were calculated based on the number of subjects in each treatment group in the safety data set.

TABLE 44
Summary of overall SAEs during treatment according to MedDRA system organ
classification, preferred terminology (safety data set)

| | Cyclosporine A | |
|---|---|---|
| | Ophthalmic Gel | |
| System organ classification | (N = 321) | Vehicle (N = 314) |
| Preferred Terminology | n (%) | n (%) |
| Overall SAE during treatment | 6 (1.9%) | 2 (0.6%) |
| Infectious and Infectious Diseases | 1 (0.3%) | 0 |
| Gastroenteritis | 1 (0.3%) | 0 |
| All kinds of neurological diseases | 1 (0.3%) | 1 (0.3%) |
| Embolic cerebral infarction | 1 (0.3%) | 0 |
| Cerebral infarction | 0 | 1 (0.3%) |
| Various types of injuries, poisoning and operational | 1 (0.3%) | 0 |
| complications | | |
| Fracture of the fibula | 1 (0.3%) | 0 |
| Various musculoskeletal and connective tissue diseases | 1 (0.3%) | 0 |
| Osteoarthritis | 1 (0.3%) | 0 |
| Kidney and urinary tract diseases | 1 (0.3%) | 0 |
| Kidney stone disease | 1 (0.3%) | 0 |
| Hydronephrosis | 1 (0.3%) | 0 |
| Ureteral calculus | 1 (0.3%) | 0 |
| Gastrointestinal system diseases | 1 (0.3%) | 0 |
| Hemorrhoids | 1 (0.3%) | 0 |
| Vascular and Lymphatic Vessel Diseases | 1 (0.3%) | 0 |
| High blood pressure | 1 (0.3%) | 0 |
| Psychiatric category | 0 | 1 (0.3%) |
| Sleep disorders | 0 | 1 (0.3%) |
| Heart Organ Disease | 0 | 1 (0.3%) |
| Unstable angina pectoris | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

TABLE 45
Summary of Ocular SAEs During Treatment by MedDRA System
Organ Classification, Preferred Terminology (Safety Data Set)

| | Cyclosporine A | |
|---|---|---|
| | Ophthalmic Gel | Vehicle |
| System organ classification | (N = 321) | (N = 314) |
| Preferred Terminology | n (%) | n (%) |
| Ocular SAE during treatment | 0 | 0 |

TEAE = Treatment/medication-emergent or worsening adverse event.
Adverse events were coded using MedDRA 24.0.
Percentages were calculated based on the number of subjects in each treatment group in the safety data set.

TABLE 46
Summary of Non-Ocular SAEs During Treatment by MedDRA System Organ
Classification, Preferred Terminology (Safety Data Set)

| | Cyclosporine A | |
|---|---|---|
| | Ophthalmic Gel | |
| System organ classification | (N = 321) | Vehicle (N = 314) |
| Preferred Terminology | n (%) | n (%) |
| Non-ocular SAE during treatment | 6 (1.9%) | 2 (0.6%) |
| Infectious and Infectious Diseases | 1 (0.3%) | 0 |
| Gastroenteritis | 1 (0.3%) | 0 |
| All kinds of neurological diseases | 1 (0.3%) | 1 (0.3%) |
| Embolic cerebral infarction | 1 (0.3%) | 0 |
| Cerebral infarction | 0 | 1 (0.3%) |
| Various types of injuries, poisoning and operational | 1 (0.3%) | 0 |
| complications | | |
| Fracture of the fibula | 1 (0.3%) | 0 |
| Various musculoskeletal and connective tissue diseases | 1 (0.3%) | 0 |
| Osteoarthritis | 1 (0.3%) | 0 |
| Kidney and urinary tract diseases | 1 (0.3%) | 0 |
| Kidney stone disease | 1 (0.3%) | 0 |
| Hydronephrosis | 1 (0.3%) | 0 |
| Ureteral calculus | 1 (0.3%) | 0 |
| Gastrointestinal system diseases | 1 (0.3%) | 0 |
| Hemorrhoids | 1 (0.3%) | 0 |
| Vascular and Lymphatic Vessel Diseases | 1 (0.3%) | 0 |
| High blood pressure | 1 (0.3%) | 0 |
| Psychiatric category | 0 | 1 (0.3%) |
| Sleep disorders | 0 | 1 (0.3%) |
| Heart Organ Disease | 0 | 1 (0.3%) |
| Unstable angina pectoris | 0 | 1 (0.3%) |

TEAE = Treatment/medication-emergent or worsening adverse event.
Adverse events were coded using MedDRA 24.0.
Percentages were calculated based on the number of subjects in each treatment group in the safety data set.

### 16.4 Discussion

The primary efficacy endpoint was met: the proportion of subjects in the cyclosporine A ophthalmic gel group with a decrease in corneal fluorescein staining score (ICSS) of: ≥1 point from baseline at visit 5 was significantly greater than in the vehicle group (73.7% vs. 53.2%). The treatment effect on ICSS in the cyclosporine A ophthalmic gel group also showed a trend that was generally consistent with the overall treatment effect in the PPS subjects when the analysis of the main efficacy indicators was repeated in the PPS subjects, and in the different subgroups. Treatment with cyclosporine A ophthalmic gel once daily for 84 days significantly reduced (below) corneal fluorescein staining scores in patients with moderate-to-severe dry eyes.

It is worth noting that the average compliance to the experimental drugs in this study was 98.94%. Compared to the marketed drugs, Cyclosporine A ophthalmic gel is only needed once daily at bedtime, reducing the number of intermittent doses for dry eye patients and greatly improving patient compliance.

In summary, the results of this study showed that cyclosporine A ophthalmic gel was significantly more effective than vehicle in the treatment of patients with moderate-to-severe dry eyes.

The COSMO phase III clinical trial aimed to confirm the efficacy and safety of CyclAGel in treating patients with moderate-to-severe DED. More subjects in the CyclAGel group (73.7%) than in the vehicle group (53.2%) had at least a 1-point improvement in ICSS from baseline to day 84 (end of the study), achieving the primary efficacy endpoint. Moreover, the changes from baseline to day 84 in ICSS, Oxford scale scoring of corneal and conjunctival fluorescein staining, and Schirmer tear test in the CyclAGel group were all significantly better than in the vehicle group. These results suggest that CyclAGel improves the DED signs and symptoms in patients with moderate-to-severe DED. In addition, it has good safety and patient tolerance.

Previous studies reported that the efficacy of CsA ophthalmic preparations was superior to vehicles in patients with DED, which was consistent with the main findings of this study.

One of the challenges for topical administration of cyclosporine to the eyes is the high hydrophobicity, which impedes the use of common aqueous ophthalmic vehicles. Therefore, cyclosporine is usually dissolved in olive oil or oil-based emulsions, which are poorly tolerated and result in low ocular availability because of the short retention time on the ocular surface. CyclAGel dissolves cyclosporine to form a transparent stable hydrogel so that it is directly available to the ocular surface, which is in contrast to emulsions requiring liberation from micelles. The CyclAGel in the present study is the first hydrogel preparation of CsA. Carbomer in the vehicle acts as a stabilizing agent, has good adherence to the ocular surface, and does not induce ocular irritation. In addition, CyclAGel in cornea, conjunctiva, and tears showed superior Cmax and area under the concentration-time curve than Restasis. Thus, the effective concentration of CyclAGel in conjunctiva and cornea could be maintained over a long time. High bioavailability and long effective concentration maintenance time enable CyclAGel to achieve good efficacy.

Notably, the baseline ICSS in the present study was 3.0, indicating moderate-to-severe corneal epithelial defect. On day 84, the mean ICSS improved to 1.7 (mild corneal epithelial defect) in the CyclAGel group. A severe corneal epithelial defect is difficult to recover from, while CyclAGel 0.05% accelerate severe corneal epithelial defects healing and can shift moderate-to-severe to mild corneal epithelial defects within 12 weeks. Lifitegrast®, a small molecule LFA-1 antagonist, was approved by the FDA for treating patients with DED in July 2016. The proportion of patients with at least a 1-point improvement in ICSS from baseline to day 84 was 22.2% in the Lifitegrast group (mean baseline ICSS of 1.84) in the OPUS-1 study, compared with 73.7% in the CyclAGel group and 69.7% in the subgroup with a baseline ICSS of 2 in the present study. In other words, CyclAGel 0.05% once every night showed promising efficacy for corneal epithelial defects healing in moderate-to-severe DED. Furthermore, real-world experience showed that improvements in DED signs and symptoms were evident at 3 months and up to 12 months after Lifitegrast initiation, and 0.1% CsA cationic emulsion usually take 3-6 months to show efficacy against DED signs and symptoms, while the onset time of CyclAGel 0.05% was within 2 weeks. Nevertheless, formal comparisons among different drugs for DED require head-to-head trials.

Regarding the relief of DED symptoms, both groups showed improvements compared with baseline. The vehicle also contained lubricants that might help alleviate the symptoms, which might be why the CyclAGel group showed no statistically significant difference over the vehicle group for some symptoms. Meanwhile, symptoms are quite variable and subjective indicators, especially in severe DED patients. Subjects in the present study had moderate-to-severe corneal epithelial defects, which might increase the variability of symptoms. Modification to collecting data on symptoms is needed in future studies to increase the accuracy. EDS, at least, improved significantly from baseline (approximately 40%). The phase II study of 0.05% CyclAGel once daily showed that the change from baseline in EDS scores after 84 days of treatment was −29.17±23.77, 20 consistent with a mean value of −31 (range, −94 to 23) in the present phase III trial.

According to the subgroup analyses, female <65 years were more likely to benefit from CyclAGel. The major risk factors for DED are age, sex, androgen deficiency, and menopause. As the proportion of menopausal females and males with decreased androgen levels will be higher in the participants >65 years group than in the participants <65 years age group, there is probably a higher likelihood of intractable DED in older participants, especially in menopausal females.

Regarding safety, the rates of overall TEAEs and TEAEs related to the study drug were lower than those of the phase II study. The most common TEAEs were eye pain (8.1%), eye foreign body sensation (2.2%), and eye irritation (2.2%) in the CyclAGel group. Most of the TEAEs were mild/moderate. Eight subjects reported SAEs, which were all ruled to be unrelated to the study drug. The overall safety characteristics were consistent with the phase II trial of CyclAGel and clinical trials of other CsA ophthalmic formulations. No new safety signals were observed. In addition, instillation site pain was reported in 24.2% of patients receiving OTX-101 0.09% solution and in 22% of patients receiving Lifitegrast. In contrast, no instillation site pain was reported in the CyclAGel group, indicating better patient comfort. The average patient compliance during the study period was above 98%, which might be related to less frequent medication use (only once every night) and better patient comfort. Still, the safety profile should be compared with other topical medications for DED in the future. In addition, DED is a chronic condition requiring long-term treatment. The stable, homogeneous, and clear nature of CyclAGel, along with its frequency of use of only once a day, makes it an easy-to-use ophthalmic formulation that ensures better long-term adherence, thus helping patients improve their quality of life.

In conclusion, CyclAGel 0.05% QD significantly reduced corneal and conjunctival staining and improved tear secretion compared with the control group in moderate-to-severe DED. It also significantly alleviated the symptoms. CyclAGel 0.05% QD is a new effective, safe, and well-tolerated therapeutic option that might bring additional benefits of convenience and compliance as a once-A-day treatment for moderate-to-severe DED.

### 16.5 Appendix

### 1. VAS Score for Dry Eye Symptoms

VAS score for dry eye symptoms is used to assess the severity of the patient's disease. The score includes seven categories of dryness, burning sensation/stinging sensation, itching, foreign body sensation, discomfort, photophobia and pain.

Visual analogue scale (VAS) is used for scoring. VAS is a 100 mm long straight line marked with very comfortable and very uncomfortable at both ends. (FIG. 1) Subjects will mark a point on the straight line according to their comfort degree, and the length from the starting point to the mark is the amount of comfort. This method is simple and easy to implement, relatively objective and sensitive. When in use, the graduated side faces away from the subject, and the investigator assigns a score according to the position marked by the subject.

At each visit, the subjects are asked in detail about their dry eye symptoms in the last two weeks and draw their discomfort for each of the seven items. Then the investigator assigns a score according to the position marked by the subject. 0 point represents no discomfort, 100 points represents the greatest discomfort, keep the integer, and the higher the value, the more severe the symptoms.

### 2. Slit Lamp Microscopy

Slit lamp is an indispensable tool for anterior segment examination. It can not only see superficial lesions clearly, but also adjust the width of the focus and light source to form an optical section and see the deep tissue and the front-to-back relationship.

It is equipped with a preset lens, contact lens, gonioscope and three-mirror contact lens, which can check the anterior chamber angle, vitreous body and fundus. It is equipped with anterior chamber depth gauge, applanation tonometer, camera, and its use is more extensive.

### Before Operation

Environment preparation: relatively dark room

Instrument preparation: basic reset, including adjust light knife length to 8 or 9, width to 0, eyepiece focal power to 0, release the lock wheel, and set magnification to low power.

Examiner's preparation: wear a white lab coat or overalls and wash hands.

Preparation of the subject: understand the medical history, make a general eye examination first.

### Operation Steps

Adjust the seat height and position according to the patient's body shape, so that the examiner and the examinee are in a comfortable position. The examinee removes the frame glasses, and the examiner instructs the examinee to place the forehead and mandible on the forehead rest and lower base respectively, and adjust the height so that the lateral canthal height is at the level of the eye line.

Explain to the patient appropriately before the examination, and ask to look at the indicator light or look directly into the microscope during the examination.

Instruct the examinee to close their eyes, turn on the lighting system, and adjust the components so that the slit lamp and the microscope are at an angle of 30° to 50°, and the light enters from the temporal side. Use the examinee's eyelashes or nose bridge as the focus target, adjust the focus, and then adjust the binocular microscope to make the distance consistent with the examiner's interpupillary distance.

According to the situation of the subject, different lighting methods are used for inspection. For standard operation, the eye condition of the examinee needs to be checked from front to back. First adjust the width of the light knife to the widest, observe the eyelashes, let the patient open the eyes, look straight ahead, and check the general condition of the cornea and conjunctiva; then adjust the width of the light knife to observe the eyelids, eyelid margin, meibomian glands, conjunctiva, and cornea.

### After Operation

Tidy and clean materials, turn off the power in time, and return the materials to their original places. The examiner needs to wash hands with disinfectant or hand sanitizer; if there is a problem with the inspection instrument, it should be adjusted and repaired in time to ensure the accuracy of the results of the slit lamp microscopy.

### 3. Determination of Break-Up Time (BUT)

Objective: To quantitatively evaluate the stability and quality of the tear film.

Determination method: Take out the test paper for corneal and conjunctival staining of the ocular surface, damp the stained part of the test paper with 1-2 drops of sterile intraocular irrigating solution or normal saline, and gently touch the wet part on the subject's cornea and conjunctiva. Instruct the subject to blink 3 to 5 times, and record the time from when the eyes were opened naturally after the last blink to the first dark spot on the cornea. Measure 3 times with an electronic stopwatch and take the mean value.

Meaning: BUT<10 s means that the break-up time is shortened, which can be diagnose as dry eye; BUT>10 s means normal.

### 4. ICSS Score

Scoring method: only evaluate the lower ⅓ of cornea, and use the 4-point method: no staining is 0, few/rare punctate lesions is 1 point, discrete and countable lesions (easy to count) is 2 points, lesions too numerous to count, but not coalescent is 3 points and coalescent is 4 points. (ICSS: no staining=0, few/rare punctate lesions=1, discrete and countable lesions=2, lesions too numerous to count, but not coalescent=3, coalescent=4) (FIG. 2)

### 5. Corneal and Conjunctival Staining and Photography

Examination method: Take out the test paper for corneal and conjunctival staining of the ocular surface, damp the stained part of the test paper with 1-2 drops of normal saline, and gently touch the wet part on the subject's conjunctiva.

Observe and take pictures of corneal fluorescein staining, observe and take pictures of conjunctival lissamine green staining.

### 6. Oxford Grading Scale

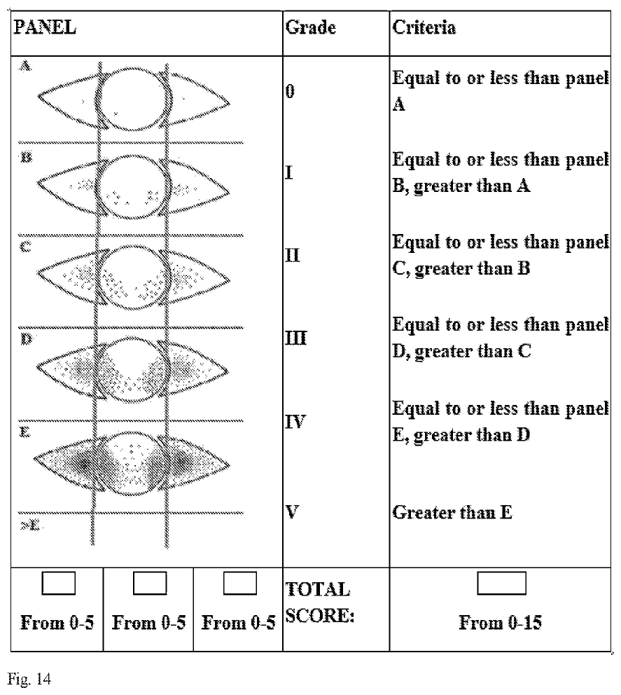

The scoring method for the Oxford grading scale is as follows: compare the stained cornea, nasal conjunctiva, and temporal conjunctiva, and assign score against the diagram. Each part has a score of 0˜5. Add the scores of the 3 parts to get the total score, which is divided into 6 levels: 0, I, II, III, IV, and V. A total score of 0 is divided into level 0, a total score of 1˜3 is divided into level I, a total score of 4˜6 is divided into level II, a total score of 7˜9 is divided into level III, and a total score of 10˜12 is divided into level IV. 13˜15 are divided into level V. (FIG. 14)

### 7. Basic Tear Secretion Test—Schirmer Test (Surface Anesthesia)

Objective: To measure basal tear secretion.

The examination method includes:
1. The subject is in a dark room, the lower vault of conjunctiva is dripped with anesthetic, the Schirmer test is performed 30 seconds later.
2. Measure basal tear secretion by excluding light and filter paper stimulus factor.
3. The examiner folds one end of the standard filter paper 5 mm into a right angle, and places the folded end in the conjunctiva on the lateral ⅓ of the patient's lower eyelid, and the other end hangs outside the eyelid;
4. Instruct the patient to lightly close eyes and look up slightly, and blink at will;
5. Gently pull the lower lid and take out the filter paper strip at 5 minutes, and record its length after standing for 2 minutes.

Meaning: ≥10 mm/5 min is negative; <10 mm/5 min is positive.

### 17 Abbreviation List

TABLE 47
Abbreviation List

| | |
|---|---|
| AE | Adverse event |
| ALT | Glutamic-pyruvic transaminase |
| AST | Glutamic oxalacetic transaminase |
| BUN | Urea nitrogen |
| BUT | Break-up time |
| Cr | Serum creatinine |
| CsA | Cyclosporine A |
| DBil | Direct bilirubin |
| DED | Dry eye |
| eCRF | Electronic Medical Record Report Form |
| EDC | Electronic Data Capture |
| EDS | Eye dryness score |
| FAS | Full analysis set |
| FDA | Food and Drug Administration (FDA) |
| GCP | Good Clinical Practice (GCP) |
| GLU | Fasting blood glucose |
| Hb | Hemoglobin |
| ICSS | Corneal fluorescein staining score |
| ICF | Informed Consent Form |
| NMPA | National Medical Products Administration |
| PLT | Platelet |
| PPS | Per Protocol Set |
| RBC | Erythrocyte |
| SAE | Serious adverse event |
| SAP | Statistical Analysis Plan |
| Schirmer | Schirmer I Test |
| SOP | Standard Operating Procedure |
| SS | Safety Set |
| SUSAR | Suspicious and Unexpected Serious Adverse |
| | Reactions |
| TBil | Total bilirubin |
| UA | Uric acid |
| UREA | Urea |
| VAS | Visual Analogue Scale |
| WBC | Leukocyte |

### Example 8 Analysis of Early Phase Efficacy

In this example, the early phase efficacy of multiple CsA drugs were analyzed and compared. Specifically, the CsA drugs analyzed were OTX-101 (CEQUA™, 0.09% nanomicellar CsA solution), RESTASIS (0.05% CsA emulsion), Ikervis (0.1% CsA cationic emulsion), CyclASol (0.1% CsA, waterfree cyclosporine formulation), and the 0.05% CsA Gel formulation. Data was collected from the Phase II and/or Phase III clinical trial studies of these drugs. (Sheppard, J., et al. “Phase 3 efficacy (worse-eye analysis) and long-term safety evaluation of OTX-101 in patients with keratoconjunctivitis sicca,” Clinical Ophthalmology (Auckland, NZ) 15: 129 (2021); Goldberg, Damien F., et al. “A Phase 3, randomized, double-masked study of OTX-101 ophthalmic solution 0.09% in the treatment of dry eye disease,” Ophthalmology 126.9: 1230-1237 (2019); Stevenson, D, et al. “Efficacy and safety of cyclosporin A ophthalmic emulsion in the treatment of moderate-to-severe dry eye disease: a dose-ranging, randomized trial.” Ophthalmology, 107.5: 967-974 (2000); Sall, K., et al. “Two multicenter, randomized studies of the efficacy and safety of cyclosporine ophthalmic emulsion in moderate to severe dry eye disease,” Ophthalmology, 107.4: 631-639 (2000); Baudouin, C., et al. “A randomized study of the efficacy and safety of 0.1% cyclosporine A cationic emulsion in treatment of moderate to severe dry eye,” European Journal of Ophthalmology, 27.5: 520-530 (2017); Leonardi, A., et al. “Efficacy and safety of 0.1% cyclosporine A cationic emulsion in the treatment of severe dry eye disease: a multicenter randomized trial.” European Journal of Ophthalmology, 26.4: 287-296 (2016); Wirta, David L., et al. “A clinical phase II study to assess efficacy, safety, and tolerability of waterfree cyclosporine formulation for treatment of dry eye disease,” Ophthalmology, 126.6: 792-800 (2019); Sheppard, John D., et al. “A Water-free 0.1% Cyclosporine A Solution for Treatment of Dry Eye Disease: Results of the Randomized Phase 2B/3 ESSENCE Study,” Cornea 40.10: 1290 (2021))

P-values were presented to show whether the change of a certain clinical parameter in the test group is statistically significant compared to that in the vehicle group. P<0.05 indicates significance. In the present context, p<0.05 means that the tested CsA drug shows efficacy in terms of that clinical parameter.

Most CsA drugs showed no significant efficacy in terms of most clinical parameters compared with the vehicle in the earliest visit (Week 2, Week 4 or Month 1). (Tables 48-55) However, the 0.05% CsA gel shows early efficacy as early as week 2 (D14) in 5 out of 6 clinical parameters tested. (Table 56) This result suggests that a method of treating moderate to severe dry eye disease with CsA gel can achieve early onset of efficacy. In some embodiments, the onset of efficacy starts no later than D14. In some embodiments, the onset of efficacy starts no later than D30.

TABLE 48
OTX-101 (CEQUA ™, 0.09% nanomicellar CsA solution) in Phase II

| | Cequa vs. | Cequa vs. | Cequa vs. | Cequa vs. | Cequa vs. |
|---|---|---|---|---|---|
| | Vehicle | Vehicle | Vehicle | Vehicle | Vehicle |
| | Week 2 | Week 4 | Week 6 | Week 8 | Week 12 |
| lissamine green | P > 0.05 | P > 0.05 | P = 0.048 | P = 0.029 | P = 0.0076 |
| conjunctival staining | | | | | |
| sum score | | | | | |
| corneal fluorescein | P > 0.05 | P = 0.015 | P > 0.05 | P > 0.05 | P = 0.003 |
| staining sum score | | | | | |
| BUT | P > 0.05 | P > 0.05 | P > 0.05 | P > 0.05 | P > 0.05 |
| Schirmer's test | N/A | N/A | N/A | N/A | P = 0.0003 |
| Number (%) of | N/A | N/A | N/A | N/A | 17.9% V.S 7.6%, |
| patients with an | | | | | P = 0.007 |
| increase in Schirmer's | | | | | |
| scores of ≥10 mm | | | | | |
| Global symptom score | P > 0.05 | P > 0.05 | P > 0.05 | P > 0.05 | P > 0.05 |

TABLE 49
OTX-101 (CEQUA ™, 0.09% nanomicellar CsA solution) in Phase III

| | Cequa vs. | Cequa vs. | Cequa vs. |
|---|---|---|---|
| | Vehicle | Vehicle | Vehicle |
| | Week 4 | Week 8 | Week 12 |
| lissamine green | P > 0.05 | P < 0.001 | P = 0.007 |
| conjunctival staining sum | | | |
| score | | | |
| corneal fluorescein | P < 0.01 | P < 0.01 | P < 0.01 |
| staining sum score | | | |
| Number (%) of patient | P = 0.04 | P = 0.003 | P = 0.02 |
| with complete clearing of | | | |
| central corneal staining | | | |
| Number (%) of patients | N/A | N/A | 16.6% vs. |
| with an increase in | | | 79.2%, |
| Schirmer's scores of ≥10 | | | P < 0.0001 |
| mm | | | |
| Global symptom score | P > 0.05 | P > 0.05 | P > 0.05 |

TABLE 50
RESTASIS (0.05% CsA emulsion) in Phase II

| | RESTASIS vs. | RESTASIS vs. | RESTASIS vs. |
|---|---|---|---|
| | Vehicle | Vehicle | Vehicle |
| | Week 4 | Week 8 | Week 12 |
| temporal rose Bengal | P > 0.05 | P > 0.05 | P > 0.05 |
| staining | | | |
| corneal fluorescein | P > 0.05 | P > 0.05 | P > 0.05 |
| staining sum score | | | |
| Schirmer's test | P > 0.05 | P > 0.05 | P > 0.05 |
| BUT | P > 0.05 | P > 0.05 | P > 0.05 |
| ocular dryness score | P > 0.05 | P > 0.05 | P > 0.05 |
| OSDI score | P > 0.05 | P > 0.05 | P > 0.05 |

TABLE 51
RESTASIS (0.05% CsA emulsion) in Phase III

| | RESTASIS vs. | RESTASIS vs. | RESTASIS vs. | RESTASIS vs. |
|---|---|---|---|---|
| | Vehicle | Vehicle | Vehicle | Vehicle |
| | Month 1 | Month 2 | Month 4 | Month 6 |
| lissamine green | P > 0.05 | P > 0.05 | P > 0.05 | P > 0.05 |
| conjunctival staining sum | | | | |
| score | | | | |
| corneal fluorescein | P > 0.05 | P > 0.05 | P ≤ 0.05 | P ≤ 0.05 |
| staining sum score | | | | |
| Schirmer's test | N/A | N/A | P < 0.05 | P < 0.05 |
| OSDI score | P > 0.05 | P > 0.05 | P > 0.05 | P > 0.05 |

TABLE 52
Ikervis (0.1% CsA cationic emulsion) in Phase III by SCCICANOVE

| | Ikervis | Ikervis | Ikervis |
|---|---|---|---|
| | vs. | vs. | vs. |
| | Vehicle | Vehicle | Vehicle |
| | Month 1 | Month 3 | Month 6 |
| lissamine green | P > 0.05 | P > 0.05 | P > 0.05 |
| conjunctival | | | |
| staining sum score | | | |
| corneal fluorescein | P = 0.002 | P = 0.03 | P = 0.009 |
| staining sum score | | | |
| OSDI score | P > 0.05 | P > 0.05 | P > 0.05 |

TABLE 53
Ikervis (0.1% CsA cationic emulsion) in Phase III by SANSICA

| | Ikervis vs. Vehicle | Ikervis vs. Vehicle | Ikervis vs. Vehicle |
|---|---|---|---|
| | Month 1 | Month 3 | Month 6 |
| conjunctival cell surface impression | P = 0.019 | N/A | P = 0.021 |
| cytology | | | |
| corneal fluorescein staining | P > 0.05 | P = 0.024 | P = 0.037 |
| sum score | | | |
| tear film osmolarity | P > 0.05 | IN/A | P = 0.048 |

TABLE 54
CyclASol (0.1% CsA, waterfree cyclosporine formulation) in Phase II

| | CyclASol vs. | CyclASol vs. | CyclASol vs. | CyclASol vs. |
|---|---|---|---|---|
| | Vehicle | Vehicle | Vehicle | Vehicle |
| | Week 2 | Week 4 | Week 12 | Week 16 |
| corneal fluorescein staining sum | P > 0.05 | P > 0.05 | P > 0.05 | P > 0.05 |
| score | | | | |
| central corneal staining | P > 0.05 | P < 0.05 | P > 0.05 | P > 0.05 |
| score | | | | |
| lissamine green conjunctival | P > 0.05 | P > 0.05 | P > 0.05 | P > 0.05 |
| staining sum score | | | | |
| OSDI score | P > 0.05 | P > 0.05 | P> 0.05 | P > 0.05 |

TABLE 55
CyclASol (0.1% CsA, waterfree cyclosporine formulation) in Phase III

| | CyclASol vs. | CyclASol vs. | | |
|---|---|---|---|---|
| | Vehicle | Vehicle | CyclASol vs. Vehicle | CyclASol vs. Vehicle |
| | Week 2 | Week 4 | Week 12 | Week 16 |
| corneal fluorescein | P = 0.043 | P = 0.0002 | P = 0.002 | P = 0.011 |
| staining sum score | | | | |
| lissamine green | P > 0.05 | P < 0.05 | N/A | N/A |
| conjunctival staining | | | | |
| sum score | | | | |
| Number (%) of patients | P > 0.05 | P > 0.05 | P > 0.05 | P > 0.05 |
| with an increase in | | | | |
| Schirmer's scores of | | | | |
| ≥10 mm | | | | |
| OSDI scrore | P > 0.05 | P > 0.05 | P > 0.05 | P > 0.05 |

TABLE 56
0.05% CsA Gel Formulation in Phase III (Example 7)

| | CsA vs. Vehicle | CsA vs. Vehicle | CsA vs. Vehicle |
|---|---|---|---|
| | Week 2 | Week 6 | Week 12 |
| proportion of patient with ICSS decrease | P = 0.0052 | P = 0.0001 | P = 0.0001 |
| from baseline ≥ 1 point | | | |
| ICSS change from baseline | P = 0.002 | P = 0.0001 | P = 0.0001 |
| corneal fluorescein staining sum score | P = 0.0186 | P = 0.0072 | P = 0.0029 |
| (Oxford grading) | | | |
| BUT | P = 0.0702 | P = 0.0012 | P = 0.0002 |
| Schirmer's test | P = 0.0000 | P = 0.0003 | P = 0.0002 |
| Number (%) of patients with an increase | 9.9% vs. 5.2%, | 11.5% vs. 9.9%, | 15.1% vs. |
| in Schirmer's scores of ≥10 mm | P = 0.025 | P > 0.05 | 10.3%, P < 0.05 |

1. A method of treating moderate to severe dry eye disease in a subject in need thereof, comprising administering to the subject an effective amount of cyclosporine A ophthalmic gel.

2. The method of claim 1, wherein the subject is characterized in a) Eye dryness score (EDS) ≥40 points (visual analogue scale [VAS] of 0-100 points); b) Break-up time (BUT) <10 s; c) Schirmer test result <10 mm/5 min; and d) Corneal fluorescein staining score (ICSS) ≥2 points.

3. The method of claim 1, wherein the subject does not have an alanine aminotransferase (ALT) level ≥2 times an upper limit of normal, an aspartate aminotransferase (AST) level ≥2 times an upper limit of normal, or a serum creatinine (Cr) level ≥1.5 times an upper limit of normal.

4. The method of claim 1, wherein the cyclosporine A ophthalmic gel is administered to the subject once a day.

5. The method of claim 1, wherein an effective amount of the cyclosporine A ophthalmic gel is one drop per day.

6. The method of claim 1, wherein a concentration of cyclosporine A in the cyclosporine A ophthalmic gel ranges from about 0.05% to about 0.1% by weight.

7. The method of claim 1, wherein a concentration of cyclosporine A in the cyclosporine A ophthalmic gel is about 0.05% by weight.

8. The method of claim 1, wherein an effective amount of cyclosporine A ophthalmic gel administered to the subject per day contains about 0.025 mg cyclosporine A.

9. The method of claim 1, wherein the cyclosporine A ophthalmic gel is administered every night before the subject goes to bed.

10. The method of claim 1, wherein a time window between administration of the cyclosporine A ophthalmic gel and administration of a different drug is no less than 5 minutes.

11. The method of claim 2, wherein at day 84 since the administrating starts, the subject's ICSS drops by at least 1 point from the subject's ICSS before said administering.

12. The method of claim 1, wherein a treatment period of the method is up to about 12 weeks.

13. The method of claim 1, wherein in a treatment period, onset of efficacy starts no more than 30 days since first administration of the cyclosporine A ophthalmic gel to the subject.

14. The method of claim 1, wherein in a treatment period, onset of efficacy starts no more than 14 days since first administration of the cyclosporine A ophthalmic gel to the subject.
